SEQUENCE LISTING (30 sequences)

SEQ ID NO: 1 - DNA, 1368 bp, Helichrysum umbraculigerum
  source 1..1368: mol_type = other DNA
atggcaacccaagtcaaaaccgaggagaagcatttgaaggtagaaatcataaacaaaacctatgtgaaacctgaaacaccactaggaagaaaagagtgtcaattggtcacatttgatcttccttatatagccttctactacaaccaaaagttgatcatctataaaggtggtgtcgaggagttcgaggataccgtcgagaaactgaaagacgggttaaaggtagttttgggagagtttcatcaattggctggaaaattagacaaagatgatgacggggtgtttaaggtagtgtacgatgatgacatggatggggtggaggtgctttctgcggtcgcggaagacactgcgaccgcagatttgatggacgaagaagggaccatcaagcttaaggagttggtcccttataatagtgttttgaacatagaggggcttcatcgtccgcttttatcgattcagataacaaaactaaaagatgggcttgtactgggctgtgcgttcaaccacgcgattttagacggtacatccacctggcacttcatgagctcctgggcccaaatttgctccggatccaaatccatttcagcggcgcctttccttgaccgtacccaagcgcgtaacacgcgcgtgaaactcgatctcacccctcccgcccaaactaacggcaattcaaacggcgacactaacggtgatgcgagcgccacgaagccaccagcaccggcaccgttaagagaaaaaatcttcaaattctcagagtcagcaatcgacaaaatcaaagcaaaaatcaatgcgaatccaccggaaggatcaaccaagccattctccacatttcaatcgctctccacacacatatggcacgcagttacacgcgctcgcaatctaaaaccggaagactacaccgttttcactgttttcgccgattgccggaaacgtgtcgatcctccgatgccggatagctatttcggaaacctaattcaagcgatcttcaccgtcaccgctgccggattattgcaggcgaatccaccggaattcgcggcgtcaatgatacaaaaagcgattgatatgcacgatgcgaaagcaattgaagcgcgtaacaaagaatgggaaagtaatccgattatatttcaatacaaagacgccggagttaattgtgttgcggttgggagttcacctaggtttaaggtttatgatgtggatttcgggtttggtaaacccgaaagtgttcggagcggggcgaataaccggtttgatggtatggtttatttgtatcagggaaaaagtggtggaaggagtattgatgtggagattagtttggatgcaagtgcaatgggaaatcttgaaaaggataaggaatttcttatccaagaataa

SEQ ID NO: 2 - DNA, 1353 bp, Helichrysum umbraculigerum
  source 1..1353: mol_type = other DNA
atggcttctcttcctctcttaactgttcttgaacaatcccatgtatcaccaccgccagccaccgtagtcgataaatcgttgtcgctaacctttttcgatttcctgtggctaactcaacctccaattcacaatcttttcttttacgagttttcaatcgacgaaactcagttcgtggaaactatcgttcctagtcttaaaaactcgttatcaatcactcttcaacatttttacccgttcgccggtaaccttatcttatttcctgataacaaaaggcctgaaattcgttacgttgaaggtgattatgtcatggttacatttgcaaaatctagccttgacttcaatgaactagtaggaaaccatcctagagattgtgaccagttttatgatcttattcctccattaggtgaaagtgtgaaaacttctgaatttcgaaaaatcccactcttttcggtccaggtgacgttttttccacaaaaaggcgtatcgattggtatgacgaatcatcatagtcttggcgatgctagcactcggttttgtttcttgaacgcgtggacatcgatttctagatctagttcagatgagtcatttctagcaaacggaactaaaccgttttacgatagagtgataagtaacccgaaactagatcaaagttatctaaaattttccaagatcgatactctttacgagaagtatcaacctttaagcctctctagaccatctaataaacttcgtggcacgtttatcttgacgcgaaaaatcctaaacgagttgaaaaaaagtgtgtcaattaaactaccaactttatcatatgtatcatcttttacggttgcatgtggttatatttggagttgcatagcgaaatcacgaaacgatgatctacaactattcgggttcactattgattgtagggcacgtttggatccaccggttccatcaacttattttgggaattgtgtcgggggttgtatggcgatggcaaaaacaacgttgttaaccgaagacgatggatttataacggctgctaaattgcttggagaaagtttacacaagacgttgaccgaatcgggtggaatcgtgaaagatatagaagtgtttgaagatttgtttaaggatggattaccaacaactatgataggagttgcgggaacaccaaagcttaagttttatgagacggatttcgggtgggggaacccgaaaaaggtggaaacgatttcgattgattataacatgtcgatttctatgaacgcttgtagagaatcgaaggatgatttggagattggtgtttgccttatgaatactgaaatggaagcttttgttcgtttatttgatgaaggattagaatcatacgtttag

SEQ ID NO: 3 - DNA, 1329 bp, Helichrysum umbraculigerum
  source 1..1329: mol_type = other DNA
atgggaagtgaaaatgttcacaaaataatgaaaatcaacatcactaaatcatcatttgtacaaccctcaaagcctacagtactacccactaaccacatatggacttctaacttagatttagttgtgggtagaattcatattttaaccgtttacttttaccgtccaaatggtgcttcgaatttttttgatccaattgttatgaaaaaagctttagctgatgtgcttgtttctttttatccgatggccggaagaataagtaaagatgataatggtagagttgtaattaattgtaatgatgaaggtgttttgtttgttgaagctgagtcagattccacgttggatgacttcggtgagtttacaccgtctccggagctccgacaacttaccccgacgattgattactccggtgacatttcaacgtacccgctattttttgcacaggtaacgcatttcaagtgtggaggagttggttttggttgtggtgtgtttcatacacttgcagatggtctatcctctatacatttcatcaacacatggtcggacatggctcgtggtctctcgatagccatcccgccattcactgaccggacccttcttcgtgcacgtgaaccacccactcccacttttgaccacgtagagtaccacctccctccgtccatgaaaactacctcacaaaccaacaaatccagaaagccttccacggccatgttaaagcttacgcttgatcaactaaatgctctcaaagctgctgctaagaatgaaggcggcaacaccaattatagcacgtacgagatcctggcggctcatttatggcggtgtgcctgcaaggctcgaggactccctgatgaccaactaaccaaattgtacgtggcaacagatggacggtccagattgagccctcaactcccaccaggctatctaggcaatgttgtgttcaccgccaccccagttgccaaatcagctgacctcacgactcaaccattgtctaatgcagcatctttgatccgaaccacattgacaaaaatggataacgactatttgagatctgccattgattaccttgaggtgcagccagatctatctgctttaattcgtggtcctagttactttgctagcccgaatttgaacataaacacgtggacccggttgccagtacatgatgcggatttcgggtggggtcggcctgttttcatgggaccagcagtgatattgtatgagggcaccatctatgttctaccaagcccaaacaatgataggagtatgtcattggcagtctgtttagatgcagatgaacaaccatcgtttgagaagttcctgtatgacttttaa

SEQ ID NO: 4 - DNA, 1425 bp, Helichrysum umbraculigerum
  source 1..1425: mol_type = other DNA
atgccttcatcatcatcatcgccttcttcaacagctgattcagttaccataatctcaaaatgcacagtctacccacatatgaaaaactcaacaccagaatccttgcagctctctgtttctgatctcccaatgctttcatgtcaatacatacaaaaaggtgtcttactttctcaaccgccacccaatcacaccaacaatatcatttcccacttaaaactctctctctctaaaaccctctctcacttcccacctctcgccggccgtctttcgaccgactctcacggccacgtctctatcatctgcaacgattccggcgtcgaattcgttcactccaccgctaaccacctccacacccaccaaatcttacccctcaattccgacgttcacccatgttttaaaaccttttttgcttttgataaaactctgagttacgccggccaccaccaaccaatcgccgccgtgcaagtcacggagcttgctgatggactctttattgggtgtacggtaaatcatgctgtcgttgacgggacttctttttggaacttttttaatacttttgctgagatcacaaaagggtgtcagaaagtaacgaacttgccggattttagccgggaaaatgttttcatttctccggttgttttgcctcttccctccggcggcccgtcggcgacgttctcaggtgatgagccgttgagggaaaggatcattcatttcagtagagacgcgattctgaagatgaaattcagagctaataatcctttatggcggcaaccacaaaattcggatctggatgatacagagatttacgggaaagtgtgtaacgacattaacggcaaagttaacggggcgtttaaacccaaaagtgaaatttcgtccttccagtctttatgtggtcagttatggcgtgcggttacacgcgcgcgtaaattcaacgaccctataaaaacgacgacgtttcgaatggcggtgaattgtaggcataggctagacccaaaggtcgacaaactttatttcgggaacttgatccaaagcatcccgaccgttgcttcagttggggagttgttatcacatgatttgtcgtgggcagccaatgagcttcaccaaaatgtggtggcgcatgataatgctaccgtgcgcaggggtgttaaggattgggagaataatccaaagttgtttcctttggggaattttgatggtgctatgatcacaatgggaagttctcctaggtttccaatgtataataacgatttcgggtggggccgcccaatggcggttcgtagtggtaaagctaataagtttgatggaaagatttcggcttttccgggacgtgatggtgatggtagtgtcgatcttgaggttgttttagctcccgaaaccatggcatgtcttgaacgtgaccatgaatttatgcaatatgtatcttaa

SEQ ID NO: 5 - DNA, 1608 bp, Helichrysum umbraculigerum
  source 1..1608: mol_type = other DNA
atgaagtggtttttcataacccataaagcaacccagcgttgccttaattctaaacaatttcatcttcacggaggttcgaatttcgtttccggtaatagatgttttcttgcatcacactcaatggagcggccaaaattcatgttgataccatattatccctaccaaattcggtccttaaattcgagtcaccgatatagtagtacgtcacccagcggatcccctcacagttttctgaatggtactaagaatgaaaactatacgaagaaggtagatcttgaaataatttcaagagaaatcatcaaaccagcttctccaactccacatcatttaagaaacttcaacttatcacttttggaccaaatagtatttgattgctacacccctgtaatcctctttattccaaatagtaataaggctactgttacggatgtcatgatcaaaagattgaaacatctcaaggagactttatctcgaattctaagtcaattttatccctttgcgggagaagttaaggacagattgcatatcgaatgcaatgacaagggagtcaattacatcgaggctcaaatcaatgagacattggaagaatttctatgtcatccagataacgaaaaggcgagggagcttatgcccgaaagccctcatgttcaagaatctgcaataggaaactatgctatgggtattcagataaacattttcagttgcggagggattggactttccatgagcatggcacacaagatcatggacttctacacatatacgatcttcatgaaagcatgggctgcagctgttcgaggttcaccagatacaattatttcaccaagttttgtggcttctgaggtctttcctaatgatcccagccaagaagattcaattcctatcgagttaaagtctagtaatttgcttagcacaaaaagatttgagtttgatcctactgcgttggctctcctaaagggacaagttgtcgccagcggatcacctccccaacgaggaccaagtcgtatggaggcgacaacagccgttatttggaaggccgctgcaaaagctgcatcgactgtcagaagattcgatccaaagtcacctcatgcgctggcgttaccagtaaatatacgtaaaagggcatcacctgctctcccagacaattccataggaaacatagttatgcgaggtatagcaatttgttttcctgagagccaaccggacttgccaactcttatgggtaaagtgagagaatcaatagcgaaacttaactcagattacattgagtccctgaaaggtgaaaaggggcatgagacagttaataagatgttgaaggagttgaagcttcggacgaatatgacaaaggtaggagggaaattcgttgctagttgcatatttaatagtggaatatatgagttggatttcgggtggggaaaaccgatatggttctatgttgtgaatccaggaagcgatagttgtgtggttttgactgatacgctgaagggtggtggtgttgaagccacaattacactaccaccagatgaaatggagatattcgaacgtgatcatgagcttctatcctatactaccatcaaccctagtccactgcgatttcttgaccattga

SEQ ID NO: 6 - DNA, 1395 bp, Helichrysum umbraculigerum
  source 1..1395: mol_type = other DNA
atggaggtgcctgaccaattccacctaaacattcttgaacaatgccacgtttcaccatcaccaaattccatcataccttcattttcactacccttaacattcttagacatcccatggcttttttacccttcaaatcaaaccctttttttcttcccagaaccaccacccaaaaccaccatcatcaccacccttaaacaatcactctctcttaccctccaccacttccaccctctcgccggaaacctctcacttccatcacctccggcggaaccccacattgtttacaccaaaaatgactcaattgcactcacaattgctcaaacaaacaccaacatccaccatctttcttgcaatcacccaagaagtgtaaaaaatctttactctcttttacccaaactcccatctccatccatgtcacgtgaaactcacgtgggccttgttatcccccttcttaccatccaaattacggtttttgctgatttggggtattcgatcggagtcactatgcaacatgcagcagttgatgaacggacatttgatcagtttatgaaatgttgggcgtctgtttgtacatctttgttgaaaaatgactcactttttacattcaagtctacaccttggtacgataggagcgtaattatcgaccccaaatcgctgaaaacaacgtttttaaagcaatggtggaaccgatctaattctctcaatgagtcacatgatcaagaaaatgatgatcatgatcttgttctagcaacttttgttttgagttcattagatattaacatgatcaagaatcatattcttgcaaaatgcaagatgataaatgaggatccaccactacatttatctccttatgttagtgcatgtgcttatttatggaaatgtttaatcaaaattcaagaaacccatgattctattaagggtggtcctctctatttagggtttaatgccggtgggattactcgattagggtacgacataccttcaacttattttgggaattgtatagcttttgggagatgcaaggcatttgagagtgaattattgggtgataatggtattgttttcgcggcaaaatcgattggaaaagagatcaagaggcttgataaggatgttttaggaggtgctaataagtggattagtgattgggatgaattaaccattaggcttcttggttcaccaaaagttgattcatatggtatggattttggatggggtaaagttgagaaggttgaaaaaatatcaagtatttcaaatcacggtagggttaatgtaatttctttgagtggatgtaaggattttaaaggtggaatagagataggggttgttctttctgtggctaaaatgaatgttttcacttccctctttcatggaggtttaatggagtttgcatattga

SEQ ID NO: 7 - DNA, 1218 bp, Helichrysum umbraculigerum
  source 1..1218: mol_type = other DNA
atgaaaaataagaacccgactagtgtgatcagagaggctttagctaaggtattggtgttttattatccgtttgctggccggctcaaggaagggccggccaggaaactgatggtggattgttctggtgaaggtgtgttgtttattgaggcagaagctgatgtcacgttgaaacaatttggtgacgcacttcaaccgccatttccttgtttagaagagcttctttacgatgttcctggatctactggtattctagatacaccattattgctgattcaggtgacacgattgttatgtggaggttttatctttgctctacgactcaaccacaccatgagcgacgcagcaggtctcgttcaattcatgacagggcttggtgaaatggcacaaggtgcatcaaggccatcaacgttgcctgtatggcaaagggagttgctttttgcaagggacccaccacgcgtgacttgtactcatcacgagtatactgaagtggaagacaccaatggtacaatcattccgctagatgacatggcacataaatcatttttctttggaccttctgagatatcagcgttgcgaaggttcgttccatcatacctaaaaaagtgttctacttttgaggtcttaaccgcttgcctatggcgttgtcgtacaattgcactccagccagatcccgaagaagagatgcgcatgatatgcattgttaatgcgcgtggaaagtttaatcctcccctattacccaaaggatattatggaaatggtttcgctataccagtggccatttcaacagctggagacctatctagcaaaccattaggtcacgcattggaacttgtaatgaaagccaaatccaatgtcactgaggagtatatgagatcagtagccgacttaatggtaatcaagggacgaccccactatacggttgtccgaagctaccttgtatcggatgtgactcacgctggatttgatgttgttgatttcgggtgggggaaagcgtcctatggaggacctgcaaaagggggagtaggtgctattcccggagttgttactttctttataccttttacaaaccataaaggcgagtctggaattgtgctacctatatgtttgccgagtgcagccatggataagtttgttgaagagttaaataagatgttggtcccagacaacaacgaacaagtactccgagaacacaagttactagttctcgctagattgtaa

SEQ ID NO: 8 - DNA, 1386 bp, Helichrysum umbraculigerum
  source 1..1386: mol_type = other DNA
atggcacaaatcgacactccattgacattcaaagtccggagacatgcaccggagctgatcgctccagcgaaacctacgccacgagaactaaaacctctatccgacattgatgatcaagaaggccttaggtttcatatcccagtgattcaattctatcgtagcgatccaaagatgaaaaataagaacccggctagtgtgatcagagaggctttagctaaggtgttggtgttttactatccgtttgctggccggctcaaggaagggcctgccaggaaactgatggtagattgctctggtgaaggtgtgttgtttattgaggcggaagctgatgtcacgttgaaacaatttggtgacgcccttcaaccgccgtttccttgtttggaagagcttctttacgatgttcctggatctactggcgttctagatacaccgttattgctgattcaggtgacacgattgttatgtggaggttttatctttgctctacgactcaatcacaccatgagcgacgcaccaggtctcgttcaattcatgacagggctcggtgaaatggcacaaggtgcatcaaggccatctacgttgcctgtatggcaaagggagttgcttttagcaagggacccaccacgcgtgacatgtactcatcacgagtatactgaagtggaagacaccaagggtacaatcattccgctagatgacatggcacataaatcatttttctttggaccttctgagatatcagcattgcgaaggttcgttccatcatacctaaaaaagtgttctacttttgaggtcttaaccgcttgcctatggcgttgtcgtacaattgcactccagccagatcccgaagaagagatgcgcataatatgcattgttaatgcgcgcggaaagtttaatccaccccttcctaaaggttattatggaaatggttttgctttcccagtggccatttcaacagctggagatctatccagcaaaccattaggtcatgcattggaacttgtaatgaaagccaaatccgatgtcactgaggagtatatgagatcaatagccgacttaatggtaatcaagggacgtccccactttacggttgtcagaagctaccttgtctcggatgtgactcacgctggatttgatgttgttgatttcgggtgggggaaagcggcctatggaggacccgctaaagggggagtaggtgctatcccaggtgttgctagtttctatataccttttacaaaccataaaggcgagtctggaattgtgctacctatatgtttgccgagtgcggccatggataagtttgttgaagagttaaataagatgttggtcccagacaacaacgaacaagtactccgagaacacaagttactagttcttgctagattgtaa

SEQ ID NO: 9 - DNA, 1332 bp, Helichrysum umbraculigerum
  source 1..1332: mol_type = other DNA
atggaaatacaagtaataaactactcatcaaagctagtaaaacccttgacaccaacacccaccgcaaatcgttactataacatttctttcaccgatgagctcgtcccaaccatttacgtcccactcattctctactacgcaacaccgaaaaacccaaatggtgatcactttgaaaacatttgtgaccgtctggaggagtcgttatcgaaaacgttaagtgatttttacccactggccgcgagattcattcgtaaactctccttaattgattgtaacgatcaaggggttttgtttgtcctaggcaatgtaaatatccgactttcggatgttacaggcctaggactgacgtttaaaaccagtgttttaaatgattttctcccgtgtgagattggaggagcggatgaagtcgatgatcctatgctttgtgtcaaagtcaccacttttgagtgtggtggttttgcaattggtatgtgtttttcgcataggctttcggatatgggtaccatgtgtaactttattaacaattgggctgctagaactattggtgaatatgataatgaaaaacatactcctatttttaattcgccgttgtacttcccgcaacgaggattacctgaacttgacctaaaagtacctaggtcaagtattggtgtgaaaaatgcagcacgcatgtttcactttaatgggaaggcaatatcatccatgagagaagtttttggagttgatgaaaatgggtctcgtagactctcaaaggttcaacttgttgtagccttgttgtggaaggcctttgttcgcatagatgatgtgaacgatggccaatctaaggcgtcttttctgatccaaccagttgggttgagggacaaagttgtccctccattaccatcaaactcatttgggaatttttggggtctagcgacttcccaacttggtcctggtgagggtcacaaaatcggtttccaagaatatttttacattttgcgtgaatctattaagaaaagagctagggattgcgctaaaatattgacacacggtgaagaaggatatggggttgtaatcgatccatatcttgagtcgaatcaaaagatagctgataatggtacaaacttttacttgttcacttgttggtgcaagttttcgttctacgaagctgattttggttgtggtaagccgatttgggctagcaccggaaagtttccggttcaaaatttggtgatcatgatggatgataatgagggtgatggtgtagaagcgtgggttcatttagacgataaacgcatgaatgagttagaacaagatcctgatgttaaactctacgcatgcaatttagcttaa

SEQ ID NO: 10 - DNA, 1305 bp, Helichrysum umbraculigerum
  source 1..1305: mol_type = other DNA
atgaaattagcagtgaaggaatcagtgatagtaaaaccatccaaaacgacaccgtgtcagcaaatatggacatcaaatcttgatttagtggtgggtcggatccatatattaaccgtttacctttacagaccaaatgggtcttcaaatttctttgattccatggttttaaagaaggctctagccgacgttttagtttctttttttccggtggccggacggttggataaagacggtgacggcagagttgtaatagattgtaacggtgagggtgttttgtttgtggaagctgaagctgattgttgcattgatgattttggtgagattactccgtcgccggagttacgacggttggtgccgacggtggattattccggtgatatgtcttcttatccgttatttattacgcaggttacacggttcaagtgtgggggagtttcgttaggctgtggactacaccatacgttatcggatggactctcagcacttcacttcatcaacacatggtctgatgtagctagaggcctatcggtggcaatcccaccgttcattgaccgctcccttcttcgagctcgtgatccaccatcccctgtgtttgaccacatcgaataccacccaccaccgtcactgatcactccgttgcaaaaccaaaagaacgcgtcacattcgaggtctgcttcaactttaatcctacggctcacactccatcaaataaacaatcttaaatcaaaggctaaaggcgatgggagcatgtaccatagcacgtacgagatcctagctgctcatctatggcgatgtgcgtgcaaagcacgtggactagcaaacgatcaaccaaccaaattgtatgtggccaccgatggacggtcaagattgattcctccactccctccgggctaccttgggaatgtcgttttcacggctactcctgtcgctaaatcgggagatttcgaatctgaatccttggcagagacagcaaggaggattcgcagtgagttgggtaaaatgaacgatgagtatcttagatcagctattgactacttagagtcggtatctgatatttcgacccttgttagagggccgacttactttgcgagtccaaatctgaatgtaaacagttggactcggttaccaatatacgaatctgacttcggttggggtcgacctattttcatgggacccgcaagtatactttacgagggtacgatttacatcataccgagccctagtggtgatcggagtgtgtctctggccgtgtgcttggaccctgatcacatggctttgtttaaagaatgcttgtacgttttttag

SEQ ID NO: 11 - DNA, 1305 bp, Helichrysum umbraculigerum
  source 1..1305: mol_type = other DNA
atgaagctagcagtgaaggaatcagtgatagtaaaaccatccaaaacgacaccgtgtcagcaaatacggacatcaaatcttgatttagtggcgggtcggatccatatattagtcgttttcttttacagaccaaatgggtcttcgaatttctttgattccttggttttaaagaaggctctcgccgacgttttagttcctttttttccggtggccggacggttcagtgaagacggtgacggcagagttgtaattgattgtaacggtgagggtgttttgtttgtggaatctgaagctgattgttgcattgatgattttggtgagattactctgtcgccggagttacaacagttggtgccgacggtggattattccggtgatatgtcttcttatccgttatttattgcgcaggtcacacggttcaagtgtgggggagtttcgttaggttggggactacaccatacattattggatggactctcagcacttcacttcgtcaacacatggggtgatgtagctagaggcctatcggtggcaatccaaccgttcattgaccgctcccttcttcgagctcgtgatccaccgacccctgtgtttgaccacatcgaataccacccaccaccgtcactgatcactccattgcaaaaccaaaagaacgcatcacattcgaggtctgcttcaactttaatcctacagctcacacccgatcaaataaagaatcttaaatcaaaggctaaaggcgatgggagcatgtaccatagcacatacgagatcctagctgctcatctatggcgatgtgcgtgcaaagcgcgtggactagcaaacgatcaaccaaccaaattgtatgtggccgccaatggacggtcaagattgattcctccactccctccgggctaccttgggaatgtcgttttcaacgctactcatgtcgctaaatcgggggattttgaatctgaatccttggcagagactgcaaggaggattcactgtgagttgggtaaaatgaacgatgagtattttagatcagctatcgactacttagagtcggtagatgatatttcaacccttgtcaaagggccgacttactttgcgagtccaaatctgaatgtatacagttggattgggataccaatatatgcatgtgacttcggatggggtcaacctattttcatgagacccgcaagtttcctttacgatggttccatttacatcataccgagccctagtggtgatcggagtgtgttgttggccgtgtgcttggaccctgatcacatggatttgtttaaagaatgcttgtacgctttttag

SEQ ID NO: 12 - DNA, 1383 bp, Helichrysum umbraculigerum
  source 1..1383: mol_type = other DNA
atggtgatgattagcaagcttttacgattaggtagaagaaaacttcacacaattgtatcaagagataccattagaccttcttctccaactccctctcattccaaaacatataatctctccttgctcgatcaaatagctgtaaattcatacgtgccgattgttgctttttacccaagctcaaatgtttgtcgaagttccgatgataagacgctggagttgaagaactcattatcgaaaatattaactcattactatccgtttgccggtagaatgaagaagaatcgccctaccgtcgttgattgcaatgatgaaggggttgagttcgttgaagcacgtaataccaactcgttatcagatttcctccaacaatcggagcacgaagatctagatcaactctttccagatgattgtgtatggttcaaacaaaaccttaaaggttctattaatgacgcaaataatagtagcgtatgtccattgagcattcaagtcaaccatttcgcgtgtggaggtgtagcagttgcaacttcgttacgccacaagattggagacggaagcagtgcgttaaatttcattaaacactgggctgcagttacgtcacactctcgagcagggaatcatcaaattgatgcgacatcacccatcattaatccccatttcatttcttacccaactagaacttttaaattgccagataggtcaccatacataccacctagtgatgttgtgtcaaaaagttttgttttccccaacacaaatataaaggacctccaagccaaggtggtaaccatgaccatgggctctagacaacctatcgtgaaccctacccgagctgatgtcgtatcatggcttctacataagtgtgtagtagcagcagctaccaaaaggatatcgggaaattttaaagaaagttgcgtgatctcgccattaaatctgagaaacaagttagaagagccattgcctgaaacaagcataggaaatattttctatctgataacctttccaataagcaataatcatggcgatctcatgcccgatgacttcattagccaactcaggctaggaatacgtaagtttcaaaatatacgaaatttggaaactgcattacgaaccgttgaagagatgatatctgaaacttttatcttgggtacggcagaaagcatggatactagttatgtatattcgagcatccgtgggtttccgatgtatgatattgattttgggtgggggaagcccgtaaaagtaaccgttgggggagcccttaagaacttaagtattctgatggacactcctgatgtcaatggcatcgaagcactagtgtctttggataaacaagacatgaagatacttctaaacgaccctgagttgttggccttttgcttgtaa

SEQ ID NO: 13 - DNA, 1323 bp, Helichrysum umbraculigerum
  source 1..1323: mol_type = other DNA
atgagtactagtgacaaaatgaagataacaataagagaatcatcaatgataaaaccatccaaaccgacgccggatcaacggatatggaactcaaatcttgatttggtagtgggtcggatccatatcttgaccctttacttttttaggccaaatgggtcttcggatttctttgattctgaggttttaaagcaatcacttgccgacgttcttgtttctttttttccgatggccggacgattgggattagacggcgatggcagagttgaaattaattgcaacggtgaaggtgttttgtttgttgaagctgaagcggattgtagtattgatgattttggtgagattactccgtcgccggagctacggcggttggcgccaacagtggattattccggcgatatctcatcttatccactcgttattacccaggtaacacatttcaaatgtggtggagtttctcttgggtgtggactacaccatacattatccgatggactttcatctcttcacttcatcaacacatggtccgatgttacccgaggcttacccgttgcgatcccgccattcgtagatcgtacggttcttcgtgctagggacccgccaaccgtggtctttgatcacgtggaataccacactcctccttccatgacctcaagtttggacaaagacaaacctcaatccgaagatgttcatgtttccacttccatgctacggctcacactcgatcaaataaatgcactaaaagcaaaaggcaaaggtgacggaattgtgtaccatagcacatatgaaatcctagctgctcatttatggcgatgtgcgtgtaaagcacgtgggctcctgaatgatcaaatgactaaattgtatgtagctaccgatggacggtccagattgattcccccactcccaccggggtacttaggcaatgtggtcttcaccgccacaccaattgccaaatccggcgagctccaacaggaaccactagctaccactgcaagaaaaattcatacagagttggccaaaatggatgacaagtacctcaggtcggccctcgactacttagagtcacaacaggacttgtcagcactaattcgagggccagcctattttgcgtgccctaacctcaacatcaatagttggactcgccttccaatatatgatgcggactttgggtggggtcggcccatatttatgggacccgccagcatactttacgagggcacgatttacattattccgagccctagtggtgaccgaagtgtgtcgttggctgtgtgcttagacccctctcatatgcctctcttccaaaagtacttgtatgaactttaa

SEQ ID NO: 14 - DNA, 1326 bp, Helichrysum umbraculigerum
  source 1..1326: mol_type = other DNA
atggtgaatgttgagatcatttctaatgaatacataaaaccatcctccccaacaccaccacatcttaaaatatacaatctttccatcttagatcaactcattcctgccccctatgcacctatcatactatattatccgaatcaagatcacattaacgattttgaggttcacgaacggttgaaactactaaaagattcgttatcgaaaacgctaactcgtttttacccattagccggaaccatcaaaggcgatctttccattgattgtaacgatattggtgcttactttgcagtagctcatgtaaatactcgccttgatgtgttcctgaaccatcctgatcttgacctaataaactgttttcttccacgtgggccttacttgaatggttctagtgaaggaagttgtgtgagtaatgttcaagtgaacatttttgagtgttgtgggattgcaattagtttatgcatttctcacaagattcttgatggtgctgcgttgagtacttttcttaaagcatgggcagggacaagttacgggtcgaaagaagtagtgtatccaaacatgagtgcaccatctttatttcctgctaaagatttgtggcttaaagattcatcaatggtcatgtttgggtctttgtttaagatgggtaagtgtagtactaaaagatttgtttttgattcatcaaaattatccttcctcaaagctaaggcatcgctaaatgggctaaaagacccaacccgcgtagaggtggtgtctgctttactatggaagtgtatcatggctgcatctgaagaaaacactggttcttggaagccatctctgttaagccatgtagttaaccttcgcaaaaggttggtttcaactttatcagaagactcaattgggaacttaatttggttagcaagcgcagaatgtagaaccaacgctcaatcccgattgagtgatcttgttgaaaaggtacgtgatagtgtgtcgaaaatcaatagtgagtttgtgaagaaaatacaaggcgataaagggacaaaagtgatggaagagtctctcaagagtatgaaagattgtgcggattatatcgggtttacgagttggtgtaagatggggttttacgatgtggattttggttggggaaagcctgtatgggtttgtggtagcgtttgtgaaggtagcccggtgttcatgaattttgtcatattaatggacacaaaatatggtgatggaatagaagcatgggtgagcttggatgaacacgaaatgcatatcttaaagcataatcccgagctcttggaatatgcatcaatcgatccaagtcctctgcaaatgaataagtga

SEQ ID NO: 15 - DNA, 1365 bp, Helichrysum umbraculigerum
  source 1..1365: mol_type = other DNA
atgggaactatttatcaatctcccatgatcaaatcttctactcccaaaataattgaagacctcaaagttatcatccatgacacattcacaatcttcccacctcacgaaaccgaaaagcggtccatgttcttatcgaacattgaccaagttcttactttcaacgttgaaacggtccatttttttgcagccaaccctgactttccgccacaagtagtggcggaaaagctcaagttggctctaagtaaggcgctggtgccatatgattttttggcagggaggttgaagttgaaccatgagtcgcaacggtttgagtttgattgtaatggtgctggggctcggttcgtggtgggttcgagtgagtttgagttgggtgagattggtgacttggtgtatccaaaccctgggtttagacaattggttcaaaagagttatgataacttggagttacatgaaaagccactatgcattttacagctgacatccttcaagtgtggaggatttgcacttggtgtagcaacaaatcatgccacttttgatggcttaagtttcaaaacatttcttcaaaatcttggttctttggctgctgatcaaccacttgccgtcgatccctgcaacgatcgccacctattggcagcacgatcaccaccaaaagtccaatttgaccaccctgaactcctcaaaatcccaacaggaacagacatcccaaacccaacagtctttgactgcccagaaagtcaacttgacttcaagattttcaacttgacctcagatgacatagcccacttaaaaacgaaagccaaagatgggcctgggtcaaccaatgcaaaaatcactggattcaatgtggttgcagcccatgtatggcggtgcaaagcgttgtcctcagggtcagaatatgaccccgagagagtgtcaaccgtgttatatgctgttgacattcggtcaagattgaacttaccattatcattagctggcaatgcagttcttagtgcatacgcctcggccaaatgcaaagagattgaagaaggcccgttgtcaagactagtggaaatggtgaccgaaggtactaacagaatgactggtgagtatgcaagatcggtgatcgattggggagaggtgaataaagggtttccaaatggggagtttctgatatcgtcatggtggcgattggggtttgctgacgtggaatatccgtggggtaaacctaggtatagttgtcccgtggtttatcataggaaagatataatattactctttccggatattgttggtgccgataacaacaatgaagtgaatgtgttggtggctttgcctggcaaagaaatggagaaatttgagactttatttcataagtttttggcatga

SEQ ID NO: 16 - protein, 455 aa, Helichrysum umbraculigerum
MATQVKTEEKHLKVEIINKTYVKPETPLGRKECQLVTFDLPYIAFYYNQKLIIYKGGVEEFEDTVEKLKDGLKVVLGEFHQLAGKLDKDDDGVFKVVYDDDMDGVEVLSAVAEDTATADLMDEEGTIKLKELVPYNSVLNIEGLHRPLLSIQITKLKDGLVLGCAFNHAILDGTSTWHFMSSWAQICSGSKSISAAPFLDRTQARNTRVKLDLTPPAQTNGNSNGDTNGDASATKPPAPAPLREKIFKFSESAIDKIKAKINANPPEGSTKPFSTFQSLSTHIWHAVTRARNLKPEDYTVFTVFADCRKRVDPPMPDSYFGNLIQAIFTVTAAGLLQANPPEFAASMIQKAIDMHDAKAIEARNKEWESNPIIFQYKDAGVNCVAVGSSPRFKVYDVDFGFGKPESVRSGANNRFDGMVYLYQGKSGGRSIDVEISLDASAMGNLEKDKEFLIQE

SEQ ID NO: 17 - protein, 450 aa, Helichrysum umbraculigerum
MASLPLLTVLEQSHVSPPPATVVDKSLSLTFFDFLWLTQPPIHNLFFYEFSIDETQFVETIVPSLKNSLSITLQHFYPFAGNLILFPDNKRPEIRYVEGDYVMVTFAKSSLDFNELVGNHPRDCDQFYDLIPPLGESVKTSEFRKIPLFSVQVTFFPQKGVSIGMTNHHSLGDASTRFCFLNAWTSISRSSSDESFLANGTKPFYDRVISNPKLDQSYLKFSKIDTLYEKYQPLSLSRPSNKLRGTFILTRKILNELKKSVSIKLPTLSYVSSFTVACGYIWSCIAKSRNDDLQLFGFTIDCRARLDPPVPSTYFGNCVGGCMAMAKTTLLTEDDGFITAAKLLGESLHKTLTESGGIVKDIEVFEDLFKDGLPTTMIGVAGTPKLKFYETDFGWGNPKKVETISIDYNMSISMNACRESKDDLEIGVCLMNTEMEAFVRLFDEGLESYV

SEQ ID NO: 18 - protein, 442 aa, Helichrysum umbraculigerum
MGSENVHKIMKINITKSSFVQPSKPTVLPTNHIWTSNLDLVVGRIHILTVYFYRPNGASNFFDPIVMKKALADVLVSFYPMAGRISKDDNGRVVINCNDEGVLFVEAESDSTLDDFGEFTPSPELRQLTPTIDYSGDISTYPLFFAQVTHFKCGGVGFGCGVFHTLADGLSSIHFINTWSDMARGLSIAIPPFTDRTLLRAREPPTPTFDHVEYHLPPSMKTTSQTNKSRKPSTAMLKLTLDQLNALKAAAKNEGGNTNYSTYEILAAHLWRCACKARGLPDDQLTKLYVATDGRSRLSPQLPPGYLGNVVFTATPVAKSADLTTQPLSNAASLIRTTLTKMDNDYLRSAIDYLEVQPDLSALIRGPSYFASPNLNINTWTRLPVHDADFGWGRPVFMGPAVILYEGTIYVLPSPNNDRSMSLAVCLDADEQPSFEKFLYDF

SEQ ID NO: 19 - protein, 474 aa, Helichrysum umbraculigerum
MPSSSSSPSSTADSVTIISKCTVYPHMKNSTPESLQLSVSDLPMLSCQYIQKGVLLSQPPPNHTNNIISHLKLSLSKTLSHFPPLAGRLSTDSHGHVSIICNDSGVEFVHSTANHLHTHQILPLNSDVHPCFKTFFAFDKTLSYAGHHQPIAAVQVTELADGLFIGCTVNHAVVDGTSFWNFFNTFAEITKGCQKVTNLPDFSRENVFISPVVLPLPSGGPSATFSGDEPLRERIIHFSRDAILKMKFRANNPLWRQPQNSDLDDTEIYGKVCNDINGKVNGAFKPKSEISSFQSLCGQLWRAVTRARKFNDPIKTTTFRMAVNCRHRLDPKVDKLYFGNLIQSIPTVASVGELLSHDLSWAANELHQNVVAHDNATVRRGVKDWENNPKLFPLGNFDGAMITMGSSPRFPMYNNDFGWGRPMAVRSGKANKFDGKISAFPGRDGDGSVDLEVVLAPETMACLERDHEFMQYVS

SEQ ID NO: 20 - protein, 535 aa, Helichrysum umbraculigerum
MKWFFITHKATQRCLNSKQFHLHGGSNFVSGNRCFLASHSMERPKFMLIPYYPYQIRSLNSSHRYSSTSPSGSPHSFLNGTKNENYTKKVDLEIISREIIKPASPTPHHLRNFNLSLLDQIVFDCYTPVILFIPNSNKATVTDVMIKRLKHLKETLSRILSQFYPFAGEVKDRLHIECNDKGVNYIEAQINETLEEFLCHPDNEKARELMPESPHVQESAIGNYAMGIQINIFSCGGIGLSMSMAHKIMDFYTYTIFMKAWAAAVRGSPDTIISPSFVASEVFPNDPSQEDSIPIELKSSNLLSTKRFEFDPTALALLKGQVVASGSPPQRGPSRMEATTAVIWKAAAKAASTVRRFDPKSPHALALPVNIRKRASPALPDNSIGNIVMRGIAICFPESQPDLPTLMGKVRESIAKLNSDYIESLKGEKGHETVNKMLKELKLRTNMTKVGGKFVASCIFNSGIYELDFGWGKPIWFYVVNPGSDSCVVLTDTLKGGGVEATITLPPDEMEIFERDHELLSYTTINPSPLRFLDH

SEQ ID NO: 21 - protein, 464 aa, Helichrysum umbraculigerum
MEVPDQFHLNILEQCHVSPSPNSIIPSFSLPLTFLDIPWLFYPSNQTLFFFPEPPPKTTIITTLKQSLSLTLHHFHPLAGNLSLPSPPAEPHIVYTKNDSIALTIAQTNTNIHHLSCNHPRSVKNLYSLLPKLPSPSMSRETHVGLVIPLLTIQITVFADLGYSIGVTMQHAAVDERTFDQFMKCWASVCTSLLKNDSLFTFKSTPWYDRSVIIDPKSLKTTFLKQWWNRSNSLNESHDQENDDHDLVLATFVLSSLDINMIKNHILAKCKMINEDPPLHLSPYVSACAYLWKCLIKIQETHDSIKGGPLYLGFNAGGITRLGYDIPSTYFGNCIAFGRCKAFESELLGDNGIVFAAKSIGKEIKRLDKDVLGGANKWISDWDELTIRLLGSPKVDSYGMDFGWGKVEKVEKISSISNHGRVNVISLSGCKDFKGGIEIGVVLSVAKMNVFTSLFHGGLMEFAY

SEQ ID NO: 22 - protein, 405 aa, Helichrysum umbraculigerum
MKNKNPTSVIREALAKVLVFYYPFAGRLKEGPARKLMVDCSGEGVLFIEAEADVTLKQFGDALQPPFPCLEELLYDVPGSTGILDTPLLLIQVTRLLCGGFIFALRLNHTMSDAAGLVQFMTGLGEMAQGASRPSTLPVWQRELLFARDPPRVTCTHHEYTEVEDTNGTIIPLDDMAHKSFFFGPSEISALRRFVPSYLKKCSTFEVLTACLWRCRTIALQPDPEEEMRMICIVNARGKFNPPLLPKGYYGNGFAIPVAISTAGDLSSKPLGHALELVMKAKSNVTEEYMRSVADLMVIKGRPHYTVVRSYLVSDVTHAGFDVVDFGWGKASYGGPAKGGVGAIPGVVTFFIPFTNHKGESGIVLPICLPSAAMDKFVEELNKMLVPDNNEQVLREHKLLVLARL

SEQ ID NO: 23 - protein, 461 aa, Helichrysum umbraculigerum
MAQIDTPLTFKVRRHAPELIAPAKPTPRELKPLSDIDDQEGLRFHIPVIQFYRSDPKMKNKNPASVIREALAKVLVFYYPFAGRLKEGPARKLMVDCSGEGVLFIEAEADVTLKQFGDALQPPFPCLEELLYDVPGSTGVLDTPLLLIQVTRLLCGGFIFALRLNHTMSDAPGLVQFMTGLGEMAQGASRPSTLPVWQRELLLARDPPRVTCTHHEYTEVEDTKGTIIPLDDMAHKSFFFGPSEISALRRFVPSYLKKCSTFEVLTACLWRCRTIALQPDPEEEMRIICIVNARGKFNPPLPKGYYGNGFAFPVAISTAGDLSSKPLGHALELVMKAKSDVTEEYMRSIADLMVIKGRPHFTVVRSYLVSDVTHAGFDVVDFGWGKAAYGGPAKGGVGAIPGVASFYIPFTNHKGESGIVLPICLPSAAMDKFVEELNKMLVPDNNEQVLREHKLLVLARL

SEQ ID NO: 24 - protein, 443 aa, Helichrysum umbraculigerum
MEIQVINYSSKLVKPLTPTPTANRYYNISFTDELVPTIYVPLILYYATPKNPNGDHFENICDRLEESLSKTLSDFYPLAARFIRKLSLIDCNDQGVLFVLGNVNIRLSDVTGLGLTFKTSVLNDFLPCEIGGADEVDDPMLCVKVTTFECGGFAIGMCFSHRLSDMGTMCNFINNWAARTIGEYDNEKHTPIFNSPLYFPQRGLPELDLKVPRSSIGVKNAARMFHFNGKAISSMREVFGVDENGSRRLSKVQLVVALLWKAFVRIDDVNDGQSKASFLIQPVGLRDKVVPPLPSNSFGNFWGLATSQLGPGEGHKIGFQEYFYILRESIKKRARDCAKILTHGEEGYGVVIDPYLESNQKIADNGTNFYLFTCWCKFSFYEADFGCGKPIWASTGKFPVQNLVIMMDDNEGDGVEAWVHLDDKRMNELEQDPDVKLYACNLA

SEQ ID NO: 25 - protein, 434 aa, Helichrysum umbraculigerum
MKLAVKESVIVKPSKTTPCQQIWTSNLDLVVGRIHILTVYLYRPNGSSNFFDSMVLKKALADVLVSFFPVAGRLDKDGDGRVVIDCNGEGVLFVEAEADCCIDDFGEITPSPELRRLVPTVDYSGDMSSYPLFITQVTRFKCGGVSLGCGLHHTLSDGLSALHFINTWSDVARGLSVAIPPFIDRSLLRARDPPSPVFDHIEYHPPPSLITPLQNQKNASHSRSASTLILRLTLHQINNLKSKAKGDGSMYHSTYEILAAHLWRCACKARGLANDQPTKLYVATDGRSRLIPPLPPGYLGNVVFTATPVAKSGDFESESLAETARRIRSELGKMNDEYLRSAIDYLESVSDISTLVRGPTYFASPNLNVNSWTRLPIYESDFGWGRPIFMGPASILYEGTIYIIPSPSGDRSVSLAVCLDPDHMALFKECLYVF

SEQ ID NO: 26 - protein, 434 aa, Helichrysum umbraculigerum
MKLAVKESVIVKPSKTTPCQQIRTSNLDLVAGRIHILVVFFYRPNGSSNFFDSLVLKKALADVLVPFFPVAGRFSEDGDGRVVIDCNGEGVLFVESEADCCIDDFGEITLSPELQQLVPTVDYSGDMSSYPLFIAQVTRFKCGGVSLGWGLHHTLLDGLSALHFVNTWGDVARGLSVAIQPFIDRSLLRARDPPTPVFDHIEYHPPPSLITPLQNQKNASHSRSASTLILQLTPDQIKNLKSKAKGDGSMYHSTYEILAAHLWRCACKARGLANDQPTKLYVAANGRSRLIPPLPPGYLGNVVFNATHVAKSGDFESESLAETARRIHCELGKMNDEYFRSAIDYLESVDDISTLVKGPTYFASPNLNVYSWIGIPIYACDFGWGQPIFMRPASFLYDGSIYIIPSPSGDRSVLLAVCLDPDHMDLFKECLYAF

SEQ ID NO: 27 - protein, 460 aa, Helichrysum umbraculigerum
MVMISKLLRLGRRKLHTIVSRDTIRPSSPTPSHSKTYNLSLLDQIAVNSYVPIVAFYPSSNVCRSSDDKTLELKNSLSKILTHYYPFAGRMKKNRPTVVDCNDEGVEFVEARNTNSLSDFLQQSEHEDLDQLFPDDCVWFKQNLKGSINDANNSSVCPLSIQVNHFACGGVAVATSLRHKIGDGSSALNFIKHWAAVTSHSRAGNHQIDATSPIINPHFISYPTRTFKLPDRSPYIPPSDVVSKSFVFPNTNIKDLQAKVVTMTMGSRQPIVNPTRADVVSWLLHKCVVAAATKRISGNFKESCVISPLNLRNKLEEPLPETSIGNIFYLITFPISNNHGDLMPDDFISQLRLGIRKFQNIRNLETALRTVEEMISETFILGTAESMDTSYVYSSIRGFPMYDIDFGWGKPVKVTVGGALKNLSILMDTPDVNGIEALVSLDKQDMKILLNDPELLAFCL

SEQ ID NO: 28 - protein, 440 aa, Helichrysum umbraculigerum
MSTSDKMKITIRESSMIKPSKPTPDQRIWNSNLDLVVGRIHILTLYFFRPNGSSDFFDSEVLKQSLADVLVSFFPMAGRLGLDGDGRVEINCNGEGVLFVEAEADCSIDDFGEITPSPELRRLAPTVDYSGDISSYPLVITQVTHFKCGGVSLGCGLHHTLSDGLSSLHFINTWSDVTRGLPVAIPPFVDRTVLRARDPPTVVFDHVEYHTPPSMTSSLDKDKPQSEDVHVSTSMLRLTLDQINALKAKGKGDGIVYHSTYEILAAHLWRCACKARGLLNDQMTKLYVATDGRSRLIPPLPPGYLGNVVFTATPIAKSGELQQEPLATTARKIHTELAKMDDKYLRSALDYLESQQDLSALIRGPAYFACPNLNINSWTRLPIYDADFGWGRPIFMGPASILYEGTIYIIPSPSGDRSVSLAVCLDPSHMPLFQKYLYEL

SEQ ID NO: 29 - protein, 441 aa, Helichrysum umbraculigerum
MVNVEIISNEYIKPSSPTPPHLKIYNLSILDQLIPAPYAPIILYYPNQDHINDFEVHERLKLLKDSLSKTLTRFYPLAGTIKGDLSIDCNDIGAYFAVAHVNTRLDVFLNHPDLDLINCFLPRGPYLNGSSEGSCVSNVQVNIFECCGIAISLCISHKILDGAALSTFLKAWAGTSYGSKEVVYPNMSAPSLFPAKDLWLKDSSMVMFGSLFKMGKCSTKRFVFDSSKLSFLKAKASLNGLKDPTRVEVVSALLWKCIMAASEENTGSWKPSLLSHVVNLRKRLVSTLSEDSIGNLIWLASAECRTNAQSRLSDLVEKVRDSVSKINSEFVKKIQGDKGTKVMEESLKSMKDCADYIGFTSWCKMGFYDVDFGWGKPVWVCGSVCEGSPVFMNFVILMDTKYGDGIEAWVSLDEHEMHILKHNPELLEYASIDPSPLQMNK

SEQ ID NO: 30 - protein, 454 aa, Helichrysum umbraculigerum
MGTIYQSPMIKSSTPKIIEDLKVIIHDTFTIFPPHETEKRSMFLSNIDQVLTFNVETVHFFAANPDFPPQVVAEKLKLALSKALVPYDFLAGRLKLNHESQRFEFDCNGAGARFVVGSSEFELGEIGDLVYPNPGFRQLVQKSYDNLELHEKPLCILQLTSFKCGGFALGVATNHATFDGLSFKTFLQNLGSLAADQPLAVDPCNDRHLLAARSPPKVQFDHPELLKIPTGTDIPNPTVFDCPESQLDFKIFNLTSDDIAHLKTKAKDGPGSTNAKITGFNVVAAHVWRCKALSSGSEYDPERVSTVLYAVDIRSRLNLPLSLAGNAVLSAYASAKCKEIEEGPLSRLVEMVTEGTNRMTGEYARSVIDWGEVNKGFPNGEFLISSWWRLGFADVEYPWGKPRYSCPVVYHRKDIILLFPDIVGADNNNEVNVLVALPGKEMEKFETLFHKFLA

(19) United States
(12) Patent Application Publication
(10) Pub. No.: US 2025/0327043 A1
(43) Pub. Date: Oct. 23, 2025

(54) ALCOHOL ACYLTRANSFERASE AND A TRANSGENIC CELL, TISSUE, AND ORGANISM COMPRISING SAME

(71) Applicant: YEDA RESEARCH AND DEVELOPMENT CO. LTD., Rehovot (IL)

(72) Inventors: Asaph AHARONI, Tel Aviv (IL); Paula BERMAN, Rehovot (IL); Luis DE-HARO, Rehovot (IL); Adam JOZWIAK, Rehovot (IL)

(21) Appl. No.: 18/855,903

(22) PCT Filed: Apr. 13, 2023
(86) PCT No.: PCT/IL2023/050393

§ 371 Date: Oct. 10, 2024

Related U.S. Application Data
(60) Provisional application No. 63/330,527, filed on Apr. 13, 2022.

(51) Int. Cl. C12N 9/10; C12N 15/82; C12P 7/42; C12P 7/62

(52) U.S. Cl. CPC C12N 9/1029; C12N 15/8243; C12P 7/42; C12P 7/62; C12Y 203/01084

(57) ABSTRACT

The present invention provides polynucleotide sequences derived from Helichrysum umbraculigerum and encoding a protein or a plurality thereof belonging to the alcohol acyltransferase (AAT) family. Further provided are an artificial nucleic acid molecule including the polynucleotide disclosed herein, a transgenic cell, tissue, or plant including same.

### CROSS-REFERENCE TO RELATED APPLICATIONS

This application claims the benefit of priority of U.S. Provisional Patent Application No. 63/330,527, titled “ALCOHOL ACYLTRANSFERASE AND A TRANSGENIC CELL, TISSUE, AND ORGANISM COMPRISING SAME”, filed Apr. 13, 2022. The contents of which are all incorporated herein by reference in their entirety.

### REFERENCE TO AN ELECTRONIC SEQUENCE LISTING

The contents of the electronic sequence listing (YEDA-P-014-PCT ST26.xml; size: 54,156 bytes; and date of creation: Apr. 13, 2023) is herein incorporated by reference in its entirety.

### FIELD OF INVENTION

The present invention relates to alcohol acyltransferases (AAT) and a transgenic cell, tissue, and organism comprising same including polynucleotides encoding same, and methods of using same, such as for producing acylated cannabinoids.

### BACKGROUND

In the past few years, the therapeutic usage of cannabinoids has made a significant leap as new reports highlight their potential for various medical purposes. Cannabinoids have been found to exert diverse biological and pharmacological effects via modulation of the metabotropic cannabinoid receptors CB₁ and CB₂, the ionotropic thermo-TRP ion channels, and the transcription factors from the PPAR family. Cannabinoids are typical of Cannabis sativa L. (Cannabis), although some specific compounds have also been identified in other flowering plants, liverworts, and fungi. One of these plants is Helichrysum umbraculigerum Less (Helichrysum). This perennial South-African plant is the only known plant other than Cannabis, producing cannabigerolic acid (CBGA), the five-carbon alkyl precursor of all the major cannabinoids. This plant has also been suggested as the most known versatile source of cannabinoids, since, along with CBGA, it also produces different aralkyl-type cannabinoids denoted as amorfrutins. Previous reports identified in Helichrysum isoprenylated O-acylated compounds from the aralkyl-type, which are unique to this plant. Among these, O-methylbutyryl deprenylhelycannabigenol and its acid counterpart were assayed against CB₁, CB₂, and the thermo-TRP targets of cannabinoids, and the neutral acylated amorfrutin was found to exhibit sub-micromolar affinity for the ionotropic TRPV₃ ion channel8. In addition, the acid acylated amorfrutins were found to be stable under the decarboxylation conditions of acid cannabinoids. Several recent studies report on the pharmacological activities of some acid cannabinoids. However, their poor thermal stability impede their development as new therapies. Acylation of cannabinoids might therefore be a possible solution for producing more stable treatments based on acid cannabinoids, providing that their pharmacological activities are not hindered by the acylation.

Despite the impressive therapeutic potential of this group of compounds, the enzymes catalyzing the reaction in this plant have not been identified. O-Acylation of aromatic specialized metabolites (SMs) in plants is catalyzed either by BAHD (named according to the first letter of each of the first four biochemically characterized enzymes of this family: BEAT, AHCT, HCBT and DAT) or serine carboxypeptidase-like (SCPL) acyltransferases. These two families of acyltransferases differ by the energy-rich acyl donor that they utilize: BAHD enzymes use activated acyl-CoA thioesters, while SCPLs use 1-O-β-glucose esters. Numerous acyltransferases that acylate aromatic SMs have been identified in different plants, including hydroxybenzoic acids, hydroxycinnamic acids, flavonoids, and more. However, no acyltransferases have been identified which acylate cannabinoids yet.

Therefore, there is a need for developing methodologies that allow large-scale acylation of cannabinoids, which may increase their processability and/or applicability.

### SUMMARY

The present invention, in some embodiments, is based, in part, on the identification of acylated forms of CBGA-type cannabinoids and geranylated O-acylated amorfrutins. In addition, the inventors identified two novel BAHD alcohol acyltransferases (AATs) that catalyze the acylation of the naturally occurring and some additional unique cannabinoids, thus, paving the way to potential new modulators of the endocannabinoid system.

According to a first aspect, there is provided an isolated DNA molecule comprising a nucleic acid sequence having at least 87% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or any combination thereof.

According to another aspect, there is provided an artificial nucleic acid molecule comprising the isolated DNA molecule disclosed herein.

According to another aspect, there is provided a plasmid or an agrobacterium comprising the artificial nucleic acid molecule disclosed herein.

According to another aspect, an isolated protein encoded by any one of: (a) the isolated DNA molecule disclosed herein; (b) the artificial vector disclosed herein; and (c) the plasmid or agrobacterium disclosed herein.

According to another aspect, there is provided a transgenic cell comprising: (a) the isolated DNA molecule disclosed herein; (b) the artificial nucleic acid molecule disclosed herein; (c) the plasmid or agrobacterium disclosed herein; (d) the isolated protein disclosed herein; or (e) any combination of (a) to (d).

According to another aspect, there is provided an extract derived from the transgenic cell disclosed herein, or any fraction thereof.

According to another aspect, there is provided a transgenic plant, a transgenic plant tissue or a plant part, comprising: (a) the isolated DNA molecule disclosed herein; (b) the artificial vector disclosed herein; (c) the plasmid or agrobacterium disclosed herein; (d) the isolated protein disclosed herein; (e) the transgenic cell disclosed herein; or (f) any combination of (a) to (e).

According to another aspect, there is provided a composition comprising: (a) the isolated DNA molecule disclosed herein; (b) the artificial vector disclosed herein; (c) the plasmid or agrobacterium disclosed herein; (d) the isolated protein disclosed herein; (e) the transgenic cell disclosed herein; (f) the extract disclosed herein; (g) the transgenic plant tissue or plant part disclosed herein; or (h) any combination of (a) to (g), and an acceptable carrier.

According to another aspect, there is provided a method for acylating a cannabinoid comprising: (a) providing a cell comprising an artificial vector comprising a nucleic acid sequence having at least 87% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15; and (b) culturing the cell from step (a) such that a protein encoded by the artificial vector is expressed, thereby acylating the cannabinoid.

According to another aspect, there is provided an extract of a cell obtained according to the method disclosed herein.

According to another aspect, there is provided a medium or a portion thereof separated from a cultured cell, obtained according to the method disclosed herein.

According to another aspect, there is provided a composition comprising: (a) the extract disclosed herein; (b) the medium or a portion thereof disclosed herein; or (c) a combination of (a) and (b), and an acceptable carrier.

According to another aspect, there is provided a method for acylating a cannabinoid, the method comprising contacting the cannabinoid or precursor thereof with an effective amount of a protein comprising an amino acid sequence with at least 91% homology to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, thereby acylating the cannabinoid.

In some embodiments, the nucleic acid sequence has at least 87% homology to any one of SEQ ID Nos.: 1-15 is 1,00 to 1,800 nucleotides long.

In some embodiments, the nucleic acid sequence encodes a protein being an alcohol acyltransferase (AAT).

In some embodiments, the isolated protein comprises an amino acid sequence with at least 91% homology to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30.

In some embodiments, the isolated protein consists of an amino acid sequence of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30.

In some embodiments, the isolated protein is characterized by being capable of acylating a cannabinoid.

In some embodiments, the transgenic cell is any one of: a unicellular organism, a cell of a multicellular organism, and a cell in a culture.

In some embodiments, the unicellular organism comprises a fungus or a bacterium.

In some embodiments, the fungus is a yeast cell.

In some embodiments, the extract comprises the isolated DNA molecule, the isolated protein, or both.

In some embodiments, the transgenic plant is a Cannabis sativa plant.

In some embodiments, the cell is a transgenic cell, or a cell transfected with the isolated DNA molecule disclosed herein or the artificial vector disclosed herein.

In some embodiments, the protein is characterized by being capable of transferring an acyl group from a donor molecule to the cannabinoid.

In some embodiments, the culturing comprises supplementing the cell with an effective amount of a donor molecule comprising an acyl group.

In some embodiments, the artificial vector is an expression vector.

In some embodiments, the cell is a prokaryote cell or a eukaryote cell.

In some embodiments, the method further comprises a step (c) comprising extracting the cell, thereby obtaining an extract of the cell.

In some embodiments, the method further comprises a step preceding step (c), comprising separating the cultured cell from a medium wherein the cell is cultured.

In some embodiments, the method further comprises a step preceding step (a), comprising introducing or transfecting the cell with the artificial vector.

In some embodiments, contacting is in a cell-free system.

In some embodiments, the cannabinoid is CBGA, heliCBGA, CBDA, or any combination thereof.

In some embodiments, the cannabinoid is acylated at one or more functional group thereof, being selected from the group consisting of: O, OH, N, NH, NH₂, and any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B include graphs showing identification of CBGA and heliCBGA in a Helichrysum ethanolic extract. Extracted ion current (XIC) chromatograms and MS/MS spectral matching of (1A) cannabigerolic acid (CBGA, 359.222 Da) and (1B) helicannabigerolic acid (heliCBGA, 393.206 Da), standards or authentic compounds versus a Helichrysum sample.

FIGS. 2A-2F include chemical structure elucidations and graphs showing stable isotope labeling of CBGA (C1) and heliCBGA (A1) via feeding of Helichrysum leaves with hexanoic-D₁₁ acid, or phenylalanine-D₅ and phenylalanine-¹³C₉, respectively. Helichrysum leaves were fed with either double distilled water (DDW, control), (2A) unlabeled/labeled hexanoic acid, or (2D) unlabeled/labeled phenylalanine for three days, then cannabinoids were extracted and analyzed via UPLC-qTOF. (2B-2E) Extracted ion current (XIC) chromatograms and (2C-2F) MS/MS spectra of CBGA and heliCBGA and their corresponding labeled peaks. The MS/MS spectra of the non-labeled versus the labeled forms show similar fragmentation patterns with mass shifts corresponding with the labeling. Since labeled metabolites possess nearly identical physicochemical properties as their native non-labeled analogues, the newly derived iso-topologues were detected as co-eluting chromatographic peaks (unlabeled and labeled forms), except that their m/z values were different.

FIGS. 3A-3L include chemical structure elucidations and graphs showing identification of O-acylated cannabinoids. (3A-3J) MS/MS spectra in negative polarity of unlabeled and isotopically labeled O-acylated cannabinoids (C2-C14). The compounds were identified by specific fragmentation patterns as exemplified for O-MeButCBGA (3K) according to MS/MS spectra and labeling [the structure of O-MeButCBGA was confirmed via NMR (FIG. 6)]. As shown, the position of the FA, either as the alkyl tail or acyl group can be deduced from the MS/MS fragmentation spectra. Fragments colored in blue or red correspond to the m/z of the specific fragment with labeled alkyl chain or acyl group, respectively. Also, the isoprenylated compounds exhibited analogous fragmentation patterns to the geranylated ones, with mass shifts corresponding with one prenyl group (m/z difference of 68.063). In addition, the isoprenylated compounds eluted several minutes before the geranylated ones, as a result of increasing lipoliphicity with prenylation, and the relative order of elution was in relation to FA chain length (increasing alkyl chain length→longer retention times as a result of increasing lipophilicities). Compounds C2-C12 had five-carbon tails (according to labeling with hexanoic-D₁₁ acid) whereas compounds C13-C14 had six-carbon tails (3J). (3L) Structures of the observed compounds and isotopically labeled precursors of the identified compounds; IP, isoprenyl; MP, monoprenyl.

FIGS. 4A-4C include chemical structure elucidation and graphs showing identification of hydroxylated and dihydroxylated O-acylated cannabinoids. (4A) MS/MS spectra in negative polarity of hydroxylated (C15-C16) and dihydroxylated (C17-C19) cannabinoids. The MS/MS spectrum of C9 is shown as reference. The labeled compounds were not observed probably due to low abundance in the extracts, however, according to the observed fragmentation patterns the inventors putatively assigned the structures presented in 4B with the addition of one or two hydroxyls at the marked possible positions. (4C) Representative suggested fragmentation structure of C15 according to its observed MS/MS spectrum in relation to the elucidated fragmentation structure of C9 (FIG. 3K).

FIGS. 5A-5H include chemical structure elucidations and graphs showing identification of O-acylated amorfrutins. (5A-5F) MS/MS spectra in negative polarity of unlabeled and isotopically labeled O-acylated amorfrutins (A2-A12). The compounds were identified by specific fragmentation patterns as exemplified for O-MeButheliCBGA (5G) according to MS/MS spectra and labeling [the structure of O-MeButheliCBGA was confirmed via NMR (FIG. 7)], and by following the same fragmentation patterns and relative RTs observed for cannabinoids (FIG. 3). (5H) Structures of the observed compounds and isotopically labeled precursors of the identified compounds; IP, isoprenyl; MP, monoprenyl; fragments colored in blue, green or red correspond to the m/z of the specific fragment in the compound labeled with phenylalanine-D₅, phenylalanine-¹³C₉ or the corresponding fatty acid, respectively.

FIGS. 6A-6C include a table and chemical structure elucidation of O-MeButCBGA (C9) via 1D and 2D NMR. (6A) ¹H and ¹³C chemical shift assignment, (6B) atom numbering and COSY correlations, and (6C) HMBC correlations of O-MeButCBGA. The carbon on the carboxyl was not observed in the NMR spectra, however, UPLC-qTOF spectra and chemical formula confirm the presence of this group.

FIGS. 7A-7C include a table and chemical structure elucidation of O-MeButheliCBGA (A9) via 1D and 2D NMR. (7A) ¹H and ¹³C chemical shift assignment, (7B) atom numbering and COSY correlations, and (7C) HMBC correlations of O-MeButheliCBGA. The carbon on the carboxyl was not observed in the NMR spectra, however, UPLC-qTOF spectra and chemical formula confirm the presence of this group.

FIGS. 8A-8G include a graph and micrographs showing CBGA and O-MeButCBGA content in plant tissues and localization of CBGA to glandular trichomes of Helichrysum leaves and flowers. (8A) CBGA absolute concentrations (% w/w, n=4) and peak areas of O-MeButCBGA (according to UPLC-qTOF peak areas of m/z of 443.28) in different Helichrysum plant tissues. Optical images (8B and 8D) and MALDI-MSI (8C and 8E) of m/z 361.24±0.01 Da (corresponding with the protonated mass of CBGA) of a cross sectioned leaf and a flower receptacle. Trichomes in 8B and 8D are marked to improve interpretation. CBGA is localized to stalked glandular trichomes. Optical images of the (8F) flower head and (8G) cut receptacle for reference. Stalked glandular (yellow arrow) and mechanical trichomes (white arrow) can be observed on the surface border of the tissue in (8G). The white broken lines in (8C) and (8E) mark the regions analyzed. Scale bar: 100 μm (8B); 500 μm (8C); 200 μm (8D); 1,000 μm (8E); 1,000 μm (8F); and 500 μm (8G).

FIGS. 9A-9C include graphs showing expression profiling of Helichrysum genes (UMI-aware 3′ Trans-seq). (9A) PCA plot of the normalized gene expression distribution of the 21 samples sequenced. (9B) Over-representation analysis (ORA) of groups of genes belonging to each coexpression module obtained with CEMI-tools. Module number M4 includes genes enriched in trichomes and in leaves. (9C) Normalized expression of the genes belonging to the module number 4.

FIG. 10 include a graph showing expression profiles of selected AAT Helichrysum genes (UMI-aware 3′ Trans-seq). CPM normalized expression of selected AAT genes with expression patterns correlated with CBGA accumulation. A secondary axis including CBGA quantification is included in the right side of the plot.

FIG. 11 includes curves showing activities of lysates containing HuAATs with butyryl-and hexanoyl-CoA as the acyl donors, and CBGA and heliCBGA as the acceptors. All LC/MS chromatograms were selected for the theoretical m/z values of the respective compounds of interest. Samples were analyzed with a short 20 min multistep gradient method: initial conditions were 40% B for 1 min, raised to 100% B until 14 min, held at 100% B for 3.8 min, decreased to 40% B until 18 min, and held at 40% B until 20 min for re-equilibration of the system. Only HuAAT5 and HuAAT14 (red and blue, respectively) acylated the cannabinoids with both acyl-CoAs. EV, empty vector.

FIG. 12 includes a phylogenetic analysis of AAT genes from different plant species. The Maximum Likelihood tree was constructed with 100 bootstrap tests based on a MUSCLE multiple alignment using the MEGA11 software. The evolutionary distances were computed using the JTTmatrix-based method. Bootstrap values are indicated at the nodes of each branch. Phylogenetic clades are numbered based on Tuominen et al., 2011. HuAAT genes are highlighted in red. The active HuAAT5 and HuAAT14 were clustered in clade IIIa which represents BAHDs of diverse catalytic functions.

FIGS. 13A-13B include LC/MS/MS chromatograms and spectra of the acylated cannabinoids following enzymatic assays with the purified HuAAT5 in the presence of acetyl-CoA, iso-butyryl-CoA, butyryl-CoA, iso-valeryl-CoA or hexanoyl-CoA as the acyl donors, and OA, CBGA, heliCBGA, CBDA, Δ⁹-THCA or CBCA as the acceptors. All LC/MS/MS chromatograms were selected for the theoretical m/z values of the respective compounds of interest (cannabinoid acceptors without CoAs: OA>179.107, CBGA, CBCA>191.107, heliCBGA>225.092, CBDA, Δ⁹-THCA>245.154; acylated cannabinoids: OA>179.107, CBGA>231.102, heliCBGA>265.086, CBDA>245.154, Δ⁹-THCA>245.154, CBCA>191.107). (13A) The detected analog peaks were shifted in retention time depending on their change in hydrophobicity relative to the cannabinoid acceptor. (13B) Identification was according to specific MS/MS fragmentation patterns. Similar fragments are marked for each substrate.

### DETAILED DESCRIPTION

The present invention, in some embodiments, is directed to polynucleotide sequences derived from Helichrysum umbraculigerum and encoding a protein or a plurality thereof belonging to the alcohol acyltransferase (AAT) family.

According to some embodiments, there is provided a polynucleotide comprising a nucleic acid sequence comprising any one of SEQ ID Nos.: 1-15, or any combination thereof.

In some embodiments, the polynucleotide is an isolated polynucleotide. In some embodiments, the polynucleotide is a DNA molecule. In some embodiments, the polynucleotide is an isolated DNA molecule. In some embodiments, the DNA molecule is an isolated DNA molecule. In some embodiments, the DNA molecule is a complementary DNA (cDNA) molecule.

As used herein, the terms “isolated polynucleotide” and “isolated DNA molecule” refers to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art and may be performed, for example, by ligating or covalently linking by primer linkers multiple nucleic acid molecules together.

The term “nucleic acid” is well known in the art of molecular biology. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

In another aspect of the invention, there is provided a compound, wherein the compound is or comprises an acylated cannabinoid. In some embodiments, the compound of the invention is an isolated compound. In some embodiments, the compound of the invention is a natural or a synthetic compound. In some embodiments, the compound of the invention is a single compound or a plurality of chemically distinct compounds.

In some embodiments, the compound of the invention is chemically pure (e.g., being substantially devoid of one or more impurity, wherein the impurity comprises any organic compound). In some embodiments, the compound of the invention is characterized by a chemical purity of at least 70%, at least 80%, at least 90%, at least 93%, at least 94%, at least 95%, at least 96%, at least 97%, at least 98%, at least 99%, including any range between. In some embodiments, the compound of the invention is characterized by a chemical purity of at most 99.99%, at most 99.9%, at most 99%, at most 95%, at most 90%, including any range between.

As used herein, the term “isolated compound” refers to a compound that is essentially free from contaminating cellular components, such as carbohydrate, lipid, or other proteinaceous impurities associated with the nucleic acid in nature. Typically, a preparation of an isolated compound contains the compound in a highly purified form, e.g., at least about 80% pure, at least about 90% pure, at least about 95% pure, greater than 95% pure, or greater than 99% pure.

In some embodiments, the compound of the invention is represented by Formula 1:

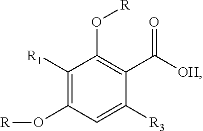

including any salt, any tautomer, any stereoisomer and/or a decarboxylated derivative thereof, wherein:
R1 is

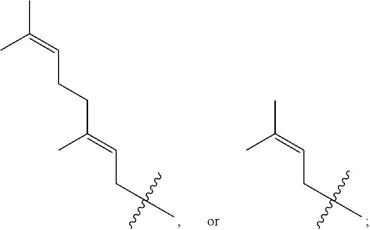

each R is independently H or

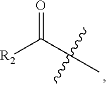

and wherein any one of R3 and R2 independently comprises an alkyl (a linear or a branched alkyl), optionally comprising one or more unsaturated bonds; and wherein at least one R is

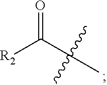

### or by Formula I:

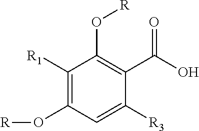

wherein R1 is

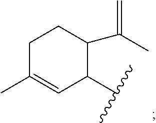 [SMILES: *C1C=C(C)CCC1C(=C)C]

R and R′ are independently H or

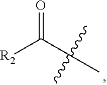

and wherein any one of R3 and R2 independently comprises an alkyl (a linear or a branched alkyl), optionally comprising one or more unsaturated bonds; and wherein R or R′ is

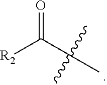

In some embodiments, the compound of the invention is represented by Formula:

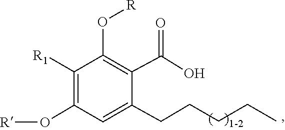

wherein R1 is

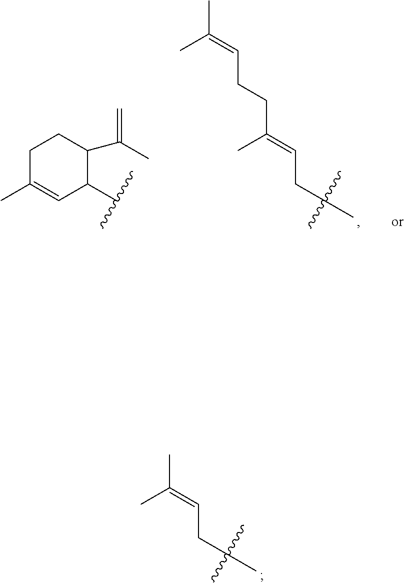

and R and R′ are as described hereinabove.

In some embodiments, R2 is or comprises H or an alkyl, wherein the alkyl comprises between 1 and 30, between 1 and 10, between 1 and 20, between 1 and 22, between 1 and 18, between 1 and 3, between 1 and 4, between 1 and 5, between 1 and 6, between 5 and 10, between 5 and 30, between 5 and 22, between 3 and 30, between 3 and 10, between 3 and 22, between 10 and 30, between 10 and 20, between 10 and 22, between 20 and 30, between 1 and 25, between 22 and 30 carbon atoms, including any range between. In some embodiments, R2 is or comprises an alkyl chain of a naturally occurring fatty acid (e.g., C1-C22 fatty acid). In some embodiments, R2 is or comprises a linear or a branched alkyl.

In some embodiments, the compound of the invention is represented by Formula 1A:

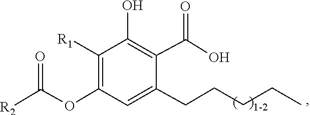

wherein R1 and R2 are as described hereinabove.

In some embodiments, R2 is or comprises any of

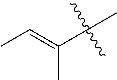 [SMILES: */C(C)=C/C]

(1-methyl-1-propenyl), iso-butyl, sec-butyl, propyl, iso-propyl, butyl, butylene, and pentyl.

In some embodiments, the compound of the invention is represented by any of Formulae 1 or 1A, wherein R1 is

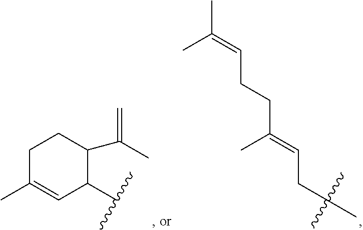 [SMILES: *C1C=C(C)CCC1C(=C)C ; *C/C=C(\C)CCC=C(C)C]

and R2 is selected from iso-butyl, sec-butyl, propyl, iso-propyl, butyl, butylene, 1-methyl-1-propenyl, and pentyl.

In some embodiments, the compound of the invention is represented by any of Formulae 1 or 1A, wherein R1 is

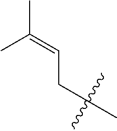 [SMILES: *CC=C(C)C]

and R2 is selected from propyl, sec-butyl, butyl, 1-methyl-1-propenyl, and butylene.

In some embodiments, the compound of the invention is represented by any of Formulae 1 or 1A, wherein R1 is any of

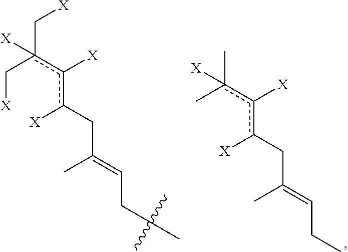

wherein [symbol] is a single bond or a double bond; wherein each X independently represents H and OH, and wherein at least one X is OH. In some embodiments, the compound of the invention is represented by any of Formulae 1 or 1A, wherein R1 is any of:

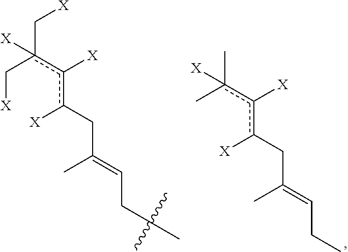

wherein one or two X are OH.

In some embodiments, the compound of the invention is or comprises

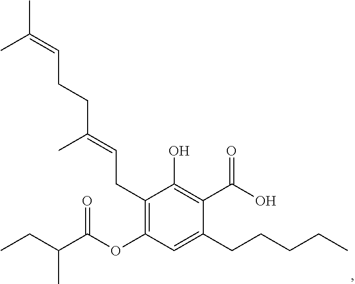 [SMILES: CCCCCc1cc(OC(=O)C(C)CC)c(C/C=C(\C)CCC=C(C)C)c(O)c1C(=O)O]

including any salt and/or a decarboxylated derivate thereof.

In some embodiments, the compound of the invention is represented by Formula 2:

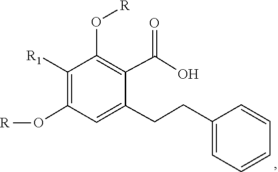

including any salt and/or a decarboxylated derivative thereof; wherein: each R is independently H or

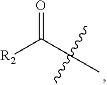

and wherein R2 is or comprises an alkyl, optionally comprising one or more unsaturated bonds, or

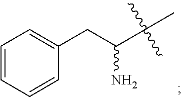 [SMILES: *C(N)Cc1ccccc1]

### and R1 is

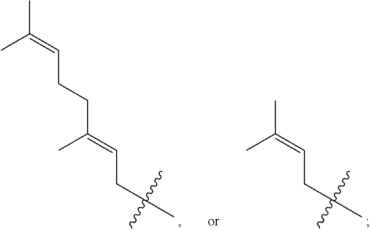

and wherein at least one R is

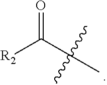

In some embodiments, R2 is or comprises H or an alkyl, wherein the alkyl comprises between 1 and 30, between 1 and 10, between 1 and 20, between 1 and 22, between 1 and 18, between 1 and 3, between 1 and 4, between 1 and 5, between 1 and 6, between 5 and 10, between 5 and 30, between 5 and 22, between 3 and 30, between 3 and 10, between 3 and 22, between 10 and 30, between 10 and 20, between 10 and 22, between 20 and 30, between 1 and 25, between 22 and 30 carbon atoms, including any range between. In some embodiments, R2 is or comprises an alkyl chain of a naturally occurring fatty acid (e.g., C1-C22 fatty acid). In some embodiments, R2 is or comprises a linear or a branched alkyl.

In some embodiments, R2 is or comprises any of

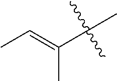 [SMILES: */C(C)=C/C]

(1-methyl-1-propenyl), iso-butyl, sec-butyl, propyl, butyl, butylene, and pentyl.

In some embodiments, the compound of the invention is represented by Formula 2A:

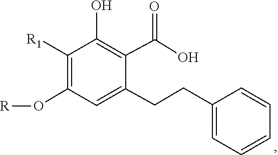

wherein R1 and R are as described herein.

In some embodiments, the compound of the invention is or comprises

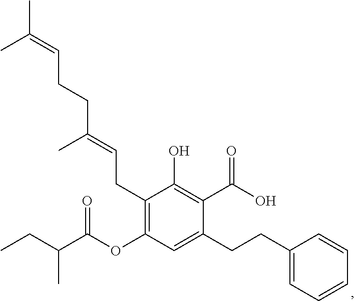 [SMILES: CCC(C)C(=O)Oc1cc(CCc2ccccc2)c(C(=O)O)c(O)c1C/C=C(\C)CCC=C(C)C]

including any salt and/or a decarboxylated derivate thereof.

In some embodiments, the polynucleotide comprises or consists of the nucleic acid

(SEQ ID NO: 1)
ATGGCAACCCAAGTCAAAACCGAGGAGAAGCATTTGAAGGTAGAAATCATAAA
CAAAACCTATGTGAAACCTGAAACACCACTAGGAAGAAAAGAGTGTCAATTGG
TCACATTTGATCTTCCTTATATAGCCTTCTACTACAACCAAAAGTTGATCATCTA
TAAAGGTGGTGTCGAGGAGTTCGAGGATACCGTCGAGAAACTGAAAGACGGGT
TAAAGGTAGTTTTGGGAGAGTTTCATCAATTGGCTGGAAAATTAGACAAAGATG
ATGACGGGGTGTTTAAGGTAGTGTACGATGATGACATGGATGGGGTGGAGGTG
CTTTCTGCGGTCGCGGAAGACACTGCGACCGCAGATTTGATGGACGAAGAAGG
GACCATCAAGCTTAAGGAGTTGGTCCCTTATAATAGTGTTTTGAACATAGAGGG
GCTTCATCGTCCGCTTTTATCGATTCAGATAACAAAACTAAAAGATGGGCTTGT
ACTGGGCTGTGCGTTCAACCACGCGATTTTAGACGGTACATCCACCTGGCACTT
CATGAGCTCCTGGGCCCAAATTTGCTCCGGATCCAAATCCATTTCAGCGGCGCC
TTTCCTTGACCGTACCCAAGCGCGTAACACGCGCGTGAAACTCGATCTCACCCC
TCCCGCCCAAACTAACGGCAATTCAAACGGCGACACTAACGGTGATGCGAGCG
CCACGAAGCCACCAGCACCGGCACCGTTAAGAGAAAAAATCTTCAAATTCTCA
GAGTCAGCAATCGACAAAATCAAAGCAAAAATCAATGCGAATCCACCGGAAGG
ATCAACCAAGCCATTCTCCACATTTCAATCGCTCTCCACACACATATGGCACGC
AGTTACACGCGCTCGCAATCTAAAACCGGAAGACTACACCGTTTTCACTGTTTT
CGCCGATTGCCGGAAACGTGTCGATCCTCCGATGCCGGATAGCTATTTCGGAAA
CCTAATTCAAGCGATCTTCACCGTCACCGCTGCCGGATTATTGCAGGCGAATCC
ACCGGAATTCGCGGCGTCAATGATACAAAAAGCGATTGATATGCACGATGCGA
AAGCAATTGAAGCGCGTAACAAAGAATGGGAAAGTAATCCGATTATATTTCAA
TACAAAGACGCCGGAGTTAATTGTGTTGCGGTTGGGAGTTCACCTAGGTTTAAG
GTTTATGATGTGGATTTCGGGTTTGGTAAACCCGAAAGTGTTCGGAGCGGGGCG
AATAACCGGTTTGATGGTATGGTTTATTTGTATCAGGGAAAAAGTGGTGGAAGG
AGTATTGATGTGGAGATTAGTTTGGATGCAAGTGCAATGGGAAATCTTGAAAA
GGATAAGGAATTTCTTATCCAAGAATAA.

In some embodiments, the polynucleotide comprises a nucleic acid sequence with at least 84%, at least 87%, at least 90%, at least 95%, or at least 99% homology or identity to SEQ ID NO: 1, or any value and range therebetween. Each possibility represents a separate embodiment of the invention. In some embodiments, the polynucleotide comprises a nucleic acid sequence with 84% to 100%, 88% to 100%, 90% to 100%, or 95% to 100% homology or identity to SEQ ID NO: 1. Each possibility represents a separate embodiment of the invention.

In some embodiments, the polynucleotide comprises or consists of the nucleic acid

(SEQ ID NO: 2)
ATGGCTTCTCTTCCTCTCTTAACTGTTCTTGAACAATCCCATGTATCACCACCGC
CAGCCACCGTAGTCGATAAATCGTTGTCGCTAACCTTTTTCGATTTCCTGTGGCT
AACTCAACCTCCAATTCACAATCTTTTCTTTTACGAGTTTTCAATCGACGAAACT
CAGTTCGTGGAAACTATCGTTCCTAGTCTTAAAAACTCGTTATCAATCACTCTTC
AACATTTTTACCCGTTCGCCGGTAACCTTATCTTATTTCCTGATAACAAAAGGCC
TGAAATTCGTTACGTTGAAGGTGATTATGTCATGGTTACATTTGCAAAATCTAG
CCTTGACTTCAATGAACTAGTAGGAAACCATCCTAGAGATTGTGACCAGTTTTA
TGATCTTATTCCTCCATTAGGTGAAAGTGTGAAAACTTCTGAATTTCGAAAAAT
CCCACTCTTTTCGGTCCAGGTGACGTTTTTTCCACAAAAAGGCGTATCGATTGGT
ATGACGAATCATCATAGTCTTGGCGATGCTAGCACTCGGTTTTGTTTCTTGAACG
CGTGGACATCGATTTCTAGATCTAGTTCAGATGAGTCATTTCTAGCAAACGGAA
CTAAACCGTTTTACGATAGAGTGATAAGTAACCCGAAACTAGATCAAAGTTATC
TAAAATTTTCCAAGATCGATACTCTTTACGAGAAGTATCAACCTTTAAGCCTCTC
TAGACCATCTAATAAACTTCGTGGCACGTTTATCTTGACGCGAAAAATCCTAAA
CGAGTTGAAAAAAAGTGTGTCAATTAAACTACCAACTTTATCATATGTATCATC
TTTTACGGTTGCATGTGGTTATATTTGGAGTTGCATAGCGAAATCACGAAACGA
TGATCTACAACTATTCGGGTTCACTATTGATTGTAGGGCACGTTTGGATCCACC
GGTTCCATCAACTTATTTTGGGAATTGTGTCGGGGGTTGTATGGCGATGGCAAA
AACAACGTTGTTAACCGAAGACGATGGATTTATAACGGCTGCTAAATTGCTTGG
AGAAAGTTTACACAAGACGTTGACCGAATCGGGTGGAATCGTGAAAGATATAG
AAGTGTTTGAAGATTTGTTTAAGGATGGATTACCAACAACTATGATAGGAGTTG
CGGGAACACCAAAGCTTAAGTTTTATGAGACGGATTTCGGGTGGGGGAACCCG
AAAAAGGTGGAAACGATTTCGATTGATTATAACATGTCGATTTCTATGAACGCT
TGTAGAGAATCGAAGGATGATTTGGAGATTGGTGTTTGCCTTATGAATACTGAA
ATGGAAGCTTTTGTTCGTTTATTTGATGAAGGATTAGAATCATACGTTTAG.

In some embodiments, the polynucleotide comprises a nucleic acid sequence with at least 77%, at least 85%, at least 93%, at least 97%, or at least 99% homology or identity to SEQ ID NO: 2, or any value and range therebetween. Each possibility represents a separate embodiment of the invention. In some embodiments, the polynucleotide comprises a nucleic acid sequence with 77% to 100%, 80% to 100%, 85% to 100%, or 93% to 100% homology or identity to SEQ ID NO: 2. Each possibility represents a separate embodiment of the invention.

In some embodiments, the polynucleotide comprises or consists of the nucleic acid sequence:

(SEQ ID NO: 3)
ATGGGAAGTGAAAATGTTCACAAAATAATGAAAATCAACATCACTAAATCATC
ATTTGTACAACCCTCAAAGCCTACAGTACTACCCACTAACCACATATGGACTTC
TAACTTAGATTTAGTTGTGGGTAGAATTCATATTTTAACCGTTTACTTTTACCGT
CCAAATGGTGCTTCGAATTTTTTTGATCCAATTGTTATGAAAAAAGCTTTAGCTG
ATGTGCTTGTTTCTTTTTATCCGATGGCCGGAAGAATAAGTAAAGATGATAATG
GTAGAGTTGTAATTAATTGTAATGATGAAGGTGTTTTGTTTGTTGAAGCTGAGT
CAGATTCCACGTTGGATGACTTCGGTGAGTTTACACCGTCTCCGGAGCTCCGAC
AACTTACCCCGACGATTGATTACTCCGGTGACATTTCAACGTACCCGCTATTTTT
TGCACAGGTAACGCATTTCAAGTGTGGAGGAGTTGGTTTTGGTTGTGGTGTGTT
TCATACACTTGCAGATGGTCTATCCTCTATACATTTCATCAACACATGGTCGGAC
ATGGCTCGTGGTCTCTCGATAGCCATCCCGCCATTCACTGACCGGACCCTTCTTC
GTGCACGTGAACCACCCACTCCCACTTTTGACCACGTAGAGTACCACCTCCCTC
CGTCCATGAAAACTACCTCACAAACCAACAAATCCAGAAAGCCTTCCACGGCC
ATGTTAAAGCTTACGCTTGATCAACTAAATGCTCTCAAAGCTGCTGCTAAGAAT
GAAGGCGGCAACACCAATTATAGCACGTACGAGATCCTGGCGGCTCATTTATG
GCGGTGTGCCTGCAAGGCTCGAGGACTCCCTGATGACCAACTAACCAAATTGTA
CGTGGCAACAGATGGACGGTCCAGATTGAGCCCTCAACTCCCACCAGGCTATCT
AGGCAATGTTGTGTTCACCGCCACCCCAGTTGCCAAATCAGCTGACCTCACGAC
TCAACCATTGTCTAATGCAGCATCTTTGATCCGAACCACATTGACAAAAATGGA
TAACGACTATTTGAGATCTGCCATTGATTACCTTGAGGTGCAGCCAGATCTATC
TGCTTTAATTCGTGGTCCTAGTTACTTTGCTAGCCCGAATTTGAACATAAACACG
TGGACCCGGTTGCCAGTACATGATGCGGATTTCGGGTGGGGTCGGCCTGTTTTC
ATGGGACCAGCAGTGATATTGTATGAGGGCACCATCTATGTTCTACCAAGCCCA
AACAATGATAGGAGTATGTCATTGGCAGTCTGTTTAGATGCAGATGAACAACCA
TCGTTTGAGAAGTTCCTGTATGACTTTTAA.

In some embodiments, the polynucleotide comprises a nucleic acid sequence with at least 87%, at least 90%, at least 93%, at least 95%, or at least 99% homology or identity to SEQ ID NO: 3, or any value and range therebetween. Each possibility represents a separate embodiment of the invention. In some embodiments, the polynucleotide comprises a nucleic acid sequence with 87% to 100%, 90% to 100%, 93% to 100%, or 95% to 100% homology or identity to SEQ ID NO: 3. Each possibility represents a separate embodiment of the invention.

In some embodiments, the polynucleotide comprises or consists of the nucleic acid

(SEQ ID NO: 4)
ATGCCTTCATCATCATCATCGCCTTCTTCAACAGCTGATTCAGTTACCATAATCT
CAAAATGCACAGTCTACCCACATATGAAAAACTCAACACCAGAATCCTTGCAG
CTCTCTGTTTCTGATCTCCCAATGCTTTCATGTCAATACATACAAAAAGGTGTCT
TACTTTCTCAACCGCCACCCAATCACACCAACAATATCATTTCCCACTTAAAACT
CTCTCTCTCTAAAACCCTCTCTCACTTCCCACCTCTCGCCGGCCGTCTTTCGACC
GACTCTCACGGCCACGTCTCTATCATCTGCAACGATTCCGGCGTCGAATTCGTTC
ACTCCACCGCTAACCACCTCCACACCCACCAAATCTTACCCCTCAATTCCGACG
TTCACCCATGTTTTAAAACCTTTTTTGCTTTTGATAAAACTCTGAGTTACGCCGG
CCACCACCAACCAATCGCCGCCGTGCAAGTCACGGAGCTTGCTGATGGACTCTT
TATTGGGTGTACGGTAAATCATGCTGTCGTTGACGGGACTTCTTTTTGGAACTTT
TTTAATACTTTTGCTGAGATCACAAAAGGGTGTCAGAAAGTAACGAACTTGCCG
GATTTTAGCCGGGAAAATGTTTTCATTTCTCCGGTTGTTTTGCCTCTTCCCTCCG
GCGGCCCGTCGGCGACGTTCTCAGGTGATGAGCCGTTGAGGGAAAGGATCATT
CATTTCAGTAGAGACGCGATTCTGAAGATGAAATTCAGAGCTAATAATCCTTTA
TGGCGGCAACCACAAAATTCGGATCTGGATGATACAGAGATTTACGGGAAAGT
GTGTAACGACATTAACGGCAAAGTTAACGGGGCGTTTAAACCCAAAAGTGAAA
TTTCGTCCTTCCAGTCTTTATGTGGTCAGTTATGGCGTGCGGTTACACGCGCGCG
TAAATTCAACGACCCTATAAAAACGACGACGTTTCGAATGGCGGTGAATTGTAG
GCATAGGCTAGACCCAAAGGTCGACAAACTTTATTTCGGGAACTTGATCCAAAG
CATCCCGACCGTTGCTTCAGTTGGGGAGTTGTTATCACATGATTTGTCGTGGGC
AGCCAATGAGCTTCACCAAAATGTGGTGGCGCATGATAATGCTACCGTGCGCA
GGGGTGTTAAGGATTGGGAGAATAATCCAAAGTTGTTTCCTTTGGGGAATTTTG
ATGGTGCTATGATCACAATGGGAAGTTCTCCTAGGTTTCCAATGTATAATAACG
ATTTCGGGTGGGGCCGCCCAATGGCGGTTCGTAGTGGTAAAGCTAATAAGTTTG
ATGGAAAGATTTCGGCTTTTCCGGGACGTGATGGTGATGGTAGTGTCGATCTTG
AGGTTGTTTTAGCTCCCGAAACCATGGCATGTCTTGAACGTGACCATGAATTTA
TGCAATATGTATCTTAA.

In some embodiments, the polynucleotide comprises a nucleic acid sequence with at least 82%, at least 90%, at least 97%, or at least 99% homology or identity to SEQ ID NO: 4, or any value and range therebetween. Each possibility represents a separate embodiment of the invention. In some embodiments, the polynucleotide comprises a nucleic acid sequence with 82% to 100%, 85% to 100%, 90% to 100%, or 95% to 100% homology or identity to SEQ ID NO: 4. Each possibility represents a separate embodiment of the invention.

In some embodiments, the polynucleotide comprises or consists of the nucleic acid

(SEQ ID NO: 5)
ATGAAGTGGTTTTTCATAACCCATAAAGCAACCCAGCGTTGCCTTAATTCTAAA
CAATTTCATCTTCACGGAGGTTCGAATTTCGTTTCCGGTAATAGATGTTTTCTTG
CATCACACTCAATGGAGCGGCCAAAATTCATGTTGATACCATATTATCCCTACC
AAATTCGGTCCTTAAATTCGAGTCACCGATATAGTAGTACGTCACCCAGCGGAT
CCCCTCACAGTTTTCTGAATGGTACTAAGAATGAAAACTATACGAAGAAGGTAG
ATCTTGAAATAATTTCAAGAGAAATCATCAAACCAGCTTCTCCAACTCCACATC
ATTTAAGAAACTTCAACTTATCACTTTTGGACCAAATAGTATTTGATTGCTACAC
CCCTGTAATCCTCTTTATTCCAAATAGTAATAAGGCTACTGTTACGGATGTCATG
ATCAAAAGATTGAAACATCTCAAGGAGACTTTATCTCGAATTCTAAGTCAATTT
TATCCCTTTGCGGGAGAAGTTAAGGACAGATTGCATATCGAATGCAATGACAA
GGGAGTCAATTACATCGAGGCTCAAATCAATGAGACATTGGAAGAATTTCTATG
TCATCCAGATAACGAAAAGGCGAGGGAGCTTATGCCCGAAAGCCCTCATGTTC
AAGAATCTGCAATAGGAAACTATGCTATGGGTATTCAGATAAACATTTTCAGTT
GCGGAGGGATTGGACTTTCCATGAGCATGGCACACAAGATCATGGACTTCTACA
CATATACGATCTTCATGAAAGCATGGGCTGCAGCTGTTCGAGGTTCACCAGATA
CAATTATTTCACCAAGTTTTGTGGCTTCTGAGGTCTTTCCTAATGATCCCAGCCA
AGAAGATTCAATTCCTATCGAGTTAAAGTCTAGTAATTTGCTTAGCACAAAAAG
ATTTGAGTTTGATCCTACTGCGTTGGCTCTCCTAAAGGGACAAGTTGTCGCCAG
CGGATCACCTCCCCAACGAGGACCAAGTCGTATGGAGGCGACAACAGCCGTTA
TTTGGAAGGCCGCTGCAAAAGCTGCATCGACTGTCAGAAGATTCGATCCAAAGT
CACCTCATGCGCTGGCGTTACCAGTAAATATACGTAAAAGGGCATCACCTGCTC
TCCCAGACAATTCCATAGGAAACATAGTTATGCGAGGTATAGCAATTTGTTTTC
CTGAGAGCCAACCGGACTTGCCAACTCTTATGGGTAAAGTGAGAGAATCAATA
GCGAAACTTAACTCAGATTACATTGAGTCCCTGAAAGGTGAAAAGGGGCATGA
GACAGTTAATAAGATGTTGAAGGAGTTGAAGCTTCGGACGAATATGACAAAGG
TAGGAGGGAAATTCGTTGCTAGTTGCATATTTAATAGTGGAATATATGAGTTGG
ATTTCGGGTGGGGAAAACCGATATGGTTCTATGTTGTGAATCCAGGAAGCGATA
GTTGTGTGGTTTTGACTGATACGCTGAAGGGTGGTGGTGTTGAAGCCACAATTA
CACTACCACCAGATGAAATGGAGATATTCGAACGTGATCATGAGCTTCTATCCT
ATACTACCATCAACCCTAGTCCACTGCGATTTCTTGACCATTGA.

In some embodiments, the polynucleotide comprises a nucleic acid sequence with at least 74%, at least 80%, at least 85%, or at least 95% homology or identity to SEQ ID NO: 5, or any value and range therebetween. Each possibility represents a separate embodiment of the invention. In some embodiments, the polynucleotide comprises a nucleic acid sequence with 74% to 100%, 80% to 100%, 87% to 100%, or 95% to 100% homology or identity to SEQ ID NO: 5. Each possibility represents a separate embodiment of the invention.

In some embodiments, the polynucleotide comprises or consists of the nucleic acid sequence:

(SEQ ID NO: 6)
ATGGAGGTGCCTGACCAATTCCACCTAAACATTCTTGAACAATGCCACGTTTCA
CCATCACCAAATTCCATCATACCTTCATTTTCACTACCCTTAACATTCTTAGACA
TCCCATGGCTTTTTTACCCTTCAAATCAAACCCTTTTTTTCTTCCCAGAACCACC
ACCCAAAACCACCATCATCACCACCCTTAAACAATCACTCTCTCTTACCCTCCA
CCACTTCCACCCTCTCGCCGGAAACCTCTCACTTCCATCACCTCCGGCGGAACC
CCACATTGTTTACACCAAAAATGACTCAATTGCACTCACAATTGCTCAAACAAA
CACCAACATCCACCATCTTTCTTGCAATCACCCAAGAAGTGTAAAAAATCTTTA
CTCTCTTTTACCCAAACTCCCATCTCCATCCATGTCACGTGAAACTCACGTGGGC
CTTGTTATCCCCCTTCTTACCATCCAAATTACGGTTTTTGCTGATTTGGGGTATTC
GATCGGAGTCACTATGCAACATGCAGCAGTTGATGAACGGACATTTGATCAGTT
TATGAAATGTTGGGCGTCTGTTTGTACATCTTTGTTGAAAAATGACTCACTTTTT
ACATTCAAGTCTACACCTTGGTACGATAGGAGCGTAATTATCGACCCCAAATCG
CTGAAAACAACGTTTTTAAAGCAATGGTGGAACCGATCTAATTCTCTCAATGAG
TCACATGATCAAGAAAATGATGATCATGATCTTGTTCTAGCAACTTTTGTTTTGA
GTTCATTAGATATTAACATGATCAAGAATCATATTCTTGCAAAATGCAAGATGA
TAAATGAGGATCCACCACTACATTTATCTCCTTATGTTAGTGCATGTGCTTATTT
ATGGAAATGTTTAATCAAAATTCAAGAAACCCATGATTCTATTAAGGGTGGTCC
TCTCTATTTAGGGTTTAATGCCGGTGGGATTACTCGATTAGGGTACGACATACC
TTCAACTTATTTTGGGAATTGTATAGCTTTTGGGAGATGCAAGGCATTTGAGAG
TGAATTATTGGGTGATAATGGTATTGTTTTCGCGGCAAAATCGATTGGAAAAGA
GATCAAGAGGCTTGATAAGGATGTTTTAGGAGGTGCTAATAAGTGGATTAGTG
ATTGGGATGAATTAACCATTAGGCTTCTTGGTTCACCAAAAGTTGATTCATATG
GTATGGATTTTGGATGGGGTAAAGTTGAGAAGGTTGAAAAAATATCAAGTATTT
CAAATCACGGTAGGGTTAATGTAATTTCTTTGAGTGGATGTAAGGATTTTAAAG
GTGGAATAGAGATAGGGGTTGTTCTTTCTGTGGCTAAAATGAATGTTTTCACTT
CCCTCTTTCATGGAGGTTTAATGGAGTTTGCATATTGA.

In some embodiments, the polynucleotide comprises a nucleic acid sequence with at least 79%, at least 87%, at least 93%, at least 97%, or at least 99% homology or identity to SEQ ID NO: 6, or any value and range therebetween. Each possibility represents a separate embodiment of the invention. In some embodiments, the polynucleotide comprises a nucleic acid sequence with 79% to 100%, 85% to 100%, 90% to 100%, or 95% to 100% homology or identity to SEQ ID NO: 6. Each possibility represents a separate embodiment of the invention.

In some embodiments, the polynucleotide comprises or consists of the nucleic acid sequence:

(SEQ ID NO: 7)
ATGAAAAATAAGAACCCGACTAGTGTGATCAGAGAGGCTTTAGCTAAGGTATT
GGTGTTTTATTATCCGTTTGCTGGCCGGCTCAAGGAAGGGCCGGCCAGGAAACT
GATGGTGGATTGTTCTGGTGAAGGTGTGTTGTTTATTGAGGCAGAAGCTGATGT
CACGTTGAAACAATTTGGTGACGCACTTCAACCGCCATTTCCTTGTTTAGAAGA
GCTTCTTTACGATGTTCCTGGATCTACTGGTATTCTAGATACACCATTATTGCTG
ATTCAGGTGACACGATTGTTATGTGGAGGTTTTATCTTTGCTCTACGACTCAACC
ACACCATGAGCGACGCAGCAGGTCTCGTTCAATTCATGACAGGGCTTGGTGAA
ATGGCACAAGGTGCATCAAGGCCATCAACGTTGCCTGTATGGCAAAGGGAGTT
GCTTTTTGCAAGGGACCCACCACGCGTGACTTGTACTCATCACGAGTATACTGA
AGTGGAAGACACCAATGGTACAATCATTCCGCTAGATGACATGGCACATAAAT
CATTTTTCTTTGGACCTTCTGAGATATCAGCGTTGCGAAGGTTCGTTCCATCATA
CCTAAAAAAGTGTTCTACTTTTGAGGTCTTAACCGCTTGCCTATGGCGTTGTCGT
ACAATTGCACTCCAGCCAGATCCCGAAGAAGAGATGCGCATGATATGCATTGTT
AATGCGCGTGGAAAGTTTAATCCTCCCCTATTACCCAAAGGATATTATGGAAAT
GGTTTCGCTATACCAGTGGCCATTTCAACAGCTGGAGACCTATCTAGCAAACCA
TTAGGTCACGCATTGGAACTTGTAATGAAAGCCAAATCCAATGTCACTGAGGAG
TATATGAGATCAGTAGCCGACTTAATGGTAATCAAGGGACGACCCCACTATACG
GTTGTCCGAAGCTACCTTGTATCGGATGTGACTCACGCTGGATTTGATGTTGTTG
ATTTCGGGTGGGGGAAAGCGTCCTATGGAGGACCTGCAAAAGGGGGAGTAGGT
GCTATTCCCGGAGTTGTTACTTTCTTTATACCTTTTACAAACCATAAAGGCGAGT
CTGGAATTGTGCTACCTATATGTTTGCCGAGTGCAGCCATGGATAAGTTTGTTG
AAGAGTTAAATAAGATGTTGGTCCCAGACAACAACGAACAAGTACTCCGAGAA
CACAAGTTACTAGTTCTCGCTAGATTGTAA.

In some embodiments, the polynucleotide comprises a nucleic acid sequence with at least 82, at least 85%, at least 95%, or at least 99% homology or identity to SEQ ID NO: 7, or any value and range therebetween. Each possibility represents a separate embodiment of the invention. In some embodiments, the polynucleotide comprises a nucleic acid sequence with 82% to 100%, 85% to 100%, 90% to 100%, or 95% to 100% homology or identity to SEQ ID NO: 7. Each possibility represents a separate embodiment of the invention.

In some embodiments, the polynucleotide comprises or consists of the nucleic acid sequence:

(SEQ ID NO: 8)
ATGGCACAAATCGACACTCCATTGACATTCAAAGTCCGGAGACATGCACCGGA
GCTGATCGCTCCAGCGAAACCTACGCCACGAGAACTAAAACCTCTATCCGACAT
TGATGATCAAGAAGGCCTTAGGTTTCATATCCCAGTGATTCAATTCTATCGTAG
CGATCCAAAGATGAAAAATAAGAACCCGGCTAGTGTGATCAGAGAGGCTTTAG
CTAAGGTGTTGGTGTTTTACTATCCGTTTGCTGGCCGGCTCAAGGAAGGGCCTG
CCAGGAAACTGATGGTAGATTGCTCTGGTGAAGGTGTGTTGTTTATTGAGGCGG
AAGCTGATGTCACGTTGAAACAATTTGGTGACGCCCTTCAACCGCCGTTTCCTT
GTTTGGAAGAGCTTCTTTACGATGTTCCTGGATCTACTGGCGTTCTAGATACACC
GTTATTGCTGATTCAGGTGACACGATTGTTATGTGGAGGTTTTATCTTTGCTCTA
CGACTCAATCACACCATGAGCGACGCACCAGGTCTCGTTCAATTCATGACAGGG
CTCGGTGAAATGGCACAAGGTGCATCAAGGCCATCTACGTTGCCTGTATGGCAA
AGGGAGTTGCTTTTAGCAAGGGACCCACCACGCGTGACATGTACTCATCACGAG
TATACTGAAGTGGAAGACACCAAGGGTACAATCATTCCGCTAGATGACATGGC
ACATAAATCATTTTTCTTTGGACCTTCTGAGATATCAGCATTGCGAAGGTTCGTT
CCATCATACCTAAAAAAGTGTTCTACTTTTGAGGTCTTAACCGCTTGCCTATGGC
GTTGTCGTACAATTGCACTCCAGCCAGATCCCGAAGAAGAGATGCGCATAATAT
GCATTGTTAATGCGCGCGGAAAGTTTAATCCACCCCTTCCTAAAGGTTATTATG
GAAATGGTTTTGCTTTCCCAGTGGCCATTTCAACAGCTGGAGATCTATCCAGCA
AACCATTAGGTCATGCATTGGAACTTGTAATGAAAGCCAAATCCGATGTCACTG
AGGAGTATATGAGATCAATAGCCGACTTAATGGTAATCAAGGGACGTCCCCAC
TTTACGGTTGTCAGAAGCTACCTTGTCTCGGATGTGACTCACGCTGGATTTGATG
TTGTTGATTTCGGGTGGGGGAAAGCGGCCTATGGAGGACCCGCTAAAGGGGGA
GTAGGTGCTATCCCAGGTGTTGCTAGTTTCTATATACCTTTTACAAACCATAAAG
GCGAGTCTGGAATTGTGCTACCTATATGTTTGCCGAGTGCGGCCATGGATAAGT
TTGTTGAAGAGTTAAATAAGATGTTGGTCCCAGACAACAACGAACAAGTACTCC
GAGAACACAAGTTACTAGTTCTTGCTAGATTGTAA.

In some embodiments, the polynucleotide comprises a nucleic acid sequence with at least 83%, at least 85%, at least 89%, at least 92%, at least 95%, at least 97%, or at least 99% homology or identity to SEQ ID NO: 8, or any value and range therebetween. Each possibility represents a separate embodiment of the invention. In some embodiments, the polynucleotide comprises a nucleic acid sequence with 83% to 100%, 88% to 100%, 92% to 100%, or 95% to 100% homology or identity to SEQ ID NO: 8. Each possibility represents a separate embodiment of the invention.

In some embodiments, the polynucleotide comprises or consists of the nucleic acid sequence:

(SEQ ID NO: 9)
ATGGAAATACAAGTAATAAACTACTCATCAAAGCTAGTAAAACCCTTGACACC
AACACCCACCGCAAATCGTTACTATAACATTTCTTTCACCGATGAGCTCGTCCC
AACCATTTACGTCCCACTCATTCTCTACTACGCAACACCGAAAAACCCAAATGG
TGATCACTTTGAAAACATTTGTGACCGTCTGGAGGAGTCGTTATCGAAAACGTT
AAGTGATTTTTACCCACTGGCCGCGAGATTCATTCGTAAACTCTCCTTAATTGAT
TGTAACGATCAAGGGGTTTTGTTTGTCCTAGGCAATGTAAATATCCGACTTTCG
GATGTTACAGGCCTAGGACTGACGTTTAAAACCAGTGTTTTAAATGATTTTCTC
CCGTGTGAGATTGGAGGAGCGGATGAAGTCGATGATCCTATGCTTTGTGTCAAA
GTCACCACTTTTGAGTGTGGTGGTTTTGCAATTGGTATGTGTTTTTCGCATAGGC
TTTCGGATATGGGTACCATGTGTAACTTTATTAACAATTGGGCTGCTAGAACTA
TTGGTGAATATGATAATGAAAAACATACTCCTATTTTTAATTCGCCGTTGTACTT
CCCGCAACGAGGATTACCTGAACTTGACCTAAAAGTACCTAGGTCAAGTATTGG
TGTGAAAAATGCAGCACGCATGTTTCACTTTAATGGGAAGGCAATATCATCCAT
GAGAGAAGTTTTTGGAGTTGATGAAAATGGGTCTCGTAGACTCTCAAAGGTTCA
ACTTGTTGTAGCCTTGTTGTGGAAGGCCTTTGTTCGCATAGATGATGTGAACGA
TGGCCAATCTAAGGCGTCTTTTCTGATCCAACCAGTTGGGTTGAGGGACAAAGT
TGTCCCTCCATTACCATCAAACTCATTTGGGAATTTTTGGGGTCTAGCGACTTCC
CAACTTGGTCCTGGTGAGGGTCACAAAATCGGTTTCCAAGAATATTTTTACATT
TTGCGTGAATCTATTAAGAAAAGAGCTAGGGATTGCGCTAAAATATTGACACAC
GGTGAAGAAGGATATGGGGTTGTAATCGATCCATATCTTGAGTCGAATCAAAA
GATAGCTGATAATGGTACAAACTTTTACTTGTTCACTTGTTGGTGCAAGTTTTCG
TTCTACGAAGCTGATTTTGGTTGTGGTAAGCCGATTTGGGCTAGCACCGGAAAG
TTTCCGGTTCAAAATTTGGTGATCATGATGGATGATAATGAGGGTGATGGTGTA
GAAGCGTGGGTTCATTTAGACGATAAACGCATGAATGAGTTAGAACAAGATCC
TGATGTTAAACTCTACGCATGCAATTTAGCTTAA.

In some embodiments, the polynucleotide comprises a nucleic acid sequence with at least 77, at least 85%, at least 95%, or at least 99% homology or identity to SEQ ID NO: 9, or any value and range therebetween. Each possibility represents a separate embodiment of the invention. In some embodiments, the polynucleotide comprises a nucleic acid sequence with 77% to 100%, 82% to 100%, 87% to 100%, or 95% to 100% homology or identity to SEQ ID NO: 9. Each possibility represents a separate embodiment of the invention.

In some embodiments, the polynucleotide comprises or consists of the nucleic acid sequence:

(SEQ ID NO: 10)
ATGAAATTAGCAGTGAAGGAATCAGTGATAGTAAAACCATCCAAAACGACACC
GTGTCAGCAAATATGGACATCAAATCTTGATTTAGTGGTGGGTCGGATCCATAT
ATTAACCGTTTACCTTTACAGACCAAATGGGTCTTCAAATTTCTTTGATTCCATG
GTTTTAAAGAAGGCTCTAGCCGACGTTTTAGTTTCTTTTTTTCCGGTGGCCGGAC
GGTTGGATAAAGACGGTGACGGCAGAGTTGTAATAGATTGTAACGGTGAGGGT
GTTTTGTTTGTGGAAGCTGAAGCTGATTGTTGCATTGATGATTTTGGTGAGATTA
CTCCGTCGCCGGAGTTACGACGGTTGGTGCCGACGGTGGATTATTCCGGTGATA
TGTCTTCTTATCCGTTATTTATTACGCAGGTTACACGGTTCAAGTGTGGGGGAGT
TTCGTTAGGCTGTGGACTACACCATACGTTATCGGATGGACTCTCAGCACTTCA
CTTCATCAACACATGGTCTGATGTAGCTAGAGGCCTATCGGTGGCAATCCCACC
GTTCATTGACCGCTCCCTTCTTCGAGCTCGTGATCCACCATCCCCTGTGTTTGAC
CACATCGAATACCACCCACCACCGTCACTGATCACTCCGTTGCAAAACCAAAAG
AACGCGTCACATTCGAGGTCTGCTTCAACTTTAATCCTACGGCTCACACTCCATC
AAATAAACAATCTTAAATCAAAGGCTAAAGGCGATGGGAGCATGTACCATAGC
ACGTACGAGATCCTAGCTGCTCATCTATGGCGATGTGCGTGCAAAGCACGTGGA
CTAGCAAACGATCAACCAACCAAATTGTATGTGGCCACCGATGGACGGTCAAG
ATTGATTCCTCCACTCCCTCCGGGCTACCTTGGGAATGTCGTTTTCACGGCTACT
CCTGTCGCTAAATCGGGAGATTTCGAATCTGAATCCTTGGCAGAGACAGCAAGG
AGGATTCGCAGTGAGTTGGGTAAAATGAACGATGAGTATCTTAGATCAGCTATT
GACTACTTAGAGTCGGTATCTGATATTTCGACCCTTGTTAGAGGGCCGACTTAC
TTTGCGAGTCCAAATCTGAATGTAAACAGTTGGACTCGGTTACCAATATACGAA
TCTGACTTCGGTTGGGGTCGACCTATTTTCATGGGACCCGCAAGTATACTTTACG
AGGGTACGATTTACATCATACCGAGCCCTAGTGGTGATCGGAGTGTGTCTCTGG
CCGTGTGCTTGGACCCTGATCACATGGCTTTGTTTAAAGAATGCTTGTACGTTTT
TTAG.

In some embodiments, the polynucleotide comprises a nucleic acid sequence with at least 84, at least 89%, at least 95%, or at least 99% homology or identity to SEQ ID NO: 10, or any value and range therebetween. Each possibility represents a separate embodiment of the invention. In some embodiments, the polynucleotide comprises a nucleic acid sequence with 84% to 100%, 88% to 100%, 93% to 100%, or 97% to 100% homology or identity to SEQ ID NO: 10. Each possibility represents a separate embodiment of the invention.

In some embodiments, the polynucleotide comprises or consists of the nucleic acid sequence:

(SEQ ID NO: 11)
ATGAAGCTAGCAGTGAAGGAATCAGTGATAGTAAAACCATCCAAAACGACACC
GTGTCAGCAAATACGGACATCAAATCTTGATTTAGTGGCGGGTCGGATCCATAT
ATTAGTCGTTTTCTTTTACAGACCAAATGGGTCTTCGAATTTCTTTGATTCCTTG
GTTTTAAAGAAGGCTCTCGCCGACGTTTTAGTTCCTTTTTTTCCGGTGGCCGGAC
GGTTCAGTGAAGACGGTGACGGCAGAGTTGTAATTGATTGTAACGGTGAGGGT
GTTTTGTTTGTGGAATCTGAAGCTGATTGTTGCATTGATGATTTTGGTGAGATTA
CTCTGTCGCCGGAGTTACAACAGTTGGTGCCGACGGTGGATTATTCCGGTGATA
TGTCTTCTTATCCGTTATTTATTGCGCAGGTCACACGGTTCAAGTGTGGGGGAGT
TTCGTTAGGTTGGGGACTACACCATACATTATTGGATGGACTCTCAGCACTTCA
CTTCGTCAACACATGGGGTGATGTAGCTAGAGGCCTATCGGTGGCAATCCAACC
GTTCATTGACCGCTCCCTTCTTCGAGCTCGTGATCCACCGACCCCTGTGTTTGAC
CACATCGAATACCACCCACCACCGTCACTGATCACTCCATTGCAAAACCAAAAG
AACGCATCACATTCGAGGTCTGCTTCAACTTTAATCCTACAGCTCACACCCGAT
CAAATAAAGAATCTTAAATCAAAGGCTAAAGGCGATGGGAGCATGTACCATAG
CACATACGAGATCCTAGCTGCTCATCTATGGCGATGTGCGTGCAAAGCGCGTGG
ACTAGCAAACGATCAACCAACCAAATTGTATGTGGCCGCCAATGGACGGTCAA
GATTGATTCCTCCACTCCCTCCGGGCTACCTTGGGAATGTCGTTTTCAACGCTAC
TCATGTCGCTAAATCGGGGGATTTTGAATCTGAATCCTTGGCAGAGACTGCAAG
GAGGATTCACTGTGAGTTGGGTAAAATGAACGATGAGTATTTTAGATCAGCTAT
CGACTACTTAGAGTCGGTAGATGATATTTCAACCCTTGTCAAAGGGCCGACTTA
CTTTGCGAGTCCAAATCTGAATGTATACAGTTGGATTGGGATACCAATATATGC
ATGTGACTTCGGATGGGGTCAACCTATTTTCATGAGACCCGCAAGTTTCCTTTAC
GATGGTTCCATTTACATCATACCGAGCCCTAGTGGTGATCGGAGTGTGTTGTTG
GCCGTGTGCTTGGACCCTGATCACATGGATTTGTTTAAAGAATGCTTGTACGCTT
TTTAG.

In some embodiments, the polynucleotide comprises a nucleic acid sequence with at least 82, at least 85%, at least 95%, or at least 99% homology or identity to SEQ ID NO: 11, or any value and range therebetween. Each possibility represents a separate embodiment of the invention. In some embodiments, the polynucleotide comprises a nucleic acid sequence with 82% to 100%, 85% to 100%, 90% to 100%, or 95% to 100% homology or identity to SEQ ID NO: 11. Each possibility represents a separate embodiment of the invention.

In some embodiments, the polynucleotide comprises or consists of the nucleic acid sequence:

(SEQ ID NO: 12)
ATGGTGATGATTAGCAAGCTTTTACGATTAGGTAGAAGAAAACTTCACACAATT
GTATCAAGAGATACCATTAGACCTTCTTCTCCAACTCCCTCTCATTCCAAAACAT
ATAATCTCTCCTTGCTCGATCAAATAGCTGTAAATTCATACGTGCCGATTGTTGC
TTTTTACCCAAGCTCAAATGTTTGTCGAAGTTCCGATGATAAGACGCTGGAGTT
GAAGAACTCATTATCGAAAATATTAACTCATTACTATCCGTTTGCCGGTAGAAT
GAAGAAGAATCGCCCTACCGTCGTTGATTGCAATGATGAAGGGGTTGAGTTCGT
TGAAGCACGTAATACCAACTCGTTATCAGATTTCCTCCAACAATCGGAGCACGA
AGATCTAGATCAACTCTTTCCAGATGATTGTGTATGGTTCAAACAAAACCTTAA
AGGTTCTATTAATGACGCAAATAATAGTAGCGTATGTCCATTGAGCATTCAAGT
CAACCATTTCGCGTGTGGAGGTGTAGCAGTTGCAACTTCGTTACGCCACAAGAT
TGGAGACGGAAGCAGTGCGTTAAATTTCATTAAACACTGGGCTGCAGTTACGTC
ACACTCTCGAGCAGGGAATCATCAAATTGATGCGACATCACCCATCATTAATCC
CCATTTCATTTCTTACCCAACTAGAACTTTTAAATTGCCAGATAGGTCACCATAC
ATACCACCTAGTGATGTTGTGTCAAAAAGTTTTGTTTTCCCCAACACAAATATA
AAGGACCTCCAAGCCAAGGTGGTAACCATGACCATGGGCTCTAGACAACCTAT
CGTGAACCCTACCCGAGCTGATGTCGTATCATGGCTTCTACATAAGTGTGTAGT
AGCAGCAGCTACCAAAAGGATATCGGGAAATTTTAAAGAAAGTTGCGTGATCT
CGCCATTAAATCTGAGAAACAAGTTAGAAGAGCCATTGCCTGAAACAAGCATA
GGAAATATTTTCTATCTGATAACCTTTCCAATAAGCAATAATCATGGCGATCTC
ATGCCCGATGACTTCATTAGCCAACTCAGGCTAGGAATACGTAAGTTTCAAAAT
ATACGAAATTTGGAAACTGCATTACGAACCGTTGAAGAGATGATATCTGAAACT
TTTATCTTGGGTACGGCAGAAAGCATGGATACTAGTTATGTATATTCGAGCATC
CGTGGGTTTCCGATGTATGATATTGATTTTGGGTGGGGGAAGCCCGTAAAAGTA
ACCGTTGGGGGAGCCCTTAAGAACTTAAGTATTCTGATGGACACTCCTGATGTC
AATGGCATCGAAGCACTAGTGTCTTTGGATAAACAAGACATGAAGATACTTCTA
AACGACCCTGAGTTGTTGGCCTTTTGCTTGTAA.

In some embodiments, the polynucleotide comprises a nucleic acid sequence with at least 72, at least 80%, at least 85%, at least 87%, at least 93%, or at least 99% homology or identity to SEQ ID NO: 12, or any value and range therebetween. Each possibility represents a separate embodiment of the invention. In some embodiments, the polynucleotide comprises a nucleic acid sequence with 72% to 100%, 79% to 100%, 86% to 100%, or 91% to 100% homology or identity to SEQ ID NO: 12. Each possibility represents a separate embodiment of the invention.

In some embodiments, the polynucleotide comprises or consists of the nucleic acid sequence:

(SEQ ID NO: 13)
ATGAGTACTAGTGACAAAATGAAGATAACAATAAGAGAATCATCAATGATAAA
ACCATCCAAACCGACGCCGGATCAACGGATATGGAACTCAAATCTTGATTTGGT
AGTGGGTCGGATCCATATCTTGACCCTTTACTTTTTTAGGCCAAATGGGTCTTCG
GATTTCTTTGATTCTGAGGTTTTAAAGCAATCACTTGCCGACGTTCTTGTTTCTTT
TTTTCCGATGGCCGGACGATTGGGATTAGACGGCGATGGCAGAGTTGAAATTAA
TTGCAACGGTGAAGGTGTTTTGTTTGTTGAAGCTGAAGCGGATTGTAGTATTGA
TGATTTTGGTGAGATTACTCCGTCGCCGGAGCTACGGCGGTTGGCGCCAACAGT
GGATTATTCCGGCGATATCTCATCTTATCCACTCGTTATTACCCAGGTAACACAT
TTCAAATGTGGTGGAGTTTCTCTTGGGTGTGGACTACACCATACATTATCCGAT
GGACTTTCATCTCTTCACTTCATCAACACATGGTCCGATGTTACCCGAGGCTTAC
CCGTTGCGATCCCGCCATTCGTAGATCGTACGGTTCTTCGTGCTAGGGACCCGC
CAACCGTGGTCTTTGATCACGTGGAATACCACACTCCTCCTTCCATGACCTCAA
GTTTGGACAAAGACAAACCTCAATCCGAAGATGTTCATGTTTCCACTTCCATGC
TACGGCTCACACTCGATCAAATAAATGCACTAAAAGCAAAAGGCAAAGGTGAC
GGAATTGTGTACCATAGCACATATGAAATCCTAGCTGCTCATTTATGGCGATGT
GCGTGTAAAGCACGTGGGCTCCTGAATGATCAAATGACTAAATTGTATGTAGCT
ACCGATGGACGGTCCAGATTGATTCCCCCACTCCCACCGGGGTACTTAGGCAAT
GTGGTCTTCACCGCCACACCAATTGCCAAATCCGGCGAGCTCCAACAGGAACCA
CTAGCTACCACTGCAAGAAAAATTCATACAGAGTTGGCCAAAATGGATGACAA
GTACCTCAGGTCGGCCCTCGACTACTTAGAGTCACAACAGGACTTGTCAGCACT
AATTCGAGGGCCAGCCTATTTTGCGTGCCCTAACCTCAACATCAATAGTTGGAC
TCGCCTTCCAATATATGATGCGGACTTTGGGTGGGGTCGGCCCATATTTATGGG
ACCCGCCAGCATACTTTACGAGGGCACGATTTACATTATTCCGAGCCCTAGTGG
TGACCGAAGTGTGTCGTTGGCTGTGTGCTTAGACCCCTCTCATATGCCTCTCTTC
CAAAAGTACTTGTATGAACTTTAA.

In some embodiments, the polynucleotide comprises a nucleic acid sequence with at least 79, at least 85%, at least 95%, or at least 99% homology or identity to SEQ ID NO: 13, or any value and range therebetween. Each possibility represents a separate embodiment of the invention. In some embodiments, the polynucleotide comprises a nucleic acid sequence with 79% to 100%, 85% to 100%, 90% to 100%, or 95% to 100% homology or identity to SEQ ID NO: 13. Each possibility represents a separate embodiment of the invention.

In some embodiments, the polynucleotide comprises or consists of the nucleic acid sequence:

(SEQ ID NO: 14)
ATGGTGAATGTTGAGATCATTTCTAATGAATACATAAAACCATCCTCCCCAACA
CCACCACATCTTAAAATATACAATCTTTCCATCTTAGATCAACTCATTCCTGCCC
CCTATGCACCTATCATACTATATTATCCGAATCAAGATCACATTAACGATTTTGA
GGTTCACGAACGGTTGAAACTACTAAAAGATTCGTTATCGAAAACGCTAACTCG
TTTTTACCCATTAGCCGGAACCATCAAAGGCGATCTTTCCATTGATTGTAACGAT
ATTGGTGCTTACTTTGCAGTAGCTCATGTAAATACTCGCCTTGATGTGTTCCTGA
ACCATCCTGATCTTGACCTAATAAACTGTTTTCTTCCACGTGGGCCTTACTTGAA
TGGTTCTAGTGAAGGAAGTTGTGTGAGTAATGTTCAAGTGAACATTTTTGAGTG
TTGTGGGATTGCAATTAGTTTATGCATTTCTCACAAGATTCTTGATGGTGCTGCG
TTGAGTACTTTTCTTAAAGCATGGGCAGGGACAAGTTACGGGTCGAAAGAAGT
AGTGTATCCAAACATGAGTGCACCATCTTTATTTCCTGCTAAAGATTTGTGGCTT
AAAGATTCATCAATGGTCATGTTTGGGTCTTTGTTTAAGATGGGTAAGTGTAGT
ACTAAAAGATTTGTTTTTGATTCATCAAAATTATCCTTCCTCAAAGCTAAGGCAT
CGCTAAATGGGCTAAAAGACCCAACCCGCGTAGAGGTGGTGTCTGCTTTACTAT
GGAAGTGTATCATGGCTGCATCTGAAGAAAACACTGGTTCTTGGAAGCCATCTC
TGTTAAGCCATGTAGTTAACCTTCGCAAAAGGTTGGTTTCAACTTTATCAGAAG
ACTCAATTGGGAACTTAATTTGGTTAGCAAGCGCAGAATGTAGAACCAACGCTC
AATCCCGATTGAGTGATCTTGTTGAAAAGGTACGTGATAGTGTGTCGAAAATCA
ATAGTGAGTTTGTGAAGAAAATACAAGGCGATAAAGGGACAAAAGTGATGGAA
GAGTCTCTCAAGAGTATGAAAGATTGTGCGGATTATATCGGGTTTACGAGTTGG
TGTAAGATGGGGTTTTACGATGTGGATTTTGGTTGGGGAAAGCCTGTATGGGTT
TGTGGTAGCGTTTGTGAAGGTAGCCCGGTGTTCATGAATTTTGTCATATTAATG
GACACAAAATATGGTGATGGAATAGAAGCATGGGTGAGCTTGGATGAACACGA
AATGCATATCTTAAAGCATAATCCCGAGCTCTTGGAATATGCATCAATCGATCC
AAGTCCTCTGCAAATGAATAAGTGA.

In some embodiments, the polynucleotide comprises a nucleic acid sequence with at least 82, at least 85%, at least 95%, or at least 99% homology or identity to SEQ ID NO: 14, or any value and range therebetween. Each possibility represents a separate embodiment of the invention. In some embodiments, the polynucleotide comprises a nucleic acid sequence with 82% to 100%, 88% to 100%, 93% to 100%, or 97% to 100% homology or identity to SEQ ID NO: 14. Each possibility represents a separate embodiment of the invention.

In some embodiments, the polynucleotide comprises or consists of the nucleic acid sequence:

(SEQ ID NO: 15)
ATGGGAACTATTTATCAATCTCCCATGATCAAATCTTCTACTCCCAAAATAATTG
AAGACCTCAAAGTTATCATCCATGACACATTCACAATCTTCCCACCTCACGAAA
CCGAAAAGCGGTCCATGTTCTTATCGAACATTGACCAAGTTCTTACTTTCAACG
TTGAAACGGTCCATTTTTTTGCAGCCAACCCTGACTTTCCGCCACAAGTAGTGG
CGGAAAAGCTCAAGTTGGCTCTAAGTAAGGCGCTGGTGCCATATGATTTTTTGG
CAGGGAGGTTGAAGTTGAACCATGAGTCGCAACGGTTTGAGTTTGATTGTAATG
GTGCTGGGGCTCGGTTCGTGGTGGGTTCGAGTGAGTTTGAGTTGGGTGAGATTG
GTGACTTGGTGTATCCAAACCCTGGGTTTAGACAATTGGTTCAAAAGAGTTATG
ATAACTTGGAGTTACATGAAAAGCCACTATGCATTTTACAGCTGACATCCTTCA
AGTGTGGAGGATTTGCACTTGGTGTAGCAACAAATCATGCCACTTTTGATGGCT
TAAGTTTCAAAACATTTCTTCAAAATCTTGGTTCTTTGGCTGCTGATCAACCACT
TGCCGTCGATCCCTGCAACGATCGCCACCTATTGGCAGCACGATCACCACCAAA
AGTCCAATTTGACCACCCTGAACTCCTCAAAATCCCAACAGGAACAGACATCCC
AAACCCAACAGTCTTTGACTGCCCAGAAAGTCAACTTGACTTCAAGATTTTCAA
CTTGACCTCAGATGACATAGCCCACTTAAAAACGAAAGCCAAAGATGGGCCTG
GGTCAACCAATGCAAAAATCACTGGATTCAATGTGGTTGCAGCCCATGTATGGC
GGTGCAAAGCGTTGTCCTCAGGGTCAGAATATGACCCCGAGAGAGTGTCAACC
GTGTTATATGCTGTTGACATTCGGTCAAGATTGAACTTACCATTATCATTAGCTG
GCAATGCAGTTCTTAGTGCATACGCCTCGGCCAAATGCAAAGAGATTGAAGAA
GGCCCGTTGTCAAGACTAGTGGAAATGGTGACCGAAGGTACTAACAGAATGAC
TGGTGAGTATGCAAGATCGGTGATCGATTGGGGAGAGGTGAATAAAGGGTTTC
CAAATGGGGAGTTTCTGATATCGTCATGGTGGCGATTGGGGTTTGCTGACGTGG
AATATCCGTGGGGTAAACCTAGGTATAGTTGTCCCGTGGTTTATCATAGGAAAG
ATATAATATTACTCTTTCCGGATATTGTTGGTGCCGATAACAACAATGAAGTGA
ATGTGTTGGTGGCTTTGCCTGGCAAAGAAATGGAGAAATTTGAGACTTTATTTC
ATAAGTTTTTGGCATGA.

In some embodiments, the polynucleotide comprises a nucleic acid sequence with at least 87, at least 91%, at least 95%, or at least 99% homology or identity to SEQ ID NO: 15, or any value and range therebetween. Each possibility represents a separate embodiment of the invention. In some embodiments, the polynucleotide comprises a nucleic acid sequence with 87% to 100%, 90% to 100%, 94% to 100%, or 97% to 100% homology or identity to SEQ ID NO: 15. Each possibility represents a separate embodiment of the invention.

In some embodiments, the polynucleotide comprises a nucleic acid sequence set forth in SEQ ID Nos: 4 or 15.

In some embodiments, the polynucleotide of the invention comprises 1,000 to 1,800 nucleotides. In some embodiments, the polynucleotide of the invention is 1,100 to 1,550 nucleotides long.

In some embodiments, 1,000 to 1,800 nucleotides comprises: at least 1,050 nucleotides, at least 1,150 nucleotides, at least 1,200 nucleotides, at least 1,300 nucleotides, at least 1,400 nucleotides, at least 1,500 nucleotides, at least 1,600 nucleotides, at least 1,700 nucleotides, at least 1,750 nucleotides, or at least 1,790 nucleotides, or any value and range therebetween. Each possibility represents a separate embodiment of the invention. In some embodiments, 1,000 to 1,800 nucleotides comprises: 1,050 to 1,790 nucleotides, 1,100 to 1,750 nucleotides, 1,200 to 1,650 nucleotides, or 1,250 to 1,600 nucleotides. Each possibility represents a separate embodiment of the invention.

In some embodiments, the polynucleotide comprises a plurality of polynucleotides. In some embodiments, the polynucleotide comprises a plurality of types of polynucleotides. As used herein, the term “plurality” comprises any integer equal to or greater than 2. In some embodiments, the polynucleotide comprises at least 2, at least 3, at least 4, at least 5, at least 6, at least 7, at least 8, at least 9, at least 10, at least 11, at least 12, or 13 different nucleic acid sequences, or any value and range therebetween, wherein each of the different nucleic acid sequences is selected from SEQ ID Nos.: 1-15. Each possibility represents a separate embodiment of the invention. In some embodiments, the polynucleotide comprises 2-13, 2-10, 2-8, 2-15, 3-7, 3-9, 3-12, 5-10, 5-14, or 3-15 different nucleic acid sequences, wherein each of the different nucleic acid sequences is selected from SEQ ID Nos.: 1-15.

In some embodiments, the polynucleotide is or comprises a plurality of polynucleotide molecules, wherein each of the plurality of the polynucleotide molecules comprises a different nucleic acid sequence, and wherein the different nucleic acid sequences are selected from SEQ ID Nos.: 1-15.

In some embodiments, the polynucleotide encodes a protein characterized by being capable of acting on an acyl group. In some embodiments, the polynucleotide encodes a protein characterized by catalytic activity of transferring an acyl group from a donor molecule to an acceptor molecule. In some embodiments, the acceptor molecule is a hydrophobic molecule, a small molecule, or both. In some embodiments, the donor molecule comprises an acyl group, CoA, or both. In some embodiments, the polynucleotide encodes a protein characterized by acyltransferase catalytic activity. In some embodiments, the polynucleotide encodes a protein characterized by being capable of transferring an acyl group to a cannabinoid. In some embodiments, the polynucleotide encodes a protein characterized by having a catalytic activity of acylating a cannabinoid. In some embodiments, the acyltransferase (AT) is an alcohol acyltransferase (AAT). In some embodiments, the polynucleotide encodes an AT enzyme. In some embodiments, the polynucleotide encodes an AAT enzyme.

In some embodiments, the AAT is an AAT derived from Helichrysum umbraculigerum. As used herein, the term “AAT” encompasses any enzyme derived from H. umbraculigerum and having or characterized by having an activity as described herein.

According to some embodiments, there is provided an artificial nucleic acid molecule comprising the polynucleotide disclosed herein.

In some embodiments, the artificial vector comprises a plasmid. In some embodiments, the artificial vector comprises or is an agrobacterium comprising the artificial nucleic acid molecule. In some embodiments, the artificial vector is an expression vector. In some embodiments, the artificial vector is a plant expression vector. In some embodiments, the artificial vector is for use in expressing AAT encoding nucleic acid sequence as disclosed herein. In some embodiments, the artificial vector is for use in heterologous expression of AAT encoding nucleic acid sequence as disclosed herein in a cell, a tissue, or an organism.

Expression of a polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation, and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the artificial vector comprises a polynucleotide encoding a protein comprising an amino acid sequence as described herein.

According to some embodiments, there is provided a protein encoded by: (a) the polynucleotide disclosed herein; (b) the artificial vector disclosed herein; or the plasmid or agrobacterium disclosed herein.

In some embodiments, the protein is encoded by a polynucleotide comprising or consisting of SEQ ID Nos.: 1-15.

In some embodiments, the protein comprises an amino acid sequence with at least 90%, at least 92%, at least 93%, at least 95%, at least 97%, or at least 99% homology or identity to any one of SEQ ID Nos.: 16-30, or any value and range therebetween. Each possibility represents a separate embodiment of the invention. In some embodiments, the protein comprises an amino acid sequence with 90-100%, 93-100%, 95-100%, or 97-100% homology or identity to any one of SEQ ID Nos.: 16-30. Each possibility represents a separate embodiment of the invention.

In some embodiments, the protein is an isolated protein.

As used herein, the terms “peptide”, “polypeptide” and “protein” are interchangeable and refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the terms “isolated protein” refers to a protein that is essentially free from contaminating cellular components, such as carbohydrate, lipid, or other proteinaceous impurities associated with the nucleic acid in nature. Typically, a preparation of an isolated protein contains the protein in a highly purified form, e.g., at least about 80% pure, at least about 90% pure, at least about 95% pure, greater than 95% pure, or greater than 99% pure. In some embodiments, the isolated protein is a synthesized protein. Synthesis of protein is well known in the art and may be performed, for example, by heterologous expression in a transformed cell, such as exemplified herein.

In some embodiments, the protein comprises or consists of the amino acid sequence:

(SEQ ID NO: 16)
MATQVKTEEKHLKVEIINKTYVKPETPLGRKECQLVTFDLPYIAFYYNQK
LIIYKGGVEEFEDTVEKLKDGLKVVLGEFHQLAGKLDKDDDGVFKVVYDD
DMDGVEVLSAVAEDTATADLMDEEGTIKLKELVPYNSVLNIEGLHRPLLS
IQITKLKDGLVLGCAFNHAILDGTSTWHFMSSWAQICSGSKSISAAPFLD
RTQARNTRVKLDLTPPAQTNGNSNGDTNGDASATKPPAPAPLREKIFKFS
ESAIDKIKAKINANPPEGSTKPFSTFQSLSTHIWHAVTRARNLKPEDYTV
FTVFADCRKRVDPPMPDSYFGNLIQAIFTVTAAGLLQANPPEFAASMIQK
AIDMHDAKAIEARNKEWESNPIIFQYKDAGVNCVAVGSSPRFKVYDVDFG
FGKPESVRSGANNRFDGMVYLYQGKSGGRSIDVEISLDASAMGNLEKDKE
FLIQE.

In some embodiments, the protein comprises an amino acid sequence at least 87%, at least 92%, at least 95%, or at least 99% homology or identity to SEQ ID NO: 16, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 16. Each possibility represents a separate embodiment of the invention.

In some embodiments, the protein comprises or consists of the amino acid sequence:

(SEQ ID NO: 17)
MASLPLLTVLEQSHVSPPPATVVDKSLSLTFFDFLWLTQPPIHNLFFYEF
SIDETQFVETIVPSLKNSLSITLQHFYPFAGNLILFPDNKRPEIRYVEGD
YVMVTFAKSSLDFNELVGNHPRDCDQFYDLIPPLGESVKTSEFRKIPLFS
VQVTFFPQKGVSIGMTNHHSLGDASTRFCFLNAWTSISRSSSDESFLANG
TKPFYDRVISNPKLDQSYLKFSKIDTLYEKYQPLSLSRPSNKLRGTFILT
RKILNELKKSVSIKLPTLSYVSSFTVACGYIWSCIAKSRNDDLQLFGFTI
DCRARLDPPVPSTYFGNCVGGCMAMAKTTLLTEDDGFITAAKLLGESLHK
TLTESGGIVKDIEVFEDLFKDGLPTTMIGVAGTPKLKFYETDFGWGNPKK
VETISIDYNMSISMNACRESKDDLEIGVCLMNTEMEAFVRLFDEGLESY
V.

In some embodiments, the protein comprises an amino acid sequence with at least 72%, at least 80%, at least 89%, at least 95%, or at least 99% homology or identity to SEQ ID NO: 17, or any value and range therebetween. Each possibility represents a separate embodiment of the invention. In some embodiments, the protein comprises an amino acid sequence with 72% to 100%, 80% to 100%, or 90% to 100% homology or identity to SEQ ID NO: 17. Each possibility represents a separate embodiment of the invention.

In some embodiments, the protein comprises or consists of the amino acid sequence:

(SEQ ID NO: 18)
MGSENVHKIMKINITKSSFVQPSKPTVLPTNHIWTSNLDLVVGRIHILTV
YFYRPNGASNFFDPIVMKKALADVLVSFYPMAGRISKDDNGRVVINCNDE
GVLFVEAESDSTLDDFGEFTPSPELRQLTPTIDYSGDISTYPLFFAQVTH
FKCGGVGFGCGVFHTLADGLSSIHFINTWSDMARGLSIAIPPFTDRTLLR
AREPPTPTFDHVEYHLPPSMKTTSQTNKSRKPSTAMLKLTLDQLNALKAA
AKNEGGNTNYSTYEILAAHLWRCACKARGLPDDQLTKLYVATDGRSRLSP
QLPPGYLGNVVFTATPVAKSADLTTQPLSNAASLIRTTLTKMDNDYLRSA
IDYLEVQPDLSALIRGPSYFASPNLNINTWTRLPVHDADFGWGRPVFMGP
AVILYEGTIYVLPSPNNDRSMSLAVCLDADEQPSFEKFLYDF.

In some embodiments, the protein comprises an amino acid sequence with at least 90%, at least 92%, at least 95%, at least 97%, or at least 99% homology or identity to SEQ ID NO: 18, or any value and range therebetween. Each possibility represents a separate embodiment of the invention. In some embodiments, the protein comprises an amino acid sequence with 90% to 100%, 95% to 100%, or 97% to 100% homology or identity to SEQ ID NO: 18. Each possibility represents a separate embodiment of the invention.

In some embodiments, the protein comprises or consists of the amino acid sequence:

(SEQ ID NO: 19)
MPSSSSSPSSTADSVTIISKCTVYPHMKNSTPESLQLSVSDLPMLSCQYI
QKGVLLSQPPPNHTNNIISHLKLSLSKTLSHFPPLAGRLSTDSHGHVSII
CNDSGVEFVHSTANHLHTHQILPLNSDVHPCFKTFFAFDKTLSYAGHHQP
IAAVQVTELADGLFIGCTVNHAVVDGTSFWNFFNTFAEITKGCQKVTNLP
DFSRENVFISPVVLPLPSGGPSATFSGDEPLRERIIHFSRDAILKMKFRA
NNPLWRQPQNSDLDDTEIYGKVCNDINGKVNGAFKPKSEISSFQSLCGQL
WRAVTRARKENDPIKTTTFRMAVNCRHRLDPKVDKLYFGNLIQSIPTVAS
VGELLSHDLSWAANELHQNVVAHDNATVRRGVKDWENNPKLFPLGNFDGA
MITMGSSPRFPMYNNDFGWGRPMAVRSGKANKFDGKISAFPGRDGDGSVD
LEVVLAPETMACLERDHEFMQYVS.

In some embodiments, the protein comprises an amino acid sequence with at least 86%, at least 92%, at least 95%, or at least 99% homology or identity to SEQ ID NO: 19, or any value and range therebetween. Each possibility represents a separate embodiment of the invention. In some embodiments, the protein comprises an amino acid sequence with 86% to 100%, 90% to 100%, or 95% to 100% homology or identity to SEQ ID NO: 19. Each possibility represents a separate embodiment of the invention.

In some embodiments, the protein comprises or consists of the amino acid sequence:

(SEQ ID NO: 20)
MKWFFITHKATQRCLNSKQFHLHGGSNFVSGNRCFLASHSMERPKFMLIP
YYPYQIRSLNSSHRYSSTSPSGSPHSFLNGTKNENYTKKVDLEIISREII
KPASPTPHHLRNFNLSLLDQIVFDCYTPVILFIPNSNKATVTDVMIKRLK
HLKETLSRILSQFYPFAGEVKDRLHIECNDKGVNYIEAQINETLEEFLCH
PDNEKARELMPESPHVQESAIGNYAMGIQINIFSCGGIGLSMSMAHKIMD
FYTYTIFMKAWAAAVRGSPDTIISPSFVASEVFPNDPSQEDSIPIELKSS
NLLSTKRFEFDPTALALLKGQVVASGSPPQRGPSRMEATTAVIWKAAAKA
ASTVRRFDPKSPHALALPVNIRKRASPALPDNSIGNIVMRGIAICFPESQ
PDLPTLMGKVRESIAKLNSDYIESLKGEKGHETVNKMLKELKLRTNMTKV
GGKFVASCIFNSGIYELDFGWGKPIWFYVVNPGSDSCVVLTDTLKGGGVE
ATITLPPDEMEIFERDHELLSYTTINPSPLRFLDH.

In some embodiments, the protein comprises an amino acid sequence with at least 59%, at least 65%, at least 75%, at least 85%, at least 90%, or at least 99% homology or identity to SEQ ID NO: 20, or any value and range therebetween. Each possibility represents a separate embodiment of the invention. In some embodiments, the protein comprises an amino acid sequence with 59% to 100%, 70% to 100%, 80% to 100%, 90% to 100%, 95% to 100%, or 97% to 100% homology or identity to SEQ ID NO: 20. Each possibility represents a separate embodiment of the invention.

In some embodiments, the protein comprises or consists of the amino acid sequence:

(SEQ ID NO: 21)
MEVPDQFHLNILEQCHVSPSPNSIIPSFSLPLTFLDIPWLFYPSNQTLFF
FPEPPPKTTIITTLKQSLSLTLHHFHPLAGNLSLPSPPAEPHIVYTKNDS
IALTIAQTNTNIHHLSCNHPRSVKNLYSLLPKLPSPSMSRETHVGLVIPL
LTIQITVFADLGYSIGVTMQHAAVDERTFDQFMKCWASVCTSLLKNDSLF
TFKSTPWYDRSVIIDPKSLKTTFLKQWWNRSNSLNESHDQENDDHDLVLA
TFVLSSLDINMIKNHILAKCKMINEDPPLHLSPYVSACAYLWKCLIKIQE
THDSIKGGPLYLGFNAGGITRLGYDIPSTYFGNCIAFGRCKAFESELLGD
NGIVFAAKSIGKEIKRLDKDVLGGANKWISDWDELTIRLLGSPKVDSYGM
DFGWGKVEKVEKISSISNHGRVNVISLSGCKDFKGGIEIGVVLSVAKMNV
FTSLFHGGLMEFAY.

In some embodiments, the protein comprises an amino acid sequence with at least 71%, at least 80%, at least 90%, at least 95%, or at least 99% homology or identity to SEQ ID NO: 21, or any value and range therebetween. Each possibility represents a separate embodiment of the invention. In some embodiments, the protein comprises an amino acid sequence with 71% to 100%, 80% to 100%, 87% to 100%, or 95% to 100% homology or identity to SEQ ID NO: 21. Each possibility represents a separate embodiment of the invention.

In some embodiments, the protein comprises or consists of the amino acid sequence:

(SEQ ID NO: 22)
MKNKNPTSVIREALAKVLVFYYPFAGRLKEGPARKLMVDCSGEGVLFIEA
EADVTLKQFGDALQPPFPCLEELLYDVPGSTGILDTPLLLIQVTRLLCGG
FIFALRLNHTMSDAAGLVQFMTGLGEMAQGASRPSTLPVWQRELLFARDP
PRVTCTHHEYTEVEDTNGTIIPLDDMAHKSFFFGPSEISALRRFVPSYLK
KCSTFEVLTACLWRCRTIALQPDPEEEMRMICIVNARGKFNPPLLPKGYY
GNGFAIPVAISTAGDLSSKPLGHALELVMKAKSNVTEEYMRSVADLMVIK
GRPHYTVVRSYLVSDVTHAGFDVVDFGWGKASYGGPAKGGVGAIPGVVTF
FIPFTNHKGESGIVLPICLPSAAMDKFVEELNKMLVPDNNEQVLREHKLL
VLARL.

In some embodiments, the protein comprises an amino acid sequence with at least 88%, at least 92%, at least 95%, or at least 99% homology or identity to SEQ ID NO: 22, or any value and range therebetween. Each possibility represents a separate embodiment of the invention. In some embodiments, the protein comprises an amino acid sequence with 88% to 100%, 92% to 100%, 97% to 100%, or 99% to 100% homology or identity to SEQ ID NO: 22. Each possibility represents a separate embodiment of the invention.

In some embodiments, the protein comprises or consists of the amino acid sequence:

(SEQ ID NO: 23)
MAQIDTPLTFKVRRHAPELIAPAKPTPRELKPLSDIDDQEGLRFHIPVIQ
FYRSDPKMKNKNPASVIREALAKVLVFYYPFAGRLKEGPARKLMVDCSGE
GVLFIEAEADVTLKQFGDALQPPFPCLEELLYDVPGSTGVLDTPLLLIQV
TRLLCGGFIFALRLNHTMSDAPGLVQFMTGLGEMAQGASRPSTLPVWQRE
LLLARDPPRVTCTHHEYTEVEDTKGTIIPLDDMAHKSFFFGPSEISALRR
FVPSYLKKCSTFEVLTACLWRCRTIALQPDPEEEMRIICIVNARGKFNPP
LPKGYYGNGFAFPVAISTAGDLSSKPLGHALELVMKAKSDVTEEYMRSIA
DLMVIKGRPHFTVVRSYLVSDVTHAGFDVVDFGWGKAAYGGPAKGGVGAI
PGVASFYIPFTNHKGESGIVLPICLPSAAMDKFVEELNKMLVPDNNEQVL
REHKLLVLARL.

In some embodiments, the protein comprises an amino acid sequence with at least 91%, at least 93%, at least 95%, or at least 99% homology or identity to SEQ ID NO: 23, or any value and range therebetween. Each possibility represents a separate embodiment of the invention. In some embodiments, the protein comprises an amino acid sequence with 91% to 100%, 93% to 100%, 95% to 100%, or 97% to 100% homology or identity to SEQ ID NO: 23. Each possibility represents a separate embodiment of the invention.

In some embodiments, the protein comprises or consists of the amino acid sequence:

(SEQ ID NO: 24)
MEIQVINYSSKLVKPLTPTPTANRYYNISFTDELVPTIYVPLILYYATPK
NPNGDHFENICDRLEESLSKTLSDFYPLAARFIRKLSLIDCNDQGVLFVL
GNVNIRLSDVTGLGLTFKTSVLNDFLPCEIGGADEVDDPMLCVKVTTFEC
GGFAIGMCFSHRLSDMGTMCNFINNWAARTIGEYDNEKHTPIFNSPLYFP
QRGLPELDLKVPRSSIGVKNAARMFHFNGKAISSMREVFGVDENGSRRLS
KVQLVVALLWKAFVRIDDVNDGQSKASFLIQPVGLRDKVVPPLPSNSFGN
EWGLATSQLGPGEGHKIGFQEYFYILRESIKKRARDCAKILTHGEEGYGV
VIDPYLESNQKIADNGTNFYLFTCWCKFSFYEADFGCGKPIWASTGKFPV
QNLVIMMDDNEGDGVEAWVHLDDKRMNELEQDPDVKLYACNLA.

In some embodiments, the protein comprises an amino acid sequence with at least 73%, at least 85%, at least 92%, at least 95%, or at least 99% homology or identity to SEQ ID NO: 24, or any value and range therebetween. Each possibility represents a separate embodiment of the invention. In some embodiments, the protein comprises an amino acid sequence with 73% to 100%, 80% to 100%, 90% to 100%, or 95% to 100% homology or identity to SEQ ID NO: 24. Each possibility represents a separate embodiment of the invention.

In some embodiments, the protein comprises or consists of the amino acid sequence:

(SEQ ID NO: 25)
MKLAVKESVIVKPSKTTPCQQIWTSNLDLVVGRIHILTVYLYRPNGSSNF
FDSMVLKKALADVLVSFFPVAGRLDKDGDGRVVIDCNGEGVLFVEAEADC
CIDDFGEITPSPELRRLVPTVDYSGDMSSYPLFITQVTRFKCGGVSLGCG
LHHTLSDGLSALHFINTWSDVARGLSVAIPPFIDRSLLRARDPPSPVFDH
IEYHPPPSLITPLQNQKNASHSRSASTLILRLTLHQINNLKSKAKGDGSM
YHSTYEILAAHLWRCACKARGLANDQPTKLYVATDGRSRLIPPLPPGYLG
NVVFTATPVAKSGDFESESLAETARRIRSELGKMNDEYLRSAIDYLESVS
DISTLVRGPTYFASPNLNVNSWTRLPIYESDFGWGRPIFMGPASILYEGT
IYIIPSPSGDRSVSLAVCLDPDHMALFKECLYVF.

In some embodiments, the protein comprises an amino acid sequence with at least 83%, at least 88%, at least 92%, at least 95%, or at least 99% homology or identity to SEQ ID NO: 25, or any value and range therebetween. Each possibility represents a separate embodiment of the invention. In some embodiments, the protein comprises an amino acid sequence with 83% to 100%, 88% to 100%, 94% to 100%, or 97% to 100% homology or identity to SEQ ID NO: 25. Each possibility represents a separate embodiment of the invention.

In some embodiments, the protein comprises or consists of the amino acid sequence:

(SEQ ID NO: 26)
MKLAVKESVIVKPSKTTPCQQIRTSNLDLVAGRIHILVVFFYRPNGSSNF
FDSLVLKKALADVLVPFFPVAGRFSEDGDGRVVIDCNGEGVLFVESEADC
CIDDFGEITLSPELQQLVPTVDYSGDMSSYPLFIAQVTRFKCGGVSLGWG
LHHTLLDGLSALHFVNTWGDVARGLSVAIQPFIDRSLLRARDPPTPVFDH
IEYHPPPSLITPLQNQKNASHSRSASTLILQLTPDQIKNLKSKAKGDGSM
YHSTYEILAAHLWRCACKARGLANDQPTKLYVAANGRSRLIPPLPPGYLG
NVVFNATHVAKSGDFESESLAETARRIHCELGKMNDEYFRSAIDYLESVD
DISTLVKGPTYFASPNLNVYSWIGIPIYACDFGWGQPIFMRPASFLYDGS
IYIIPSPSGDRSVLLAVCLDPDHMDLFKECLYAF.

In some embodiments, the protein comprises an amino acid sequence with at least 76%, at least 84%, at least 92%, or at least 99% homology or identity to SEQ ID NO: 26, or any value and range therebetween. Each possibility represents a separate embodiment of the invention. In some embodiments, the protein comprises an amino acid sequence with 76% to 100%, 83% to 100%, 90% to 100%, or 95% to 100% homology or identity to SEQ ID NO: 26. Each possibility represents a separate embodiment of the invention.

In some embodiments, the protein comprises or consists of the amino acid sequence:

(SEQ ID NO: 27)
MVMISKLLRLGRRKLHTIVSRDTIRPSSPTPSHSKTYNLSLLDQIAVNSY
VPIVAFYPSSNVCRSSDDKTLELKNSLSKILTHYYPFAGRMKKNRPTVVD
CNDEGVEFVEARNTNSLSDFLQQSEHEDLDQLFPDDCVWFKQNLKGSIND
ANNSSVCPLSIQVNHFACGGVAVATSLRHKIGDGSSALNFIKHWAAVTSH
SRAGNHQIDATSPIINPHFISYPTRTFKLPDRSPYIPPSDVVSKSFVFPN
TNIKDLQAKVVTMTMGSRQPIVNPTRADVVSWLLHKCVVAAATKRISGNF
KESCVISPLNLRNKLEEPLPETSIGNIFYLITFPISNNHGDLMPDDFISQ
LRLGIRKFQNIRNLETALRTVEEMISETFILGTAESMDTSYVYSSIRGFP
MYDIDFGWGKPVKVTVGGALKNLSILMDTPDVNGIEALVSLDKQDMKILL
NDPELLAFCL.

In some embodiments, the protein comprises an amino acid sequence with at least 60%, at least 70%, at least 80%, at least 90%, or at least 95% homology or identity to SEQ ID NO: 27, or any value and range therebetween. Each possibility represents a separate embodiment of the invention. In some embodiments, the protein comprises an amino acid sequence with 60% to 100%, 70% to 100%, 80% to 100%, or 90% to 100% homology or identity to SEQ ID NO: 27. Each possibility represents a separate embodiment of the invention.

In some embodiments, the protein comprises or consists of the amino acid sequence:

(SEQ ID NO: 28)
MSTSDKMKITIRESSMIKPSKPTPDQRIWNSNLDLVVGRIHILTLYFFRP
NGSSDFFDSEVLKQSLADVLVSFFPMAGRLGLDGDGRVEINCNGEGVLFV
EAEADCSIDDFGEITPSPELRRLAPTVDYSGDISSYPLVITQVTHFKCGG
VSLGCGLHHTLSDGLSSLHFINTWSDVTRGLPVAIPPFVDRTVLRARDPP
TVVFDHVEYHTPPSMTSSLDKDKPQSEDVHVSTSMLRLTLDQINALKAKG
KGDGIVYHSTYEILAAHLWRCACKARGLLNDQMTKLYVATDGRSRLIPPL
PPGYLGNVVFTATPIAKSGELQQEPLATTARKIHTELAKMDDKYLRSALD
YLESQQDLSALIRGPAYFACPNLNINSWTRLPIYDADFGWGRPIFMGPAS
ILYEGTIYIIPSPSGDRSVSLAVCLDPSHMPLFQKYLYEL.

In some embodiments, the protein comprises an amino acid sequence with at least 85%, at least 89%, at least 92%, at least 95%, or at least 99% homology or identity to SEQ ID NO: 28, or any value and range therebetween. Each possibility represents a separate embodiment of the invention. In some embodiments, the protein comprises an amino acid sequence with 85% to 100%, 90% to 100%, 93% to 100%, or 96% to 100% homology or identity to SEQ ID NO: 28. Each possibility represents a separate embodiment of the invention.

In some embodiments, the protein comprises or consists of the amino acid sequence:

(SEQ ID NO: 29)
MVNVEIISNEYIKPSSPTPPHLKIYNLSILDQLIPAPYAPIILYYPNQDH
INDFEVHERLKLLKDSLSKTLTRFYPLAGTIKGDLSIDCNDIGAYFAVAH
VNTRLDVFLNHPDLDLINCFLPRGPYLNGSSEGSCVSNVQVNIFECCGIA
ISLCISHKILDGAALSTFLKAWAGTSYGSKEVVYPNMSAPSLFPAKDLWL
KDSSMVMFGSLFKMGKCSTKRFVFDSSKLSFLKAKASLNGLKDPTRVEVV
SALLWKCIMAASEENTGSWKPSLLSHVVNLRKRLVSTLSEDSIGNLIWLA
SAECRTNAQSRLSDLVEKVRDSVSKINSEFVKKIQGDKGTKVMEESLKSM
KDCADYIGFTSWCKMGFYDVDFGWGKPVWVCGSVCEGSPVFMNFVILMDT
KYGDGIEAWVSLDEHEMHILKHNPELLEYASIDPSPLQMNK.

In some embodiments, the protein comprises an amino acid sequence with at least 82%, at least 85%, at least 92%, at least 95%, or at least 99% homology or identity to SEQ ID NO: 29, or any value and range therebetween. Each possibility represents a separate embodiment of the invention. In some embodiments, the protein comprises an amino acid sequence with 82% to 100%, 85% to 100%, 90% to 100%, or 93% to 100% homology or identity to SEQ ID NO: 29. Each possibility represents a separate embodiment of the invention.

In some embodiments, the protein comprises or consists of the amino acid sequence:

(SEQ ID NO: 30)
MGTIYQSPMIKSSTPKIIEDLKVIIHDTFTIFPPHETEKRSMFLSNIDQV
LTFNVETVHFFAANPDFPPQVVAEKLKLALSKALVPYDFLAGRLKLNHES
QRFEFDCNGAGARFVVGSSEFELGEIGDLVYPNPGFRQLVQKSYDNLELH
EKPLCILQLTSFKCGGFALGVATNHATFDGLSFKTFLQNLGSLAADQPLA
VDPCNDRHLLAARSPPKVQFDHPELLKIPTGTDIPNPTVFDCPESQLDFK
IFNLTSDDIAHLKTKAKDGPGSTNAKITGFNVVAAHVWRCKALSSGSEYD
PERVSTVLYAVDIRSRLNLPLSLAGNAVLSAYASAKCKEIEEGPLSRLVE
MVTEGTNRMTGEYARSVIDWGEVNKGFPNGEFLISSWWRLGFADVEYPWG
KPRYSCPVVYHRKDIILLFPDIVGADNNNEVNVLVALPGKEMEKFETLFH
KFLA.

In some embodiments, the protein comprises an amino acid sequence with at least 88%, at least 92%, at least 95%, or at least 99% homology or identity to SEQ ID NO: 30, or any value and range therebetween. Each possibility represents a separate embodiment of the invention. In some embodiments, the protein comprises an amino acid sequence with 88% to 100%, 90% to 100%, 93% to 100%, or 95% to 100% homology or identity to SEQ ID NO: 30. Each possibility represents a separate embodiment of the invention.

In some embodiments, the protein comprises an amino acid sequence set forth in SEQ ID Nos: 19 or 30.

The terms “homology” or “identity”, as used interchangeably herein, refer to sequence identity between two amino acid sequences or two nucleic acid sequences, with identity being a stricter comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percentage identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

According to some embodiments, there is provided a transgenic cell comprising: (a) the polynucleotide disclosed herein; (b) the artificial nucleic acid molecule disclosed herein; (c) the plasmid or agrobacterium disclosed herein; (d) the isolated protein disclosed herein; or any combination thereof.

As used herein, the term “transgenic cell” refers to any cell that has undergone human manipulation on the genomic or gene level. In some embodiments, the transgenic cell has had exogenous polynucleotide, such as an isolated DNA molecule as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous polynucleotide (e.g., the isolated DNA molecule disclosed herein) or vector is stably integrated into the cell. In some embodiments, the transgenic cell expresses a polynucleotide of the invention. In some embodiments, the transgenic cell expresses a vector of the invention. In some embodiments, the transgenic cell expresses a protein of the invention. In some embodiments, the transgenic cell, is a cell that is devoid of a polynucleotide of the invention that has been transformed or genetically modified to include the polynucleotide of the invention. In some embodiments, CRISPR technology is used to modify the genome of the cell, as described herein.

In some embodiments, the cell is a unicellular organism, a cell of a multicellular organism, and a cell in a culture.

In some embodiments, a unicellular organism comprises a fungus or a bacterium.

In some embodiments, the fungus is a yeast cell.

In some embodiments, the cell is an insect cell. In some embodiments, the cell comprises an insect cell line.

Types of insect cell lines suitable for transformation and/or heterologous expression are common and would be apparent to one of ordinary skill in the art. Non-limiting examples of such insect cell lines include, but are not limited to, Sf-9 cells, SR+ Schneider cells, S2 cells, and others.

According to some embodiments, there is provided an extract derived from a transgenic cell disclosed herein, or any fraction thereof.

In some embodiments, the extract comprises the polynucleotide of the invention, an isolated DNA molecule as disclosed herein, an isolated protein as disclosed herein, or any combination thereof.

According to some embodiments, there is provided a homogenate, lysate, extract, derived from a transgenic cell disclosed herein, any combination thereof, or any fraction thereof.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar, or nonpolar solvent), liquid chromatography mass spectrometry, or others.

According to some embodiments, there is provided a transgenic plant, a transgenic plant tissue or a plant part. In some embodiments, there is provided a transgenic plant, or any portion, seed, tissue, or organ thereof, comprising at least one transgenic plant cell of the invention. In some embodiments, the transgenic plant, transgenic plant tissue or plant part, comprises: (a) the polynucleotide disclosed herein; (b) the artificial disclosed herein; (c) the plasmid or agrobacterium disclosed herein; (d) the isolated protein of the invention; (e) the transgenic cell disclosed herein; or any combination thereof.

In some embodiments, the transgenic plant, transgenic plant tissue, or plant part consists of transgenic plant cells of the invention. In some embodiments, the transgenic plant, transgenic plant tissue, or plant part comprises at least: 20%, 25%, 30%, 35%, 40%, 45%, 50%, 55%, 60%, 65%, 70%, 75%, 80%, 85%, 90%, 95%, 97%, or 99% transgenic cells of the invention, or any value and range therebetween. Each possibility represents a separate embodiment of the invention. In some embodiments, the transgenic plant, transgenic plant tissue, or plant part comprises 20%-50%, 20%-60%, 20%-70%, 20%-80%, 20%-90%, or 20%-100% transgenic cells of the invention. Each possibility represents a separate embodiment of the invention.

In some embodiments, the transgenic plant, transgenic plant tissue, or plant part is or derived from a Cannabis sativa plant. In some embodiments, the transgenic plant is a C. sativa plant.

In some embodiments, the transgenic plant, transgenic plant tissue, or plant part is or derived from hemp. In some embodiments, C. sativa comprises or is hemp.

According to some embodiments, there is provided a composition comprising any one of the herein disclosed: (a) polynucleotide of the invention (for example, an isolated DNA molecule); (b) artificial vector; (c) plasmid or agrobacterium; (d) isolated protein of the invention; (e) transgenic cell; (f) extract; (g) transgenic plant tissue or plant part; and (h) any combination of (a) to (g), and an acceptable carrier.

As used herein, the term “carrier”, “excipient”, or “adjuvant” refers to any component of a composition, e.g., pharmaceutical or nutraceutical, that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods of Synthesis

According to some embodiments, there is provided a method for synthesizing an acylated cannabinoid.

According to some embodiments, there is provided a method for acylating a cannabinoid.

According to some embodiments, there is provided a method for synthesizing an acylated cannabinoid or a precursor thereof.

According to some embodiments, there is provided a method for acylating a cannabinoid or a precursor thereof.

According to some embodiments, the method comprises the steps: (a) providing a cell comprising an artificial vector comprising a nucleic acid sequence having at least 87%, at least 89%, at least 92%, at least 95%, at least 97%, or at least 99% homology or identity to any one of SEQ ID Nos.: 1-15, or any combination thereof, or any value and range therebetween; and (b) culturing the cell from step (a) such that a protein encoded by the artificial vector is expressed. Each possibility represents a separate embodiment of the invention.

According to some embodiments, the method comprises the steps: (a) providing a cell comprising an artificial vector comprising a nucleic acid sequence having at least 87%, at least 89%, at least 92%, at least 95%, at least 97%, or at least 99% homology or identity to any one of SEQ ID Nos.: 1-15, or any combination thereof, or any value and range therebetween; and (b) culturing the cell from step (a) such that a protein encoded by the artificial vector is expressed. Each possibility represents a separate embodiment of the invention.

According to some embodiments, the method comprises contacting a cannabinoid with an effective amount of a protein comprising an amino acid sequence with at least 91%, at least 93%, at least 95%, at least 99%, or 100% homology or identity to any one of SEQ ID Nos.: 16-30, or any value and range therebetween. Each possibility represents a separate embodiment of the invention.

According to some embodiments, the method comprises contacting a cannabinoid with an effective amount of a protein comprising an amino acid sequence with at least 90%, at least 93%, at least 95%, at least 99%, or 100% homology or identity to any one of SEQ ID Nos.: 16-30, or any value and range therebetween. Each possibility represents a separate embodiment of the invention.

According to some embodiments, the method comprises contacting a cannabinoid precursor with an effective amount of a protein comprising an amino acid sequence with at least 91%, at least 93%, at least 95%, at least 99%, or 100% homology or identity to any one of SEQ ID Nos.: 16-30, or any value and range therebetween. Each possibility represents a separate embodiment of the invention.

According to some embodiments, the method comprises contacting a cannabinoid precursor with an effective amount of a protein comprising an amino acid sequence with at least 90%, at least 93%, at least 95%, at least 99%, or 100% homology or identity to any one of SEQ ID Nos.: 16-30, or any value and range therebetween. Each possibility represents a separate embodiment of the invention.

In some embodiments, the cannabinoid is or comprises CBDA, CBGA, HeliCBGA, or any combination thereof.

In some embodiments, a cannabinoid precursor is or comprises olivetolic acid (OA).

According to some embodiments, there is provided a method for obtaining an extract from a transgenic cell or a transfected cell.

In some embodiments, the method comprises culturing a transgenic cell or a transfected cell in a medium and extracting the transgenic cell or the transfected cell.

In some embodiments, the method comprises the steps: (a) culturing a transgenic cell or a transfected cell in a medium; and (b) extracting the transgenic cell or the transfected cell, thereby obtaining an extract from the transgenic cell or the transfected cell.

In some embodiments, the transgenic cell or the transfected cell comprises an artificial vector comprising a nucleic acid sequence having at least 87%, at least 89%, at least 92%, at least 95%, at least 97%, at least 99%, or 100% homology or identity to any one of SEQ ID Nos.: 1-15, or any combination thereof, or any value and range therebetween. Each possibility represents a separate embodiment of the invention.

In some embodiments, the transgenic cell or the transfected cell comprises the polynucleotide of the invention or a plurality thereof, as disclosed herein.

In some embodiments, the transgenic cell or the transfected cell comprises the artificial nucleic acid molecule or vector as disclosed herein.

In some embodiments, the cell is a transgenic cell, or a cell transfected with an isolated DNA molecule as disclosed herein.

In some embodiments, the culturing comprises supplementing the cell with an effective amount of a cannabinoid or a precursor thereof. In some embodiments, the supplementing is via the growth or culture medium wherein the cell is cultured.

In some embodiments, the culturing comprises supplementing the cell with an effective amount of an acyl donor or a donor molecule comprising an acyl group. In some embodiments, an acyl donor comprises a CoA group. In some embodiments, an acyl donor comprises: Butyryl CoA, Hexanoyl CoA, iso-Valeryl CoA, Acetyl COA, iso-Butyryl CoA or any combination thereof.

As used herein, the terms “acyl donor” or “donor molecule comprising an acyl group” are interchangeable.

In some embodiments, the method further comprises a step preceding step (a), comprising introducing or transfecting the cell with the artificial nucleic acid molecule or vector, disclosed herein.

Method for introducing or transfecting a cell with an artificial nucleic acid molecule or vector are common and would be apparent to one of ordinary skill in the art.

In some embodiments, introducing or transfecting comprises transferring an artificial nucleic acid molecule or vector comprising the polynucleotide disclosed herein into a cell; or modifying the genome of a cell to include the polynucleotide disclosed herein. In some embodiments, the transferring comprises transfection. In some embodiments, the transferring comprises transformation. In some embodiments, the transferring comprises lipofection. In some embodiments, the transferring comprises nucleofection. In some embodiments, the transferring comprises viral infection.

As used herein, the terms “transfecting” and “introducing” are interchangeable.

In some embodiments, contacting is in a cell-free system.

Types of suitable cell-free systems for utilizing any one of: the polynucleotide of the invention or a plurality thereof, as disclosed herein, and the isolated protein of the invention, or a plurality thereof, would be apparent to one of ordinary skill in the art.

In some embodiments, the method further comprises a step preceding step (b), comprising separating the cultured transgenic cell or the cultured transfected cell from the medium.

Method for separating cell from a medium are common and may include, but not limited to, centrifugation, ultracentrifugation, or other, as would be apparent to one of ordinary skill in the art.

According to some embodiments, there is provided an extract of a transgenic cell, or a transfected cell obtained according to the herein disclosed method.

According to some embodiments, there is provided a medium or a portion thereof separated from a cultured transgenic cell or a cultured transfected cell, obtained according to the herein disclosed method.

According to some embodiments, there is provided a composition comprising: (a) the extract disclosed herein; (b) the medium disclosed herein or a portion thereof; or (c) any combination of (a) and (b), and an acceptable carrier, as described herein.

In some embodiments, a portion comprises a fraction or a plurality thereof.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

### Chemicals

CBGA, cannabidiolic acid (CBDA), (−)-trans-Δ⁹-tetrahydrocannabinolic acid (Δ⁹-THCA), cannabichromenic acid (CBCA), butyryl-CoA, iso-Butyryl-CoA, hexanoyl-CoA, iso-valeryl-CoA, acetyl-CoA, butyric acid, hexanoic acid, ±2-methyl butyric acid, phenylalanine, and hexanoic-D₁₁ acid (D>98%) were purchased from Sigma-Aldrich (Rehovot, Israel). Butyric-D₅ acid (D>98%), ±2-methyl butyric-D₉ acid (D>99%), and iso-valeric-D₉ acid (D>98%) were purchased from C/D/N isotopes (Quebec, Canada). Phenylalanine-D₅ (D>98%) and phenylalanine-¹³C₉, ¹⁵N₁ (¹³C, ¹⁵N>99%) were synthesized by Cambridge Isotope Laboratories (Andover, MA). HeliCBGA (NP009525, 90%) was purchased from Analyticon Discovery GmbH (Potsdam, Germany). Olivetolic acid (OA) was purchased from Cayman Chemical (Ann Arbor, MI, USA).

### Feeding Experiments

All the feeding solutions were prepared as aqua solutions of 0.5 mg ml⁻¹ of the precursor. The pH of the short- and medium chain fatty acid (FA) solutions was adjusted to be in the range of 5.5-6.0. The phenylalanine feeding experiments was performed on leaves from young mother plants excised by cutting at the proximal side of the pedicel with scissors under water (to avoid air penetration into the pedicel, which may influence the feeding efficiency), leaving attached 1-2 cm of the pedicel. For the short-chain FA feeding experiments, 10 cm young cuttings were obtained from mother plants. The lower leaves were removed, leaving 4-5 leaves on each stem, and the stem was peeled to increase the intake of the labeled solutions. Three to four leaves or the young cuttings were immersed into Eppendorf tubes with 1.8 ml of the aqua solutions [DDW (control), unlabeled or labeled precursors, each group consisted of a minimum of three biological replicates]. All feeding experiments were performed in a controlled environment for 48-96 h under 25° C. and constant fluorescent illumination and humidity. The tubes were periodically refilled with specific solutions throughout the experiment. Upon termination, the fresh leaves were rinsed with a small amount of water to remove all traces of externally fed precursor, dried gently, frozen, and ground to a fine powder using mortar and pestle. Next, 100 mg of the frozen powdered plant tissue were extracted with 300 μl ethanol, sonicated for 15 min, agitated in an orbital shaker for 30 min, and then centrifuged at 14,000 g for 10 min. The obtained supernatant was filtered through a 0.22 μm syringe filter, and the samples were analyzed in the obtained concentration.

UPLC-qTOF Analysis of Cannabinoids from Helichrysum Tissues

Fresh samples of six different tissues: young leaves, old leaves, florets and receptacle of flowers, stem, and root were collected from a plant at the flowering stage. Florets and the receptacle of flowers were detached using a scalpel and extracted separately. All the tissues were flash-frozen in liquid N2, ground in a mortar to a fine powder, and extracted as previously described with 1 ml ethanol.

Samples were analyzed using a high-resolution ultrahigh-performance liquid chromatography-tandem quadrupole time-of-flight (UPLC-qTOF) system comprised of a UPLC (Waters Acquity) with a diode array detector connected either to a XEVO G2-S QTof (Waters) or to Synapt HDMS (Waters). The chromatographic separation of compounds was performed on a 100 mm×2.1 mm i.d. (internal diameter), 1.7 μm UPLC BEH C18 column (Waters Acquity). The mobile phase consisted of 0.1% formic acid in acetonitrile:water (5:95, v/v; phase A) and 0.1% formic acid in acetonitrile (phase B). The flow rate was 0.3 ml min⁻¹, and the column temperature was kept at 35° C. Cannabinoids were analyzed using a 29 min multistep gradient method: initial conditions were 40% B for 1 min, raised to 100% B until 23 min, held at 100% B for 3.8 min, decreased to 40% B until 27 min, and held at 40% B until 29 min for re-equilibration of the system. Intermediates and glycosylated compounds were analyzed using a 40 min multistep gradient method: from 0% to 28% B over 22 min, raised to 100% B until 36 min, held at 100% B for 2 min, decreased to 0% B until 38.5 min, and held at 40% B until 40 min for re-equilibration of the system. Electrospray ionization (ESI) was used in negative ionization with an m/z range of 50-1,000 Da. Masses of the eluted compounds were detected with the following settings: capillary 1 kV, source temperature 140° C., desolvation temperature 450° C., and desolvation gas flow 800 1 h⁻¹. Argon was used as the collision gas. MS/MS experiments were performed in negative ionization mode according to the observed deprotonated masses. The following settings were used: a capillary spray of 1 kV; cone voltage of 30 eV; collision energy ramp of 15-50 eV.

### Compounds Purification for NMR Analysis

A total of 86 g of fresh leaves were flash-frozen in liquid N2 and ground to a fine powder using an electrical grinder, extracted with 600 ml ethanol, sonicated in an ultrasonic bath for 20 min, and agitated in an orbital shaker at 25° C. for 30 min. Next, the supernatant was filtered under pressure, and the ethanol was evaporated using a rotary evaporator at 40° C. and subsequently lyophilized to remove residual water. The final extract was reconstituted in 25 ml acetonitrile and used for either direct purification (following ten times dilution) or pre-fractionation via medium pressure liquid chromatography (MPLC).

MPLC was performed on a Büchi Sepacore System equipped with two C-605 pump modules, a C-620 control unit, a C-660 fraction collector, a C-640 UV photometer (Büchi Labortechnik AG, Switzerland), and a C18 manually packed column. The mobile phase consisted of acetonitrile:water (5:95, v/v; phase A) and acetonitrile (phase B), with the following multistep gradient method: initial conditions were 0% B for 10 min, raised to 99% B until 530 min, and slowly raised to 100% B until 660 min. The flow rate was 15 ml min⁻¹, the injection volume was 15 ml, and the wavelengths used for monitoring the acquisition were: 210, 224, 270, and 350 nm. Fractions of 100 ml were collected throughout the run giving a total of 99 tubes. The fractions were analyzed by UPLC-qTOF to select specific compounds for purification. The desired fractions were evaporated using a rotary evaporator at 40° C., lyophilized to remove residual water, reconstituted in methanol, and filtered through a 0.22 μm syringe filter.

Purification of compounds was performed on an Agilent 1290 Infinity II UPLC system equipped with a quaternary pump, autosampler, diode array detector, a Bruker/Spark Prospekt II LC-SPE system (Spark), and an Impact HD UHR-QqTOF MS (Bruker) connected via a Bruker NMR MS Interface (BNMI-HP) (the general instrument setup was according to Jozwiak et al.).

The mobile phase consisted of 0.1% formic acid in acetonitrile:water (5:95, v/v; phase A) and 0.1% formic acid in acetonitrile (phase B). Method development was performed by acquisition of both MS and UV signals. MS spectra were acquired in negative full scan mode between m/z 50 and 1,700. The chromatographic separation was performed using XBridge (BEH C18, 250×4.6 mm i.d., 5 μm; Waters) or Luna (C18, 250×4.6 mm i.d., 5 μm; Phenomenex) HPLC columns, and the conditions were adjusted and optimized for each compound. The eluent with the compound of interest was mixed with a makeup-flow of 1.8 ml min⁻¹ water and then trapped on solid-phase extraction (SPE) cartridges (10×2 mm Hysphere resin GP cartridges). Each cartridge was loaded four times with the same compound, and approximately 80 cartridges were used for trapping one compound. Before NMR measurements, SPE cartridges were dried with a stream of N2, and the fraction from each cartridge was eluted with a total of 150 μl MeOH into a 96-well plate. Eluents containing the same compound were pooled, dried under a stream of N₂, and stored at −20° C. until NMR analysis.

### NMR Methods

The purified compounds were resuspended in 300 μl of MeOD-D₄ (Sigma Aldrich), dried under a stream of N₂ to remove traces of ¹H from the previous solvent, reconstituted in 70 μl MeOD-D₄ with 0.01% of 3-propionic-2,2,3,3-D₄ acid sodium salt (that was used as an internal chemical shift reference for ¹H and ¹³C spectra) and transferred into 1.7 mm NMR test tubes for structure elucidation. NMR spectra were recorded on a Bruker AVANCE NEO-600 NMR spectrometer equipped with a 5 mm TCI-xyz CryoProbe. All spectra were acquired at 25° C. The structures of the different compounds were determined by one dimensional (1D) NMR spectra, as well as various two-dimensional (2D) NMR spectra: ¹H-¹H Correlation Spectroscopy (COSY), ¹H-¹H Total Correlation Spectroscopy (TOCSY), ¹H-¹H Rotating Frame Nuclear Overhauser Spectroscopy (ROESY), ¹H-¹³C Heteronuclear Single Quantum Coherence (HSQC), and ¹H-¹³C Heteronuclear Multiple Bond Correlation (HMBC) spectra.

One dimensional ¹H NMR spectra were acquired using 16,384 data points and a recycling delay of 2.5 s. 2D COSY, TOCSY, and ROESY spectra were acquired using 16,384-8,192 (t₂)×400-512 (t₁) data points. 2D TOCSY spectra were acquired using isotropic mixing times of 100-300 ms. T-ROESY spectra were recorded using spin-lock pulses of 100-400 ms. 2D HSQC and 2D HMBC spectra were recorded using 4,096 (t₂)×400-512 (t₁) data points. Multiplicity editing HSQC enables differentiating between methyl and methine groups that give rise to positive correlation versus methylene groups that appear as negative peaks. HMBC delay for evolution of long-range couplings was set to observe long-range couplings of J_{H,C}=8 Hz.

¹H and ¹³C chemical shift assignment was based on information derived from all the NMR spectra. Assignment of the protons as axial or equatorial was based on the observed vicinal J couplings; a large value (>10 Hz) indicates axial protons, further supported by correlations observed in ROESY spectra. ¹H-¹³C correlations observed in HMBC spectra are marked by arrows, and ¹H-¹H correlations observed in COSY spectra are shown by dashed lines.

### Absolute Quantification of CBGA

Samples were extracted as previously described. The final volume was diluted ×10,000 times to fit into the linear range of the calibration curve. Injections were performed on a UPLC (Waters) connected to a Triple Quad detector (TQ-S, Waters) in multiple reaction monitoring (MRM) mode. The chromatographic separation was achieved using a similar column and mobile phase as previously described. A short 7 min method was established using the following multistep gradient program: initial conditions were 57% B raised to 85% B until 4 min, raised to 100% B until 4.2 min, held at 100% B until 6 min, decreased to 67% B until 6.2 min, and held at 67% B until 7 min for re-equilibration of the system. A flow rate of 0.6 ml min⁻¹ was used, the column temperature was 40° C., and the injection volume was 1 μl. The instrument was operated in negative mode with a capillary voltage of 1.5 kV, and a cone voltage of 40 V. Absolute quantification of CBGA was performed by external calibration using two different transitions (359.3>191.2, 32 V for quantification; and 359.3>315.4, 21 V for qualification).

### MALDI Imaging

For localization of terpenophenols to individual trichomes, fresh leaves and flowers were embedded with M1 embedding matrix (Thermo Scientific) in Peel-A-Way disposable embedding molds (Peel-A-Way Scientific) and frozen on dry ice. The embedded tissues were transferred to a cryostat (Leica CM3050) and allowed to thermally equilibrate at −17° C. for at least 2 h. The frozen tissues were sliced into 40 μm-thick sections. The sections were thaw mounted onto Superfrost Plus slides (Fisher Scientific), vacuum dried in a desiccator and imaged with a Nikon DS-Ri2 microscope. A TM sprayer (HTX Technologies) was used to coat the plant tissues with 2,5-dihydroxybenzoic acid (DHB; 40 mg ml⁻¹ dissolved in 70% MeOH containing 0.2% trifluoroacetic acid). The nozzle temperature was set at 70° C. and the DHB matrix solution was sprayed for 16 passes over the tissue sections at a linear velocity of 120 cm min⁻¹ with a flow rate of 50 μl min⁻¹. MALDI imaging was performed using a 7 T Solarix FT-ICR (Fourier Transform Ion Cyclotron Resonance) mass spectrometer (Bruker Daltonics). The datasets were collected in positive ion mode using lock mass calibration (DHB matrix peak: [3DHB+H−3H₂O]+, m/z 409.055408) at a frequency of 1 kHz and a laser power of 40%, with 200 laser shots per pixel and 15 or 25 μm pixel size for the sectioned leaves and flowers, respectively. Each mass spectrum was recorded in the range of m/z 150-3,000 in broadband mode with a Time Domain for Acquisition of 1M, providing an estimated resolving power of 115,000 at m/z 400. The acquired spectra were processed using the Flex-Imaging software 4.0 (Bruker Daltonics). The spectra were normalized to root-mean-square intensity and MALDI images were plotted at theoretical m/z±0.005% with pixel interpolation on.

### Trichome Isolation

Young leaves were harvested and soaked in ice-cold, distilled water and then abraded using a BeadBeater machine (Biospec Products, Bartlesville, OK). The polycarbonate chamber was filled with 15 g of plant material, and with half the volume with glass beads (0.5 mm diameter), XAD-4 resin (1 g/g plant material), and ethanol 80% to full volume. Leaves were beaten by 2-4 pulses of operation of 1 min each. This procedure was carried out at 4° C., and after each pulse the chamber was allowed to cool on ice. Following abrasion, the contents of the chamber were first filtered through a kitchen mesh strainer and then through a 100 μm nylon mesh to remove the plant material, glass beads, and XAD-4 resin. The residual plant material and beads were scraped from the mesh and rinsed twice with additional ethanol 80% that was also passed through the 100 μm mesh. The presence of enriched glandular trichome secretory cells was checked by visualization in an inverted optical microscope.

### Genome Sequencing and Assembly of Helichrysum

The genome size of Helichrysum was estimated by flow cytometry. Briefly, nuclei were isolated by chopping young leaf tissue of Helichrysum and tomato (used as known reference) in isolation buffer. The samples were stained with propidium iodide, and at least 10000 nuclei were analyzed in a flow cytometer, and the ratio of G1 peak means between both samples was calculated. High molecular weight DNA was extracted from young frozen leaves and sent for sequencing in the Genome Center of UC Davis. The DNA quality was checked by TapeStation traces and a Qubit fluorimeter (Thermo Fisher). Sequencing was done in a Pacbio Sequel II platform, and a ˜12-kilobase DNA SMRT bell library was prepared according to the manufacturer's protocol. Three different SMRT 8M cells were used, yielding 57.8 Gb of HiFi data (˜44× haploid coverage). In addition to Pacbio HiFi data, 200 M reads of PE 2×150 Illumina Hi-C data were obtained by Phase Genomics. Hifiasm software was used to integrate both Pacbio HiFi and HiC data to produce chromosome-scale and haplotype-resolved assemblies.

Further scaffolding of the primary assembly was performed using the Hi-C data and the SALSA software. Ragtag was used for a final round of ordering using the primary assembly as reference to reach syntenic scaffolds for each haplotype. Visualizations of Hi-C data were performed with Juicer and whole-genome alignments with the pafr package (dwinter.github.io/pafr/). Finally, the assembly was softmasked for repetitive elements using EDTA.

### Rna Sequencing and Genome Annotation of Helichrysum

RNA was extracted from seven different tissues: young leaves, old leaves, florets and receptacles of flowers, stems, roots and trichomes. RNA integrity was checked using a TapeStation instrument. Paired-end Illumina libraries were prepared for five of the tissues and sequenced on Illumina HiSeq 3000 instrument (PE 2×150, ˜40M reads per sample). Random sequencing errors were corrected using Rcorrector and uncorrectable reads were removed. Adaptor and quality trimming were performed using TrimGalore! with the following parameters: -length 36-q 5-stringency 1 -e 0.1 (github.com/FelixKrueger/TrimGalore). Ribosomal RNA was filtered by discarding reads mapping to SILVA_132_LSURef and SILVA 138_SSURef non-redundant databases using bowtie2-very-sensitive-local mode. Fastq quality checks on each of the steps were performed using MultiQC. The remaining reads were pooled and used for genome-guided de novo transcriptome assembly using Trinity. The Iso-Seq data were obtained from four of the tissues and processed using isoseq3 and cDNA Cupcake ToFU pipelines (github.com/Magdoll/cDNA_Cupcake). Fused and unspliced transcripts were removed, and only polyA positive transcripts were kept for a unique set of high-quality isoforms. Iso-Seq and Trinity transcripts were aligned to the assembly using minimap2 and the BAM files were used in the PASA pipeline to generate RNA-based gene model structures. In addition, the novo gene structures were obtained using the software braker2 and the mentioned BAM files as extrinsic training evidence. Finally, ab initio and RNA-based gene models were combined using EvidenceModeler and a final round of PASA pipeline. Gene functional annotation was performed for the predicted mature transcripts using TransDecoder (github.com/TransDecoder/TransDecoder), which considers HMMER hits against PFAM and BLASTP hits against UniProt databases for similarity retention criteria. Further annotation of protein-coding transcripts was performed by BLASTP searches against curated plant protein databases and GO and KEGG terms were obtained with Triannotate.

UMI-based 3′ RNAseq of three replicates of the seven tissues was obtained similarly as described. Adaptor and quality trimming were performed using TrimGalore! in two steps, including PolyA trimming mode. Reads were mapped to the genome using STAR, UMI-deduplicated using umitools, and counts were obtained with featureCounts. Normalization was performed with the varianceStabilizingTransformation algorithm of DESeq2, and the CEMItools package was used for coexpression analysis (dissimilarity threshold of 0.6, p value of 0.1). Genes in modules with expression profiles in concordance with the presence of the metabolites of interest were analyzed. Candidate genes were selected based on functional annotations, and blast hits with known AAT enzymes.

AAT Expression in E. coli BL21 (DE3) Cells and Protein Purification

Selected AAT genes from Helichrysum were individually cloned into the pET28b vector and expressed in E. coli BL21 (DE3) cells. Bacterial cells were grown overnight in LB medium at 37° C., diluted in fresh LB 1:100, and re-incubated at 37° C. When cultures reached A600=0.6, protein expression was induced with 400 μM of isopropyl-1-thio-β-d-galactopyranoside (IPTG) overnight at 15° C. Bacterial cells were lysed by sonication in 50 mM Tris-HCl pH 8, 0.5 mM phenylmethylsulfonyl fluoride (PMSF, Sigma Aldrich) solution in isopropanol, 10% glycerol and protease inhibitor cocktail (Sigma Aldrich), and 1 mg ml-1 lysozyme (Sigma Aldrich). The whole-cell extract was either kept for functional activity or used for protein purification. Purification of proteins was performed on Ni-NTA agarose beads (Adar Biotech). The proteins were eluted with 200 mM imidazole (Fluka) in a buffer containing 50 mM NaH₂PO₄, pH 8, and 0.5 M NaCl. Protein concentration of the eluted fractions was measured with Pierce™ 660 nm protein assay reagent (Thermo Scientific).

### AAT Enzyme Assay

Recombinant AAT assays with the enzyme solutions using different donor and acceptor substrates were performed by mixing 7 μl of the cannabinoid acceptors (OA, CBGA, or heliCBGA, 1 mg ml⁻¹) with 58 μl of a potassium phosphate buffer (100 mM, pH 7.4) and incubating the mixture at 30° C. for 10 min. Next, 5 μl of the acyl-CoA donors (butyryl-CoA, hexanoyl-CoA, iso-valeryl-CoA, or acetyl-CoA, 10 mM) and 30 μl of the enzyme solutions were added. The reactions were incubated at 30° C. for 3 h. To stop the reactions, 100 μl ethanol was added to each tube, vortexed for 10 s, centrifuged at maximum speed for 10 min, and then the supernatant was recovered and used for UPLC-qTOF analysis. The assay with the purified HuAAT5 enzyme was performed by mixing 2 μl of the cannabinoid acceptors (OA, CBGA, heliCBGA, CBDA, Δ⁹-THCA or CBCA) with 2 μl of the acyl-CoA donors (butyryl-CoA, iso-butyryl-CoA, hexanoyl-CoA, iso-valeryl-CoA, or acetyl-CoA, 10 mM), 44 μl of a potassium phosphate buffer (100 mM, pH 7.4), and 2 μl of the purified HuAAT5 enzyme solution. The reactions were incubated at 30° C. for 3 h. To stop the reactions, 50 μl ethanol was added to each tube and the acylated compounds were extracted and analyzed as previously described.

### Example 1

### UPLC-qTOF Profiling of Helichrysum Tissues

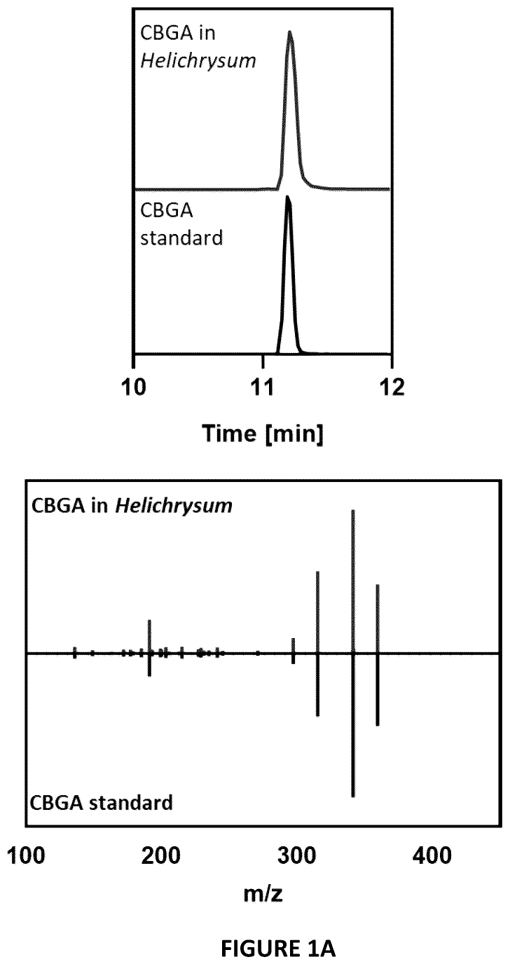

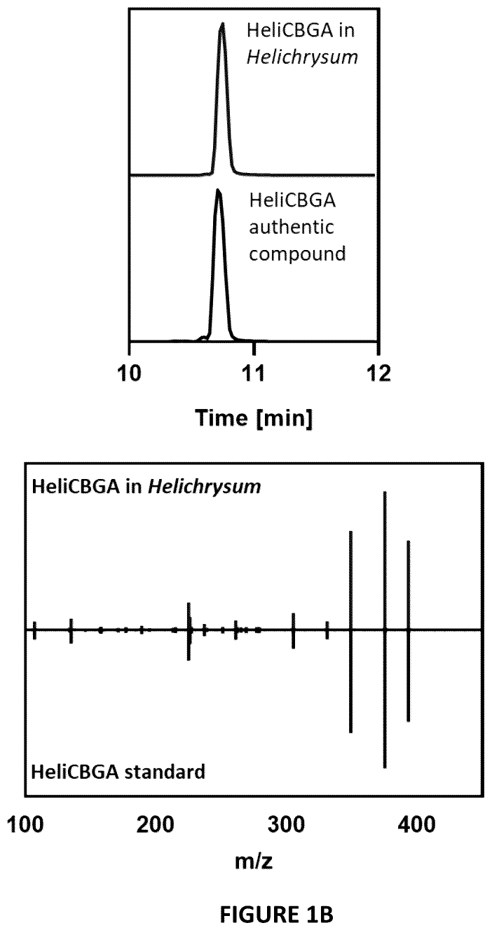

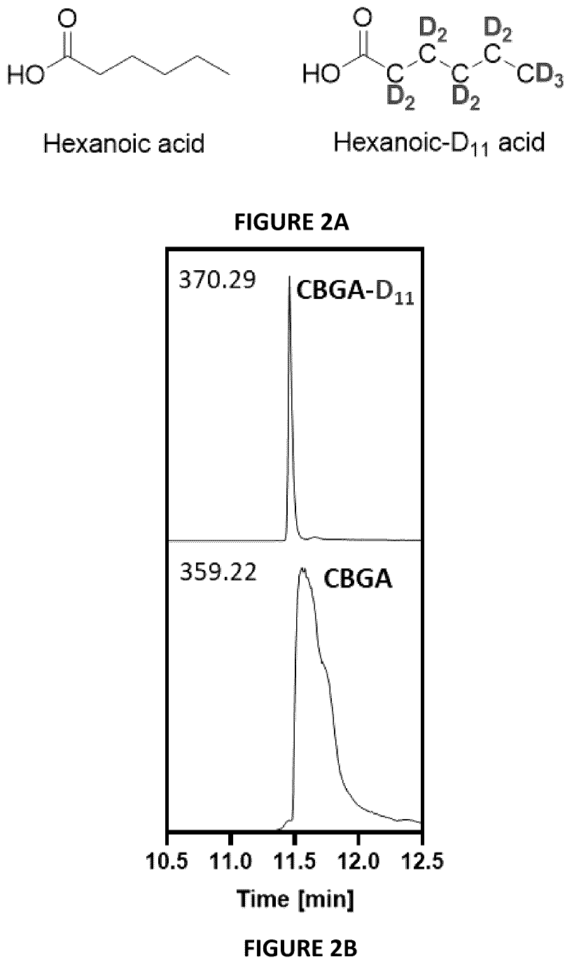

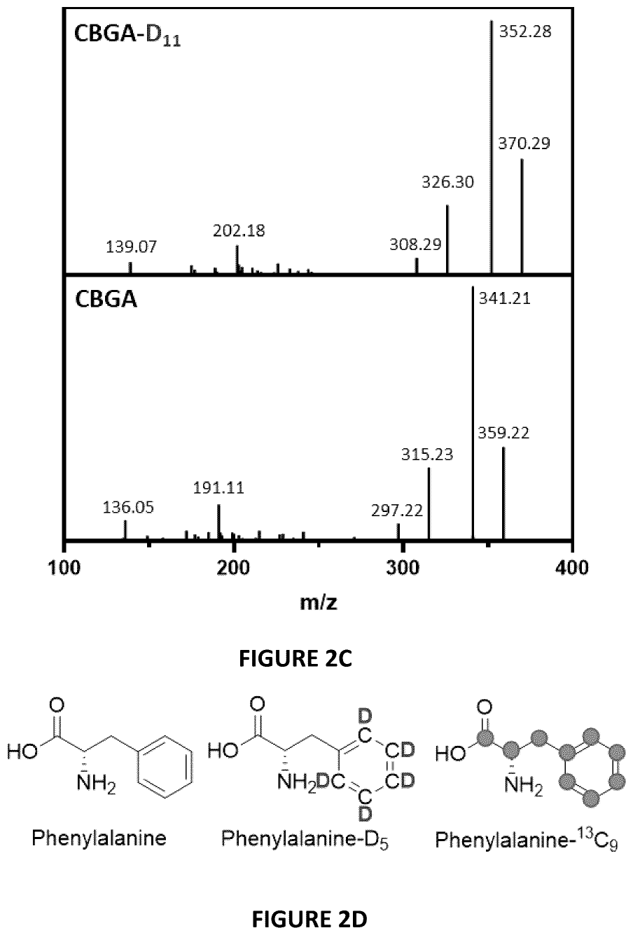

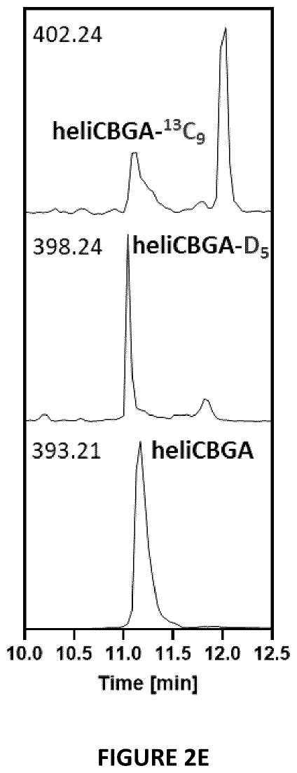

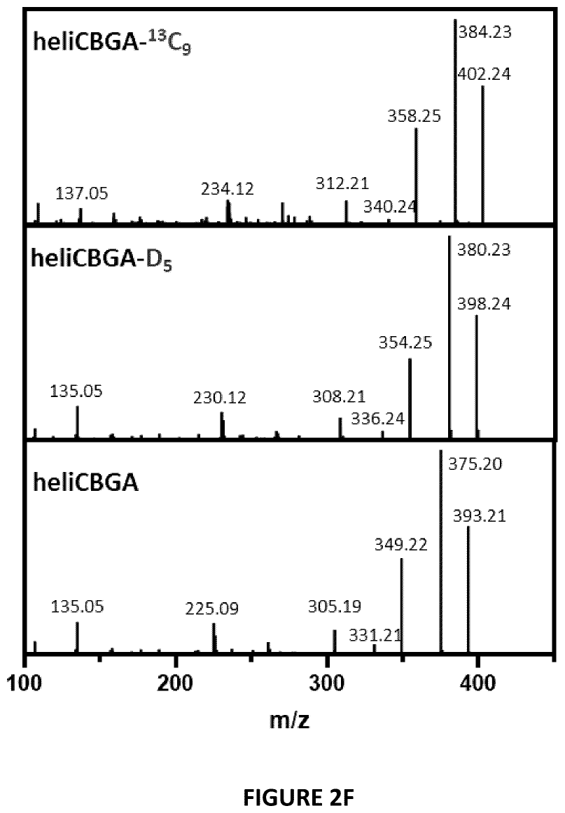

First, the inventors profiled using UPLC-qTOF six tissues (young leaf, old leaf, florets and receptacles of flowers, stems, and roots) of Helichrysum. CBGA and its phenethyl analog heliCBGA were observed in all the tissues besides roots. These compounds were identified by comparison to analytical standards or authentic compounds (FIGS. 1A-1B). Following the elucidated metabolic pathways of cannabinoids and cannabinoid-like compounds in other plants, the inventors demonstrated via feeding of isotopically labeled hexanoic acid (hexanoic-D₁₁ acid) and phenylalanine (phenylalanine-D₅ or phenylalanine-¹³C₉), that these compounds are precursors of CBGA and heliCBGA, respectively (FIG. 2).

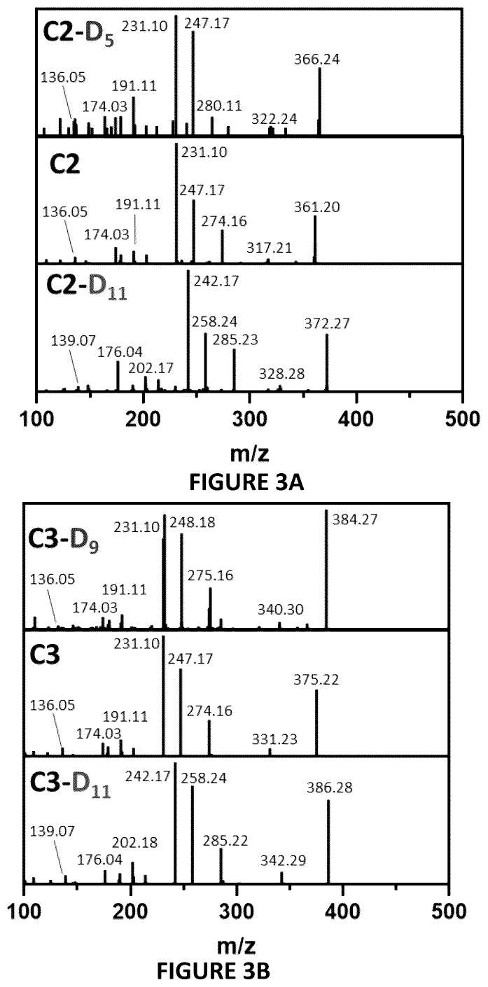

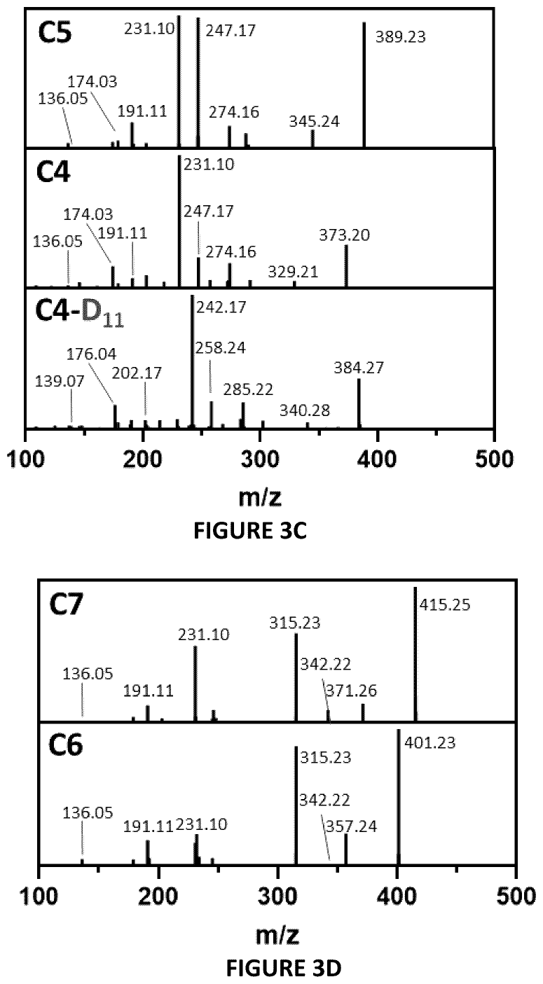

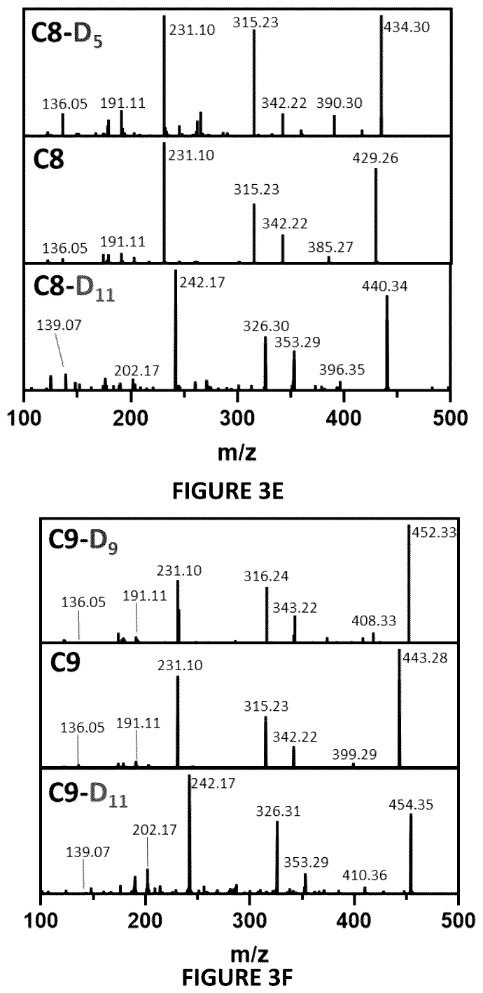

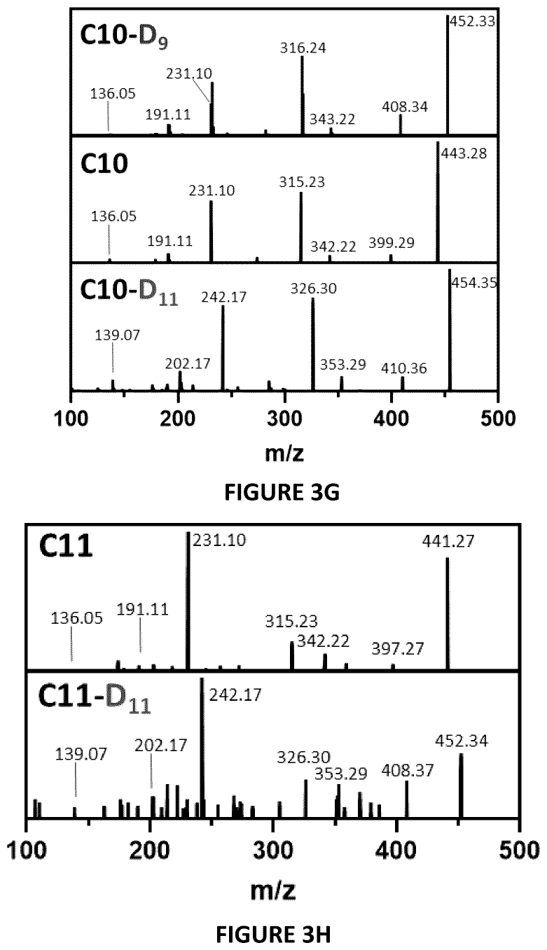

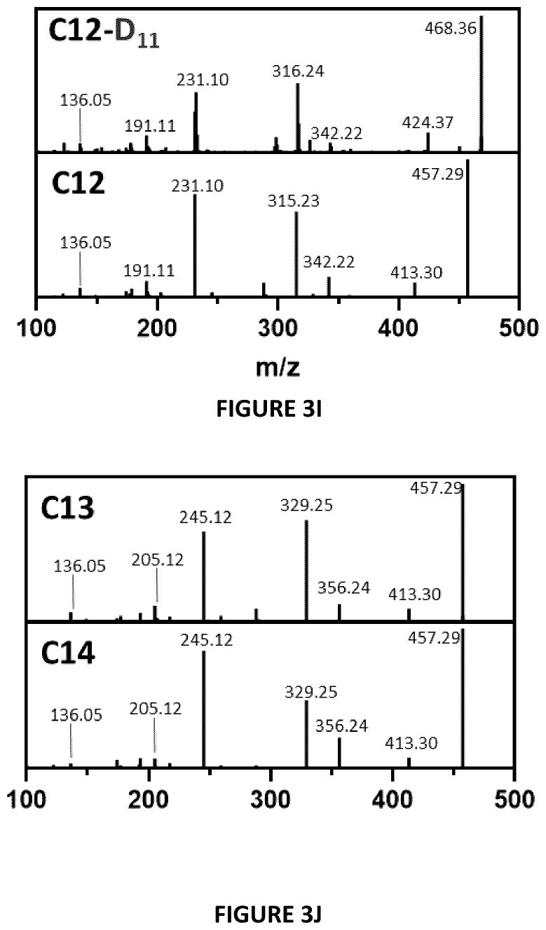

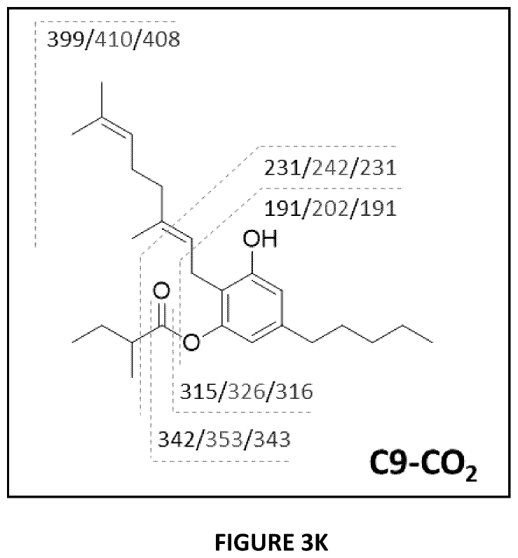

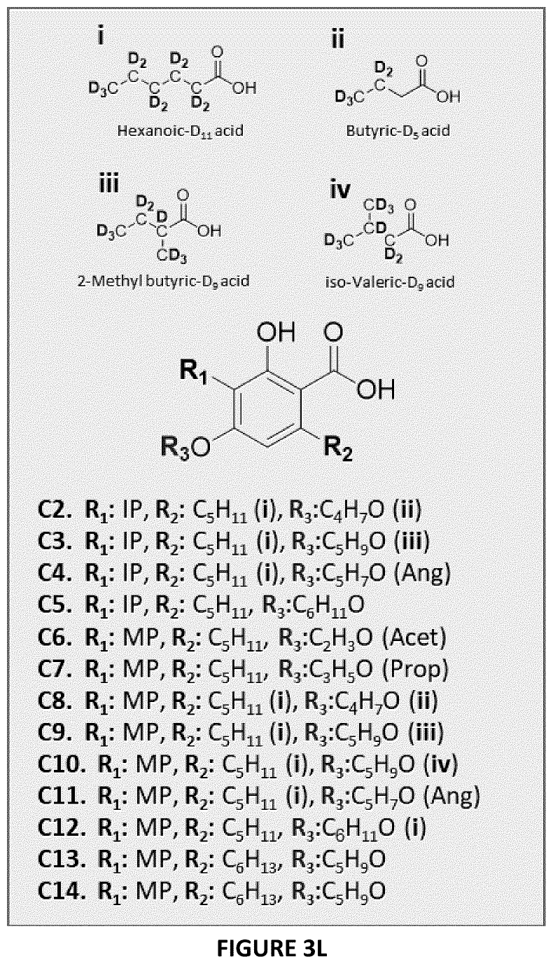

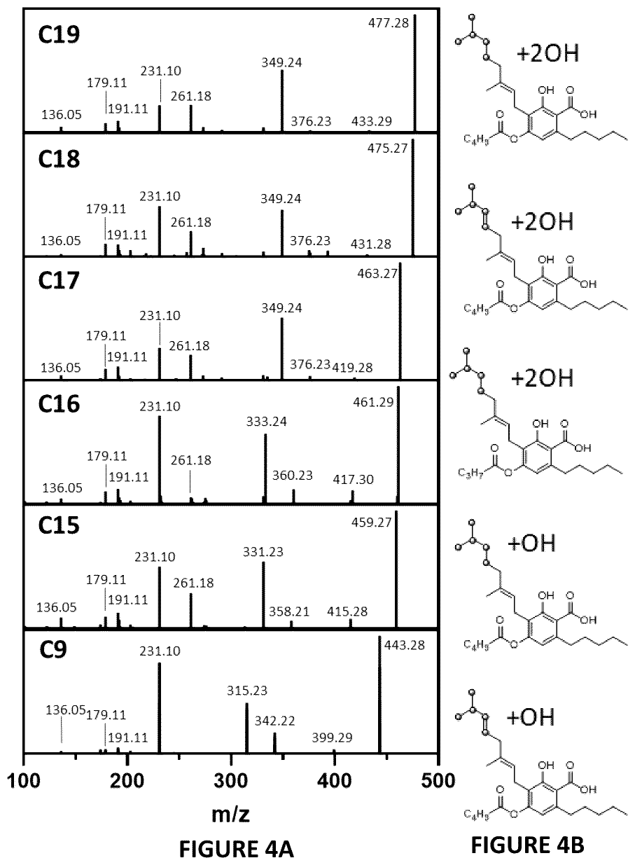

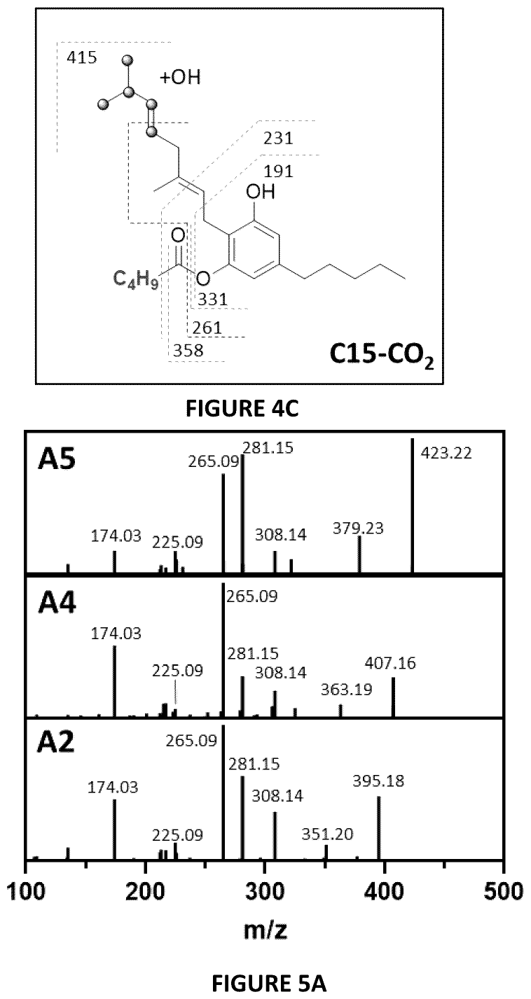

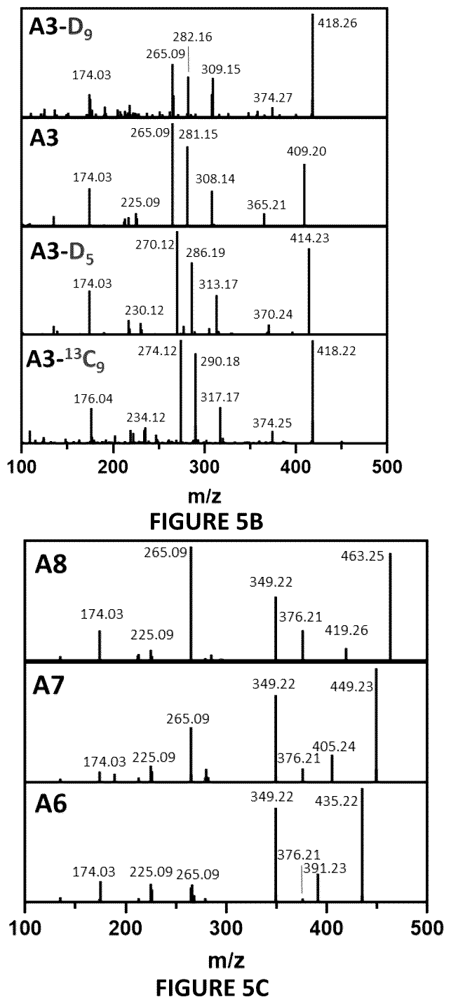

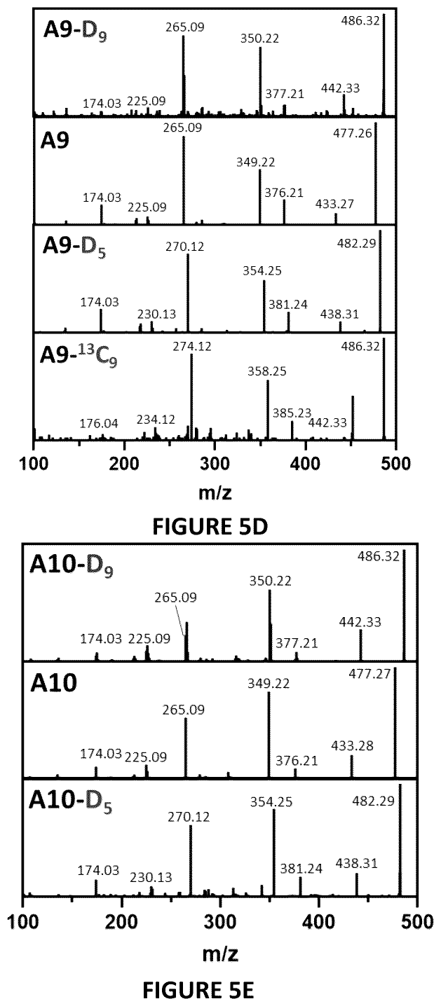

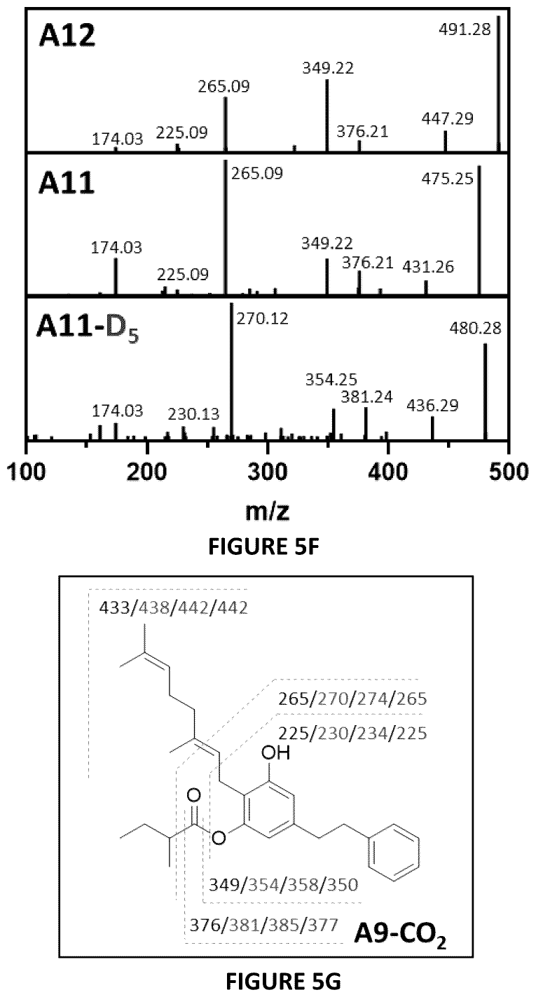

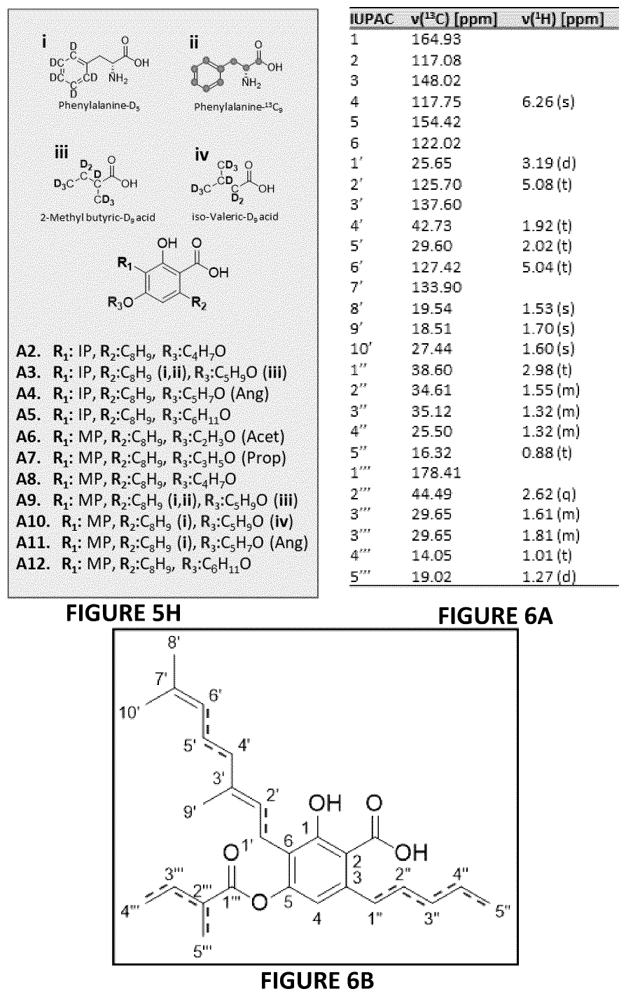

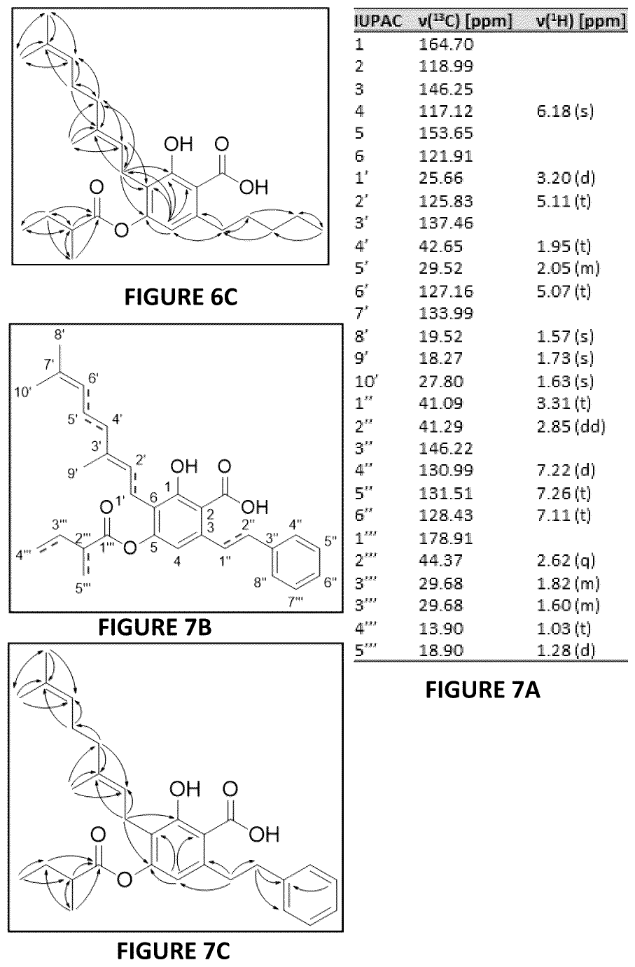

Using stable isotope labeling feeding and MS/MS fragmentation spectra, the inventors further identified a diverse group of O-acylated alkyl (C2-C19, FIGS. 3-4) and aralkyl (A2-A12, FIG. 5) cannabinoids. Previous reports identified in this plant isoprenylated O-acylated compounds from the aralkyl-type but never geranylated or alkyl-type ones. The inventors hypothesized that the acyl group derived from short- or medium-chain FAs and verified this using different isotopically labeled compounds (FIGS. 3L and 5H). Most of the identified alkyl cannabinoids had five-carbon tails (according to labeling with hexanoic-D₁₁ acid, FIG. 3), and both alkyl and aralkyl compounds comprised of iso- or monoprenyls and linear or branched short-chain O-acyl groups, as displayed by the specific labeling. As shown, the position of the FA, either as the alkyl tail or acyl group, can be deduced from the MS/MS fragmentation spectra following feeding with the labeled FAs (FIGS. 3-5). To confirm the identification of this group of compounds, the inventors further purified O-Methylbutyryl-cannabigerolic acid (O-MeButCBGA, C9) and O-Methylbutyryl-helicannabigerolic acid (O-MeButheliCBGA, A9) and analyzed them via NMR (FIGS. 6-7).

### Example 2

### Functional Characterization of AATs

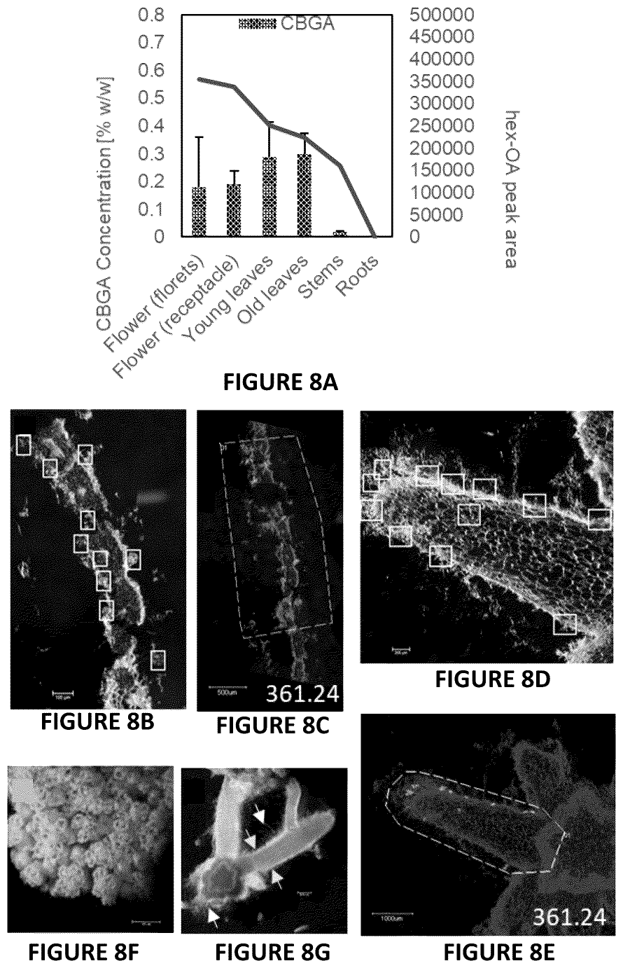

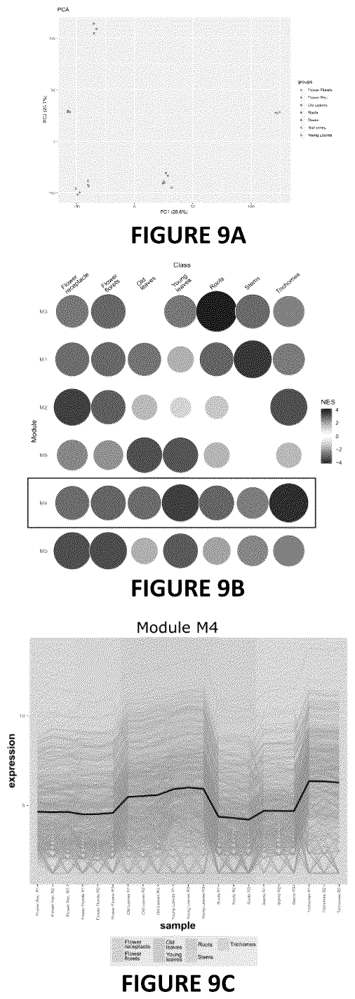

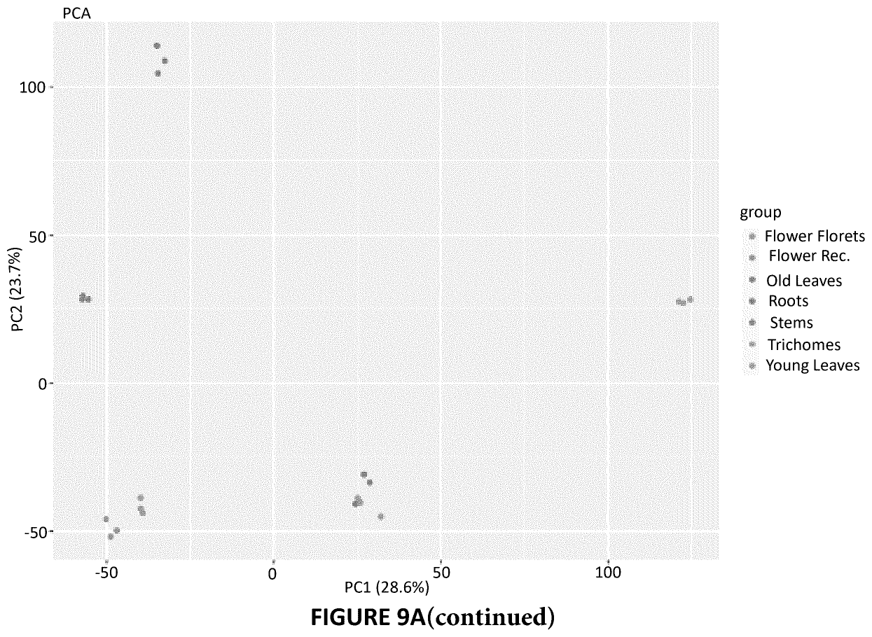

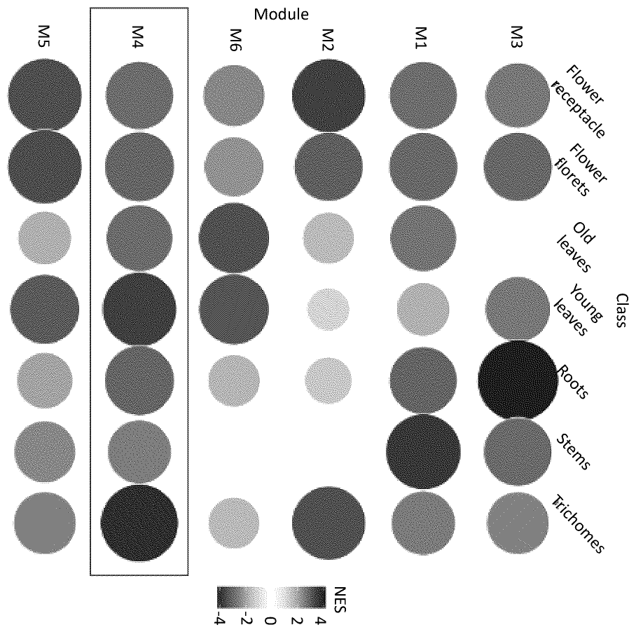

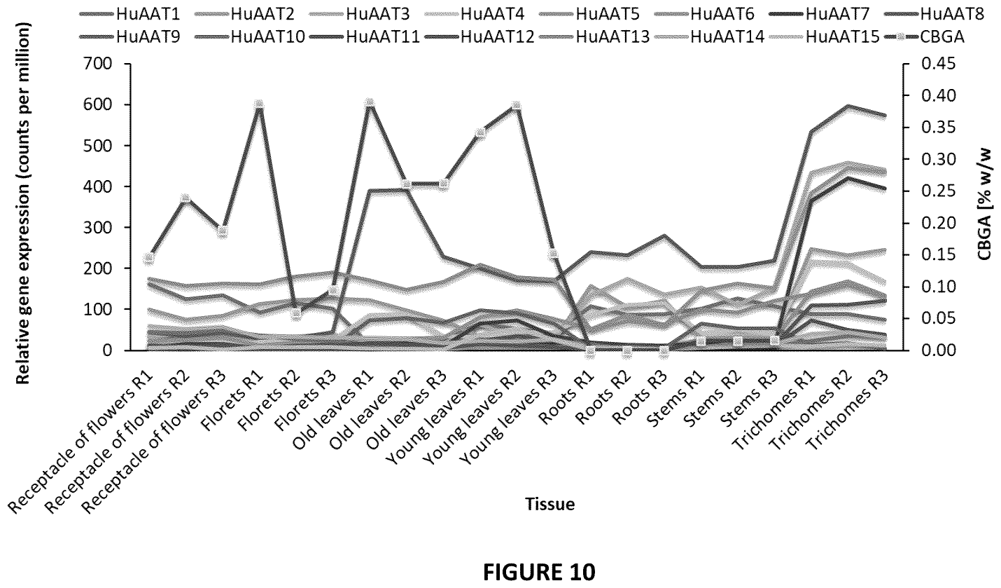

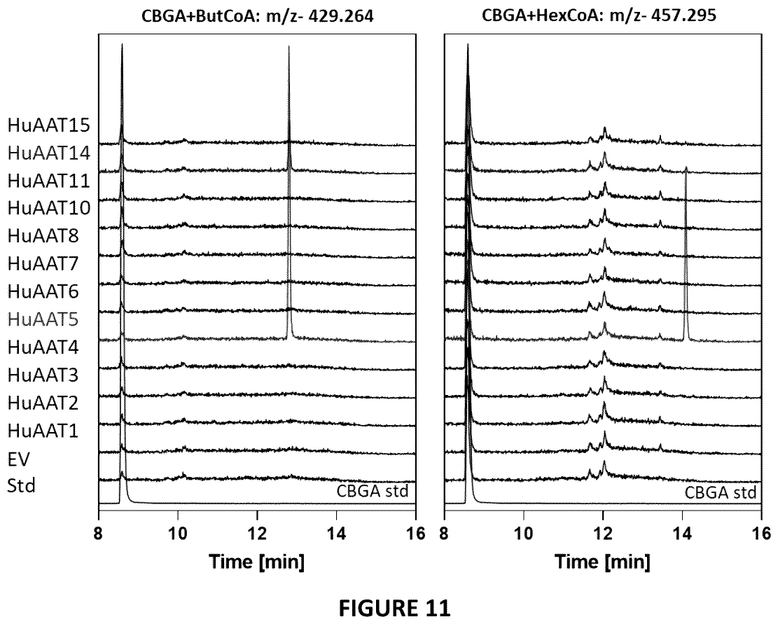

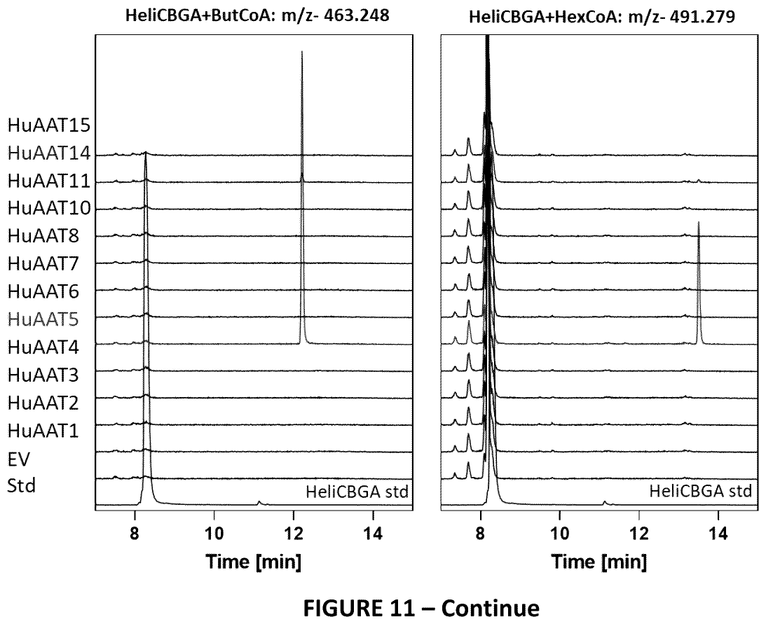

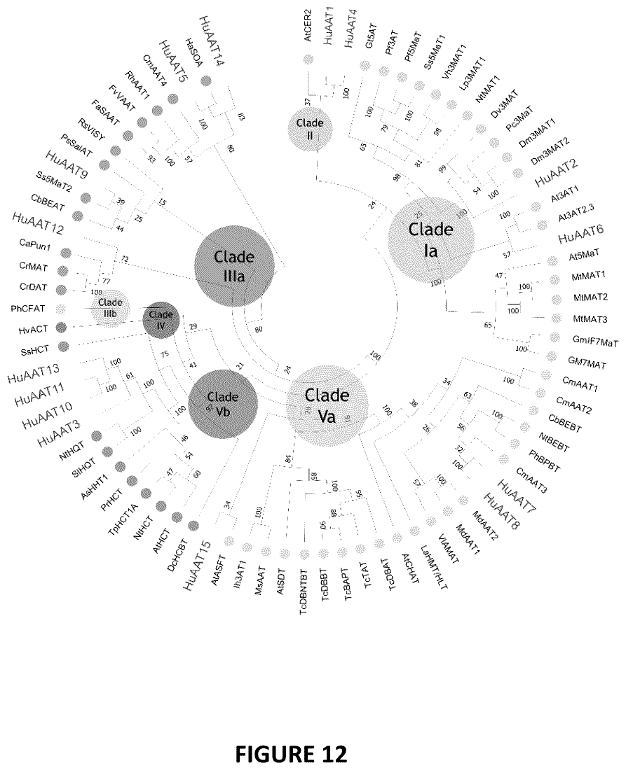

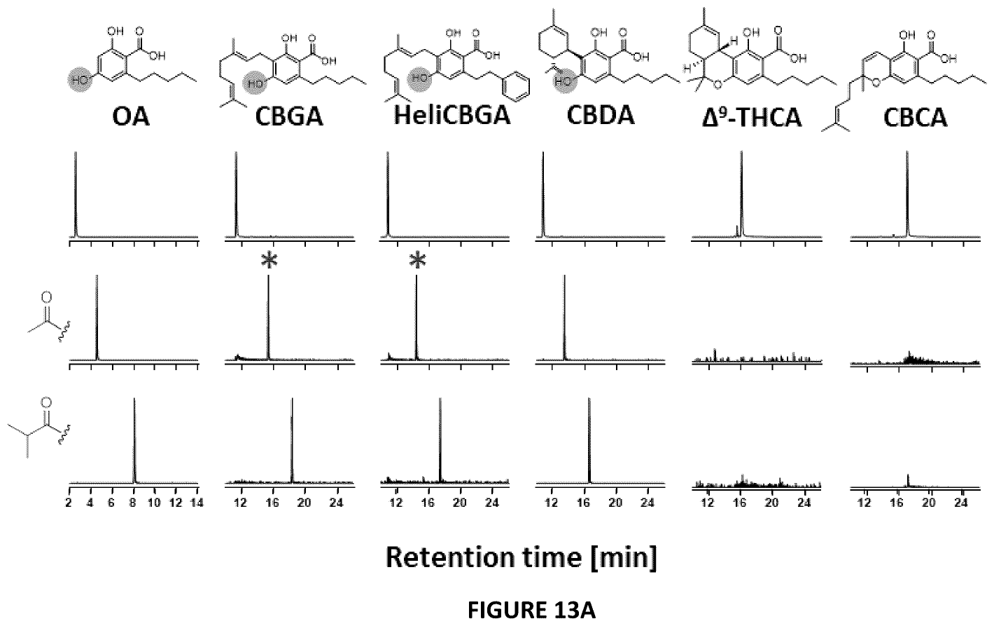

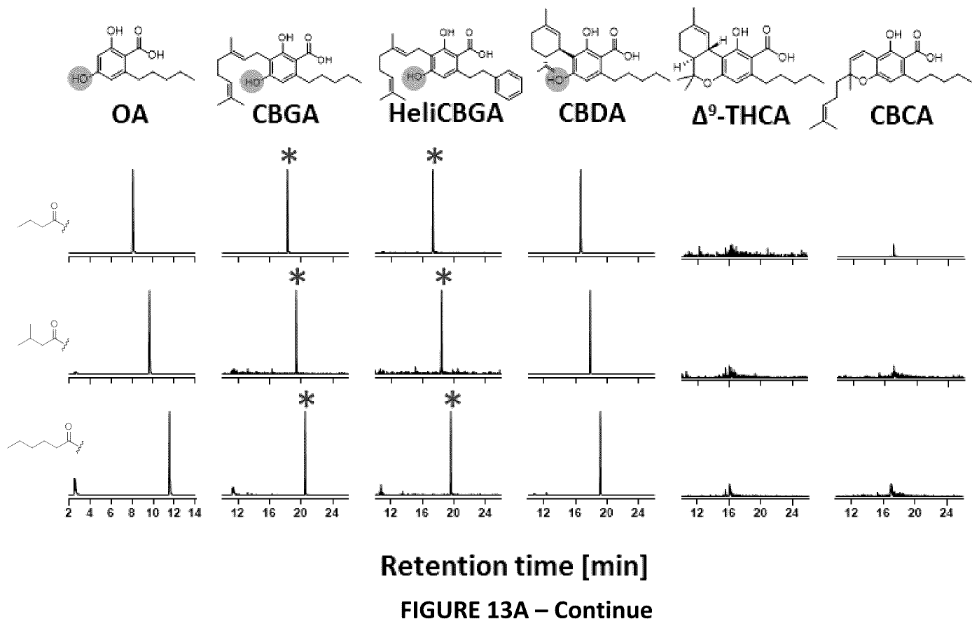

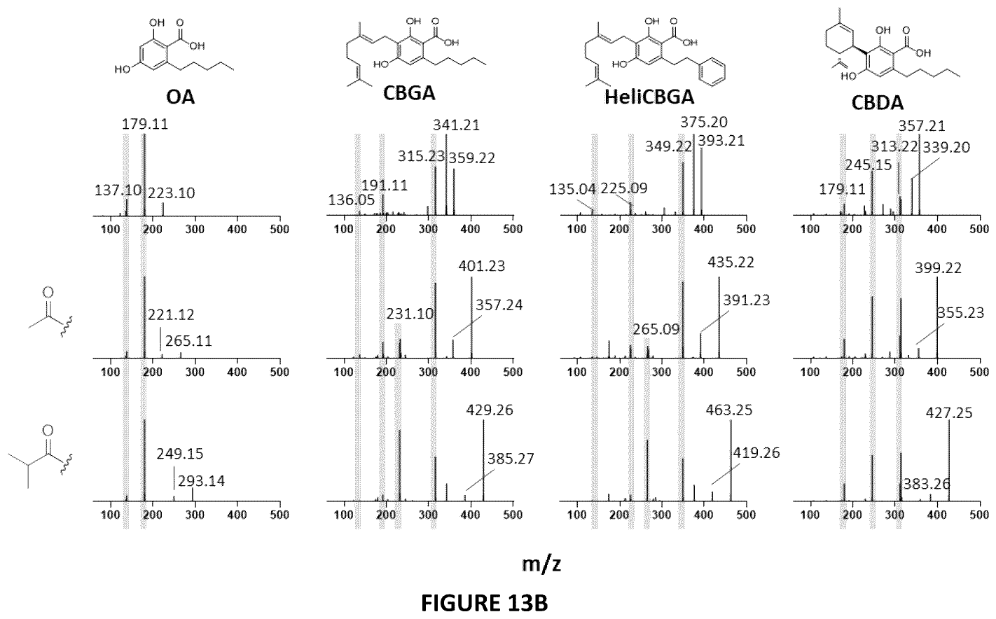

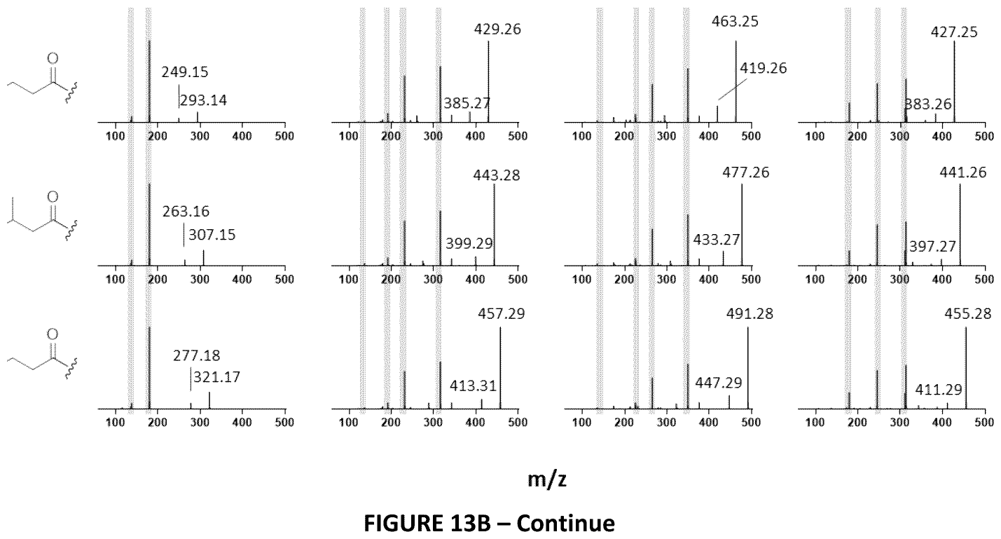

Among the tested tissues in Helichrysum, leaves and flowers showed the highest accumulation of CBGA, while roots contained no CBGA (FIG. 8A). A similar trend was also observed for the O-acylated cannabinoids, as demonstrated for O-MeButCBGA (FIG. 8A). CBGA was further localized to the glandular trichomes of cross-sectioned leaves and flowers via MALDI-MSI (FIGS. 8B-8G). Using this information, RNA-seq transcriptome analysis was performed on these tissues, and candidate genes were selected based on their high expression profile in leaves and flowers compared to roots (FIGS. 9-10). BLAST searches against the Helichrysum genome revealed fifteen (HuAAT1-15) candidate genes that encode putative AATs with high similarity to biochemically characterized BAHD acyltransferases from the literature. Next, the inventors recombinantly expressed twelve of the fifteen AATs from Helichrysum, each separately in E. coli, extracted them, and used the lysate containing the proteins to examine their activity using butyryl-CoA and hexanoyl-CoA as the acyl donors, and CBGA and heliCBGA as the acceptors. From all the tested enzymes, only HuAAT5 (SEQ ID NO: 19) and HuAAT14 (SEQ ID NO: 30) showed activity towards all of these substrates (FIG. 11). Phylogenetic analysis showed that these two enzymes clustered in clade IIIa which represents BAHDs of diverse catalytic functions (FIG. 12). Among the two enzymes, HuAAT5 catalyzed considerably greater amounts of products and was therefore purified to test its activity with an array of acyl donors and acceptors giving rise to natural and unnatural acylated cannabinoids. HuAAT5 accepted all the acyl donors tested and successfully acylated OA, CBGA, heliCBGA and CBDA, giving rise to a single O-acyl-cannabinoid from each pair of substrates (FIG. 12A-12B). On the other hand, the enzyme was inactive on Δ⁹-THCA and CBCA.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. An isolated DNA molecule comprising a nucleic acid sequence having at least 87% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or any combination thereof.

2. The isolated DNA molecule of claim 1, wherein said nucleic acid sequence having at least 87% homology to any one of SEQ ID Nos.: 1-15 is 1,00 to 1,800 nucleotides long, and optionally wherein said nucleic acid sequence encodes a protein being an alcohol acyltransferase (AAT).

3. (canceled)

4. An artificial nucleic acid molecule comprising the isolated DNA molecule of claim 1.

5. A plasmid or an agrobacterium comprising the artificial nucleic acid molecule of claim 4.

6. An isolated protein encoded by isolated DNA molecule of claim 1.

7. The isolated protein of claim 6, comprising or consisting of an amino acid sequence with at least 91% homology to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, and optionally wherein the isolated protein is characterized by being capable of acylating a cannabinoid.

8.-9. (canceled)

10. A transgenic cell comprising the isolated protein of claim 6, optionally wherein said transgenic cell being any one of: a unicellular organism, a cell of a multicellular organism, and a cell in a culture, and optionally wherein said unicellular organism comprises a fungus or a bacterium, and optionally wherein said fungus is a yeast cell.

11.-13. (canceled)

14. An extract derived from the transgenic cell of claim 10, or any fraction thereof, and optionally wherein said extract comprises said isolated protein.

15. (canceled)

16. A transgenic plant, a transgenic plant tissue or a plant part, comprising the isolated protein of claim 6, and optionally wherein said transgenic plant being a Cannabis sativa plant.

17. (canceled)

18. A composition comprising the isolated protein of claim 6 and an acceptable carrier.

19. A method for acylating a cannabinoid comprising:
a. providing a cell comprising an artificial vector comprising a nucleic acid sequence having at least 87% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15; and
b. culturing said cell from step (a) such that a protein encoded by said artificial vector is expressed,
thereby acylating the cannabinoid.

20. The method of claim 19, wherein any one of: (i) said cell is a transgenic cell or a cell transfected with an isolated DNA molecule comprising a nucleic acid sequence having at least 87% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or any combination thereof or a artificial vector comprising thereof; (ii) said protein is characterized by being capable of transferring an acyl group from a donor molecule to said cannabinoid; (iii) said culturing comprises supplementing said cell with an effective amount of a donor molecule comprising an acyl group; (iv) said artificial vector is an expression vector; (v) said cell is a prokaryote cell or a eukaryote cell; (vi) said method further comprises a step (c) comprising extracting said cell, thereby obtaining an extract of the cell, and optionally wherein said method further comprising a step preceding step (c), comprises separating said cultured cell from a medium wherein said cell is cultured; (vii) said method further comprising a step preceding step (a), comprising introducing or transfecting said cell with said artificial vector; and (viii) any combination of (i) to (vii).

21.-27. (canceled)

28. An extract of a cell obtained according to (v) to (vii) of the method of claim 20.

29. A medium or a portion thereof separated from a cultured cell, obtained according to the method of claim 20.

30. A composition comprising the extract of claim 28 and an acceptable carrier.

31. A method for acylating a cannabinoid, the method comprising contacting said cannabinoid or precursor thereof with an effective amount of a protein comprising an amino acid sequence with at least 91% homology to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, thereby acylating the cannabinoid.

32. The method of claim 31, wherein said contacting is in a cell-free system.

33. The method of claim 19, wherein said cannabinoid is CBGA, heliCBGA, CBDA, or any combination thereof.

34. The method claim 19, wherein said cannabinoid is acylated at one or more functional group thereof, being selected from the group consisting of: O, OH, N, NH, NH₂, and any combination thereof.
